(19) United States
(12) Patent Application Publication
(10) Pub. No.: US 2023/0144221 A1
(43) Pub. Date: May 11, 2023

(54) METHODS AND SYSTEMS FOR DETECTING ALTERNATIVE SPLICING IN SEQUENCING DATA

(71) Applicant: Tempus Labs, Inc., Chicago, IL (US)

(72) Inventors: Alessandra Breschi, Mountain View, CA (US); Joshua SK Bell, Chicago, IL (US); Joshua Drews, Downers Grove, IL (US); Benjamin Leibowitz, La Jolla, CA (US); Jackson Michuda, Chicago, IL (US)

(21) Appl. No.: 17/963,969

(22) Filed: Oct. 11, 2022

Related U.S. Application Data
(60) Provisional application No. 63/254,425, filed on Oct. 11, 2021.

(51) Int. Cl. G16B 20/20; G16B 30/10

(52) U.S. Cl. CPC G16B 20/20; G16B 30/10

(57) ABSTRACT

The disclosure provides methods and systems for detecting alternative splicing variants in a patient sample. The methods involve comparison of splice junction data from RNA sequencing with principal splicing isoforms and identifying those sequences, identifying those sequences that do not match the principal splicing isoform. These sequences are categorized into alternative splicing events and documented. Optionally, previously identified target sequences can be utilized in the comparison where the method seeks sequences which do match. Other methods and systems of the present disclosure include those for building a splice profile of alternative splicing variants for a patient sample and those for developing a companion diagnostic test for a treatment method of a disease based on the presence or absence of alternative splicing variants in a patient sample.

### CROSS REFERENCE TO RELATED APPLICATIONS

This application claims priority to U.S. Provisional Patent Application No. 63/254,425, filed Oct. 11, 2021, the content of which is hereby incorporated by reference, in its entirety, for all purposes.

### BACKGROUND

The statements in this section merely provide background information related to the present disclosure and may not constitute prior art.

The ability to perform high-throughput sequencing of RNA (RNA-seq) has transformed understanding of RNA processing and provides a possible source of data for the identification and categorization of the diversity of transcripts that results from alternative splicing. These alternative splicing events are of high interest because of the potential of the association with disease states. It is known that approximately 15-30% of all inherited diseases result in changes in RNA splicing which can be identified as alternative splicing events (see, for example, Lopez-Bigas et al., FEBS Lett. 579:1900-1903 (2005); Wang et al. Nat. Rev. Genet. 8, 749-762 (2007); and Park et al., Am. J. Hum. Genet. 102:11-26 (2018)), and the true impact of alternative splicing on non-inherited disease remains an area of very active research (see, Montes et al., Trends in Gen., 35(1): P68-87 (2019); Taylor et al., Int. J. Mol. Sci. 21: 5161 (2020)). The range of RNA transcripts that are produced by one or more cells (for example, cancer cells) may be referred to as the spliceosome.

This interest is particularly focused in cancer, where drugs that are known to impact the spliceosome are in active development (see, Tang et al. The Scientific World Journal, vol. 2013, Article ID 703568, 8 pages, 2013. https://doi.org/10.1155/2013/703568). In particular, global dysregulation of splicing, as well as mutations in genes regulating splicing, such as SF3B1, have been observed in a variety of tumors (see, Kahles et al., Cancer Cell 34, 211.e6-224.e6 (2018) and Dvinge et al., Nat. Rev. Cancer. 16, 413-430 (2016)). In addition, the results of genome wide association studies (GWAS) focusing on common chronic conditions have identified a number of disease-associated variants that influence splicing, suggesting a role for alternative splicing in mediating many common diseases (see, Li et al. Science 352:600-604 (2016) and Barbeira et al., bioRxiv 814350 (2019)). Furthermore, highly penetrant variants that affect splicing have been classified as pathogenic in a number of monogenic disorders (see, Anna & Monika, J. Appl. Genet. 59, 253-268 (2018)).

A precise detection of alternative splicing events among different biological contexts could provide insights into new molecular mechanisms and help in the development of targeted treatments for patients exhibiting splicing variations. The high-throughput RNA-seq platform is capable of capturing and reporting splicing variants, and several bioinformatics tools have been developed to identify alternative splicing events. There is a need for comprehensive and genome-wide assessments of the splicing events and tools that can provide high-resolution read coverage plots of splicing events with accurate isoform annotation. The primary limitation of tools of the prior art is the low resolution analyzing power and inability to provide well reported detail of the full range of alternative splicing events. Also commonly required are samples from two different time periods or biological events with the analysis being dependent on the ability to compare the two samples in order to identify alternative splicing. Since samples differing in time or conditions (e.g. before and after treatment) are not always available from patients, this is a severe limitation to the practical applicability of the presently available bioinformatic tools.

### SUMMARY

As evident from the description above, there remains a need in the art for methods and systems for determining alternative splicing from RNA-seq data. These methods and systems, as well as other uses of the resulting data such as building splice profiles and developing companion diagnostic tests, should be apparent to those skilled in the art from the present teachings.

This summary is provided to introduce a selection of concepts that are further described below in the detailed description. This summary is not intended to identify key or essential features of the claimed subject matter, nor is it intended to be used as an aid in limiting the scope of the claimed subject matter.

Embodiments of the systems and methods disclosed herein involve methods of detecting alternative splicing variants in a patient sample wherein said variants comprise at least one of exon skipping variants, novel exon addition variants, and novel terminal exon variants, even if such alternative splicing variant had not been previously documented in an annotation reference file; that method comprising, for each gene detected from the RNA-seq reads from the patient sample, comparing splice junction data from the patient sample to a principal RNA isoform reference sequence; identifying those RNA-seq reads that describe exon skipping variants, novel exon addition variants, or novel terminal exon variants through said comparison; documenting at least one of the skipped exons, the added exons, or the terminal exons using a splicing graph for each alternative splicing variant including providing a fully annotated description and splice junction coordinates; and providing in a report an identifier for at least one of the documented alternative splicing variants. This method can further comprise, optionally removing novel splice patterns with overlapping splice sites that are potential false positives using a sample number dependent filter. Additionally, this method can comprise the steps of documenting at least one of the identified alternative splicing variants using a splicing graph including providing splice junction coordinates, and optionally a fully documented annotation of said variant. Additional embodiments of the present method further comprises optionally removing novel splice patterns with overlapping splice sites that are potential false positives using a sample number dependent filter.

Methods of the systems and methods disclosed herein also include the building of a splice profile of alternative splicing variants for a patient sample comprising the steps of comparing splice junction files from the patient sample to the principal RNA isoform for each gene within the RNA-seq reads from the patient sample; identifying those RNA-seq reads that describe exon skipping variants, novel exon addition variants, or novel terminal exon variants through said comparison; optionally documenting at least one of the skipped exons, the added exons, or the terminal exons optionally using a splicing graph or some other documentation for each alternative splicing variant including providing splice junction coordinates and, optionally, a fully annotated version of the splice variant; using the splicing graphs or other documentary information about the variants to produce a patient sample specific isoform dictionary; providing the quantity of reads supporting each entry in the isoform dictionary; and building a report at least associating the isoform dictionary entries with at least one alternative splicing variant to produce the splice profile for the patient sample.

A further embodiment of the methods of the systems and methods disclosed herein are for developing a companion diagnostic test for a treatment method of a disease based on the presence or absence of alternative splicing variants in a patient sample comprising the steps of preparing the splice profiles as described above for a plurality of patients suffering from a disease; associating the treatment response of the patients to a particular treatment method for the disease; determining a further association between positive treatment responses and the presence or absence of particular alternative splice variants in the splice profile for the patient samples; and using the presence or absence of the particular alternative splice variants in a splice profile to identify further patients more likely to benefit from the treatment method than those patients without the presence or absence of the particular alternative splice variants in their splice profile, thus providing a companion diagnostic for the particular treatment method for the disease. This method can be done where the disease is cancer. In certain embodiments of the present method the cancer is selected from group consisting breast cancer, squamous cell cancer, lung cancer, adenocarcinoma of the lung, and squamous carcinoma of the lung, head and neck cancer, cancer of the peritoneum, hepatocellular cancer, gastric cancer, stomach cancer, pancreatic cancer, ovarian cancer, cervical cancer, liver cancer, bladder cancer, hepatoma, colon cancer, colorectal cancer, endometrial or uterine carcinoma, salivary gland carcinoma, kidney or renal cancer, liver cancer, prostate cancer, vulval cancer, thyroid cancer, and hepatic carcinoma, as well as B-cell lymphoma, chronic lymphocytic leukemia (CLL), acute lymphoblastic leukemia (ALL), hairy cell leukemia, chronic myeloblastic leukemia, and post-transplant lymphoproliferative disorder (PTLD). In other embodiments of the present method the cancer is selected from the subgroups of small-cell lung cancer, non-small cell lung cancer (NSCLC), adenocarcinoma of the lung, and squamous carcinoma of the lung, squamous NSCLC, low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, Waldenstrom's Macroglobulinemia, breast cancer subtype Luminal A (hormone receptor (HR)+/human epidermal growth factor receptor (HER2)−); breast cancer subtype Luminal B (HR+/HER2+); breast cancer subtype Triple-negative or (HR−/HER2−); breast cancer subtype HER2 positive; and prostate cancer subtypes involving changes in the ERG, ETV1/4, and FLI1 genes and prostate cancer subtypes defined by mutations in FOXA1, SPOP, and IDH1 genes.

In some embodiments of the methods described herein, the treatment is selected from the group consisting of spliceostatin A, pladienolide-B, GEX1A, E1707, Amiloride, H3B-8800, splice-switching antisense oligonucleotides (SSO), anti-sense oligonucleotides (ASO), short hairpin RNA interference/small interference RNA, clustered regularly interspaced short palindromic repeats (CRISPR)-associated (Cas) systems, CRISPR-Cas13a enzyme, and single-base editors (BEs), cytosine-BEs (CBEs) and adenosine-BEs (ABEs). In some embodiments of the methods described herein, the treatment is selected from inhibitors of the EGFR (Epidermal Growth Factor Receptor), MET (Mesenchymal Epithelial Transition Factor), and AR (Androgen Receptor) genes. Additional embodiments involved methods where the EGFR inhibitor is a tyrosine kinase inhibitor selected from the group consisting of osimertinib, rociletinib, olmutinib, nazartinib, naquotinib, mavelertinib (PF-0647775), and avitinib or an anti-EGFR antibody selected from the group consisting of cetuximab, panitumumab, nimotuzumab, and necitumumab. In some embodiments of the methods described herein, the treatment is a MET inhibitor is selected from the group consisting of crizotinib, tivantinib, savolitinib, tepotinib, cabozantinib, and foretinib or an anti-MET antibody selected from ficlatuzumab and rilotumumab. In some embodiments of the methods described herein, the treatment is an androgen receptor antagonist selected from the group consisting of flutamide, bicalutamide, and nilutamide. The method can also be done where the disease is a thalassemia, familial dysautonomia, spinal muscular atrophy, amyotrophic lateral sclerosis, or Parkinson's disease.

An additional embodiment of the present methods are those methods for detecting, describing, and quantifying RNA molecule variants spliced in a manner alternative to the primary isoform of said RNA molecule from a patient sample, even if such alternative splicing variant had not been previously documented in an annotation reference file, comprising the steps of receiving RNA sequencing data from the patient sample, the sequencing data comprising at least splice junction data to form one or more splice junction files; receiving from an annotation reference file the principal RNA isoform for genes expressed in the patient sample; comparing the splice junction files to the principal isoform files to identify those splice junction patterns that differ from the principal isoform, to detect alternative splice patterns and, optionally, comparing splice junction patterns that match an identified target event splice junction files, to detect target splicing events; categorizing the detected alternative splice patterns into exon skipping events, novel exon events, and terminal exons using comparison to splice junction pairs of the principal isoform file; determining the sequence of the missing exons, if any, from the associated primary isoform file; determining the sequence of the added novel exons from the associated RNA sequencing data, if any; identifying any splice junction data missing a C-terminal member as indication of a terminal exon; building all alternative splicing events into alternative transcripts and all target events into target transcripts and collecting the alternative and the target transcripts into an isoform dictionary; optionally building splicing graphs of all isoform dictionary members including description of any missing exons, any added novel exons, any terminal exons and any target events; and using the splicing graphs or some other documentary evidence of the alternative splicing variant to obtain quantification of all identified alternative splicing and target events within the patient sample sequencing data; calculating the percentage of alternative splicing events as compared to the percentage of principal isoform splicing events, including a calculation of percent spliced in for one or more selected genes in the patient sample; and providing a report table comprising one or more of gene names, alternative splicing coordinates, alternative splicing event descriptions, domain overlaps, number of splicing events, and a Sashimi plot. In some embodiments, the target events are in genes selected from the group consisting of EGFR, MET, and AR.

A still further additional embodiment of the systems and methods disclosed herein are systems for detecting alternative splicing variants in a patient sample wherein said variants comprise at least one of exon skipping variants, novel exon addition variants, and novel terminal exon variants, even if such alternative splicing variant had not been previously documented in an annotation reference file; comprising at least one processor and at least one memory, the system configured to compare splice junction files from the patient sample to the principal RNA isoform for each gene within the RNA-seq reads from the patient sample; identify those RNA-seq reads that describe exon skipping variants, novel exon addition variants, or novel terminal exon variants through said comparison; and document at least one of the skipped exons, the added exons, or the terminal exons using a splicing graph or some other documentation for each alternative splicing variant including providing, optionally a fully annotated description, and splice junction coordinates. Further embodiments include systems further configured to optionally remove novel splice patterns with overlapping splice sites that are potential false positives using a sample number dependent filter. Still additional embodiments include systems further configured to document at least one of the identified alternative splicing variants using a splicing graph including providing splice junction coordinates, and optionally a fully documented annotation of said variant. Some embodiments include systems further configured to update the annotation reference file to reflect the identified alternative splicing variants.

In some embodiments, the systems and methods disclosed herein include systems for building a splice profile of alternative splicing variants for a patient sample, comprising at least one processor and at least one memory, the system configured to compare splice junction files from the patient sample to the principal RNA isoform for each gene within the RNA-seq reads from the patient sample; identify those RNA-seq reads that describe exon skipping variants, novel exon addition variants, or novel terminal exon variants through said comparison; document at least one of the skipped exons, the added exons, or the terminal exons using a splicing graph or some other documentation for each alternative splicing variant including providing, optionally, a fully annotated description and splice junction coordinates; optionally use the splicing graphs or some other documentation of the alternative splice variants to produce a patient sample specific isoform dictionary; provide the quantity of reads supporting each entry in the isoform dictionary; and build a report at least associating the isoform dictionary entries with the quantity of reads supporting each alternative splicing variant to produce the splice profile for the patient sample.

Further embodiments of the systems and methods disclosed herein include systems for developing a companion diagnostic test for a treatment method of a disease based on the presence or absence of alternative splicing variants in a patient sample, comprising at least one processor and at least one memory, the system configured to prepare the splice profiles as described above for a plurality of patients suffering from a disease; associate the treatment response of the patients to a particular treatment method for the disease; determine a further association between positive treatment responses and the presence or absence of particular alternative splice variants in the splice profile for the patient samples; and use the presence or absence of the particular alternative splice variants in the splice profile to identify those patients more likely to benefit from the treatment method, thus providing a companion diagnostic for the particular treatment method for the disease. Certain embodiments of the system is where the disease is cancer. In some embodiments of the systems and methods disclosed herein the cancer is selected from the group consisting of breast cancer, squamous cell cancer, lung cancer, adenocarcinoma of the lung, and squamous carcinoma of the lung, head and neck cancer, cancer of the peritoneum, hepatocellular cancer, gastric cancer, stomach cancer, pancreatic cancer, ovarian cancer, cervical cancer, liver cancer, bladder cancer, hepatoma, colon cancer, colorectal cancer, endometrial or uterine carcinoma, salivary gland carcinoma, kidney or renal cancer, liver cancer, prostate cancer, vulval cancer, thyroid cancer, and hepatic carcinoma, as well as B-cell lymphoma, chronic lymphocytic leukemia (CLL), acute lymphoblastic leukemia (ALL), hairy cell leukemia, chronic myeloblastic leukemia, and post-transplant lymphoproliferative disorder (PTLD). In some embodiments of the systems and methods disclosed herein the cancer is selected from the subgroups of small-cell lung cancer, non-small cell lung cancer (NSCLC), adenocarcinoma of the lung, and squamous carcinoma of the lung, squamous NSCLC, low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, Waldenstrom's Macroglobulinemia, breast cancer subtype Luminal A (hormone receptor (HR)+/human epidermal growth factor receptor (HER2)−); breast cancer subtype Luminal B (HR+/HER2+); breast cancer subtype Triple-negative or (HR−/HER2−); breast cancer subtype HER2 positive; and prostate cancer subtypes involving changes in the ERG, ETV1/4, and FLI1 genes and protate cancer subtypes defined by mutations in FOXA1, SPOP, and IDH1 genes. The present system can be where the treatment method is selected from the group consisting of spliceostatin A, pladienolide-B, GEX1A, E1707, Amiloride, H3B-8800, splice-switching antisense oligonucleotides (SSO), anti-sense oligonucleotides (ASO), short hairpin RNA interference/small interference RNA, clustered regularly interspaced short palindromic repeats (CRISPR)-associated (Cas) systems, CRISPR-Cas13a enzyme, and single-base editors (BEs), cytosine-BEs (CBEs) and adenosine-BEs (ABEs). A further embodiment of the present system is where the treatment method is selected from inhibitors of the EGFR, MET, and AR genes. The system can involve a treatment where the EGFR inhibitor is a tyrosine kinase inhibitor selected from the group consisting of osimertinib, rociletinib, olmutinib, nazartinib, naquotinib, mavelertinib (PF-0647775), and avitinib or an anti-EGFR antibody selected from the group consisting of cetuximab, panitumumab, nimotuzumab, and necitumumab. Additionally, the system can involve a treatment where the MET inhibitor is selected from the group consisting of crizotinib, tivantinib, savolitinib, tepotinib, cabozantinib, and foretinib or an anti-MET antibody selected from ficlatuzumab and rilotumumab. Further, the system can involve a treatment where the AR inhibitor is an androgen receptor antagonist selected from the group consisting of flutamide, bicalutamide, and nilutamide. Other embodiments of the system are where the disease is a thalassemia, familial dysautonomia, spinal muscular atrophy, amyotrophic lateral sclerosis, or Parkinson's disease.

A still further embodiment are systems to detect, describe, and quantify alternative RNA splicing events, even if such alternative splicing has not been previously documented in an annotation reference file, comprising at least one processor and at least one memory, the system configured to receive RNA sequencing data from the patient sample, the sequencing data comprising at least splice junction data to form one or more splice junction files; receive from an annotation reference file the principal RNA isoform for genes expressed in the patient sample; compare the splice junction files to the principal isoform files to identify those splice junction patterns that differ from the principal isoform, to detect alternative splice patterns and, optionally, compare splice junction patterns that match an identified target event splice junction files, to detect target splicing events; categorize the detected alternative splice patterns into exon skipping events, novel exon events, and terminal exons using comparison to splice junction pairs of the principal isoform file; determine the sequence of the missing exons, if any, from the associated primary isoform file; determine the sequence of the added novel exons from the associated RNA sequencing data, if any; identify any splice junction pairs missing a C-terminal member as indication of a terminal exon; build all alternative splicing events into alternative transcripts and all target events into target transcripts and collecting the alternative and the target transcripts into an isoform dictionary; optionally build splicing graphs of all isoform dictionary members including description of any missing exons, any added novel exons, any terminal exons and any target events; use the splicing graphs or other documentation concerning the alternative splice variants to obtain quantification of all identified alternative splicing and target events within the patient sample sequencing data; calculate the percentage of alternative splicing events as compared to the percentage of principal isoform splicing events, including a calculation of percent spliced in for one or more selected genes in the patient sample; and provide a report table comprising one or more of gene names, alternative splicing coordinates, alternative splicing event descriptions, domain overlaps, number of splicing events, and a Sashimi plot. In some embodiments of the systems and methods disclosed herein the target events are in genes selected from the group consisting of EGFR, MET, and AR.

### BRIEF DESCRIPTION OF THE FIGURES

Representative embodiments of Systems And Methods For Detecting Alternative Splicing In Sequencing Data are described with reference to the following figures.

FIG. 1 illustrates an example constitutive RNA splicing event a) and seven exemplary types of alternative splicing events b)-h) (adapted from Jiang et al., Comp. Struct. Biotech. J., 19:183-195 (2021)). As general rules in this Figure, black boxes indicate a sequence that corresponds to a sequence in the constitutive RNA splicing whereas the grey-shaded boxes indicate a sequence in the spliced molecule that differs from the constitutive RNA splicing. Solid lines indicate splicing events present in the constitutive RNA splicing while dotted lines indicate splicing that differs from the constitutive RNA splicing events.

FIGS. 2A and 2B provide an example work flow for the alternative splicing detection method.

FIGS. 3A and 3B provide exemplary alternative splice events and the formula necessary for figuring percent spliced in index (PSI) for each event (adapted from Saraiva-Agostinho and Barbosa-Morais, Nucl. Acids Res. 47(2):e7 (2018)). C1A and AC2 represent the number of sequencing reads supporting junctions between a constitutive (C1 or C2, respectively) and an alternative (A) exon and therefore alternative exon A inclusion, while C1C2 represents the number of sequencing reads supporting the junction between the two constitutive exons. The representative examples here are a) skipped exon, b) skipped exon as a mutually exclusive exon event, c) alternative 5′ splice site and alternative first exon, which share a formula; and d) alternative 3′ splice site and alternative final exon, which also share a formula.

FIG. 4 provides an exemplary splicing graph which can be utilized in the alternative splicing detection method. This splicing graph is of the four transcript variants of the CIB3 gene, specifically variant 1, which comprises exons 1-4, 5, 7-13; variant 2, which comprises exons 1-4, 6-13; variant 3, which comprises exons 1-4, 10-13; and variant 4, which comprises exons 1-2, 8-13 (adapted from Pages et al., https://bioconductor.riken.jp/packages/3.5/bioc/vignettes/SplicingGraphs/inst/doc/SplicingGraphs.pdf).

FIGS. 5A1, 5A2, 5B1, and 5B2 provide exemplary Sashimi plots which can be provided in the report of the alternative splicing detection method. In particular, FIG. 5A is a Sashimi plot for the EGFR gene, as comprised within a report provided by an embodiment of the present method. FIG. 5B is a Sashimi plot for the MET gene, as comprised within a report provided by an embodiment of the present method.

FIGS. 6A, 6B, and 6C collectively show an example block diagram illustrating a computing device and related data structures used by the computing device in accordance with some implementations of the present disclosure.

FIGS. 7A, 7B, 7C, 7D, 7E, 7F, 7G, 7H, 7I, 7J, 7K and 7L collectively illustrate an example method in accordance with an embodiment of the present disclosure, in which optional steps are indicated by broken lines.

### DETAILED DESCRIPTION

In the summary and this detailed description, each numerical value should be read once as modified by the term “about” (unless already expressly so modified), and then read again as not so modified unless otherwise indicated in context. Also, in the summary and this detailed description, it should be understood that a physical range listed or described as being useful, suitable, or the like, is intended that any and every value within the range, including the end points, is to be considered as having been stated. For example, “a range of from 1 to 10” is to be read as indicating each and every possible number along the continuum between about 1 and about 10. Thus, even if specific data points within the range, or even no data points within the range, are explicitly identified or refer to only a few specific data points, it is to be understood that inventors appreciate and understand that any and all data points within the range are to be considered to have been specified, and that inventors possessed knowledge of the entire range and all points within the range.

Prior to setting forth the systems and methods disclosed herein in detail, it may be helpful to the understanding of one of ordinary skill to define the following terms:

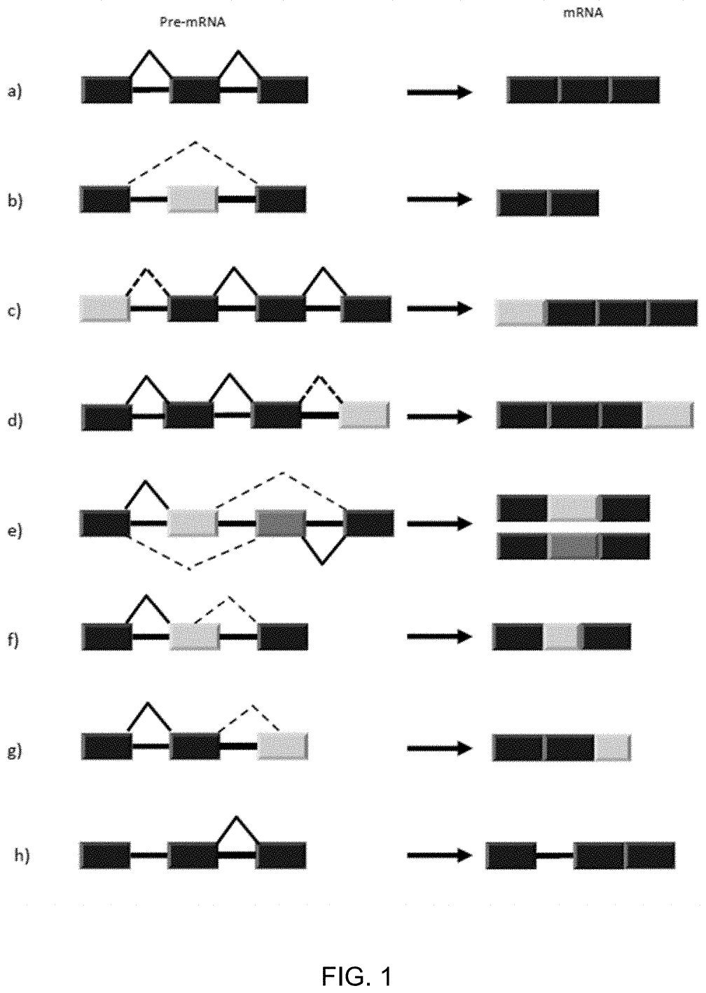

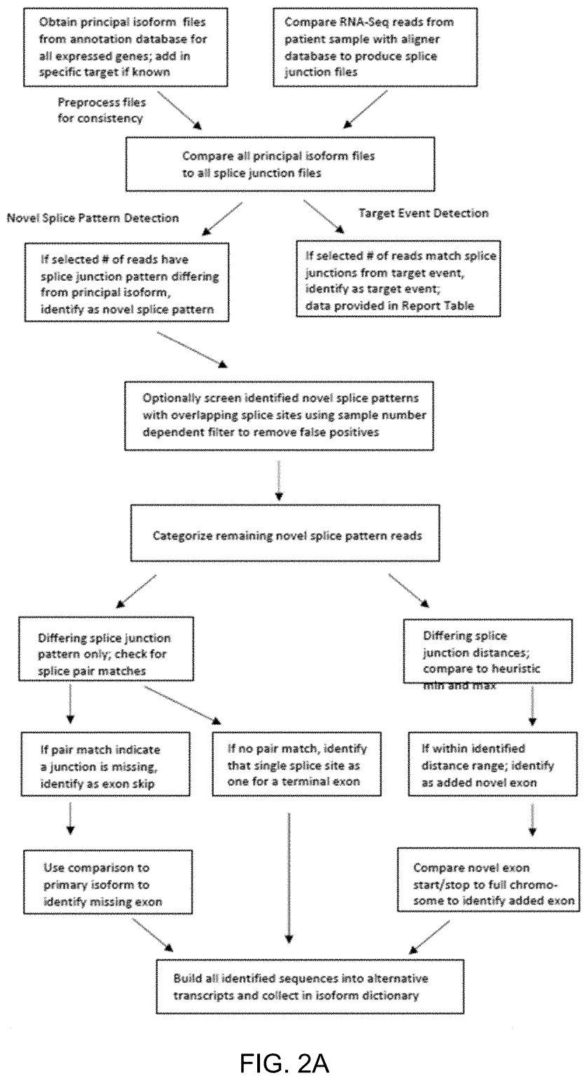

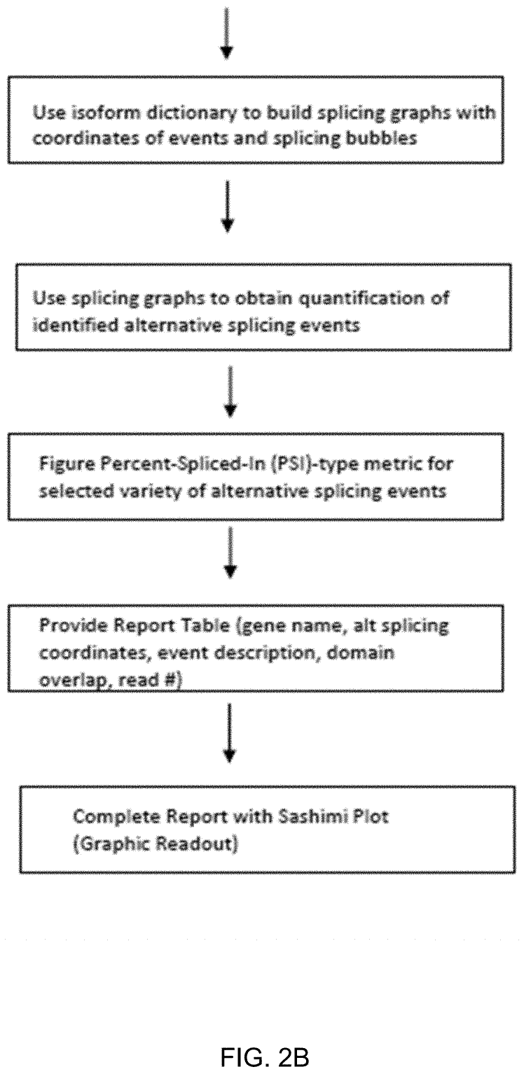

The terms “alternative RNA splicing” or “alternative splicing” are used to denote at least any one of the six major subtypes of alternative splicing events which are illustrated in FIG. 1, b)-h). Specifically, the following are illustrated: b) exon skipping results in complete skipping of one or more exons; c) and d) are novel exon addition variants where c) is the additional of a novel exon on the 5′ end of the RNA and d) is the addition of a novel exon on the 3′ end of the RNA; e) mutually exclusive exons where two or more splicing events are no longer independent, they are executed or disabled in a coordinated manner; f) alternative 5′ splice sites (alternative donors): the usage of an alternative 5′ donor site, which changes the 3′ boundary of the upstream exon; g) alternative 3′ splice sites (alternative acceptors): usage of an alternative 3′ splice junction site causing the change of the 5′ boundary of the downstream exon; and h) novel intron events, also variously known as exon, intron, or intron-exon retention depending on details of the alternative splicing, where one or more introns remain unspliced in the mRNA. However, it should be emphasized that these alternative splicing events are merely illustrative, and any splicing that differs from the constitutive RNA splicing events for a gene, that is, the common splicing isoform set, fall within the scope of this term. Finally, although the Figure illustrates mRNA, any type of RNA that undergoes post-transcriptional processing can have alternative splicing.

The terms “constitutive RNA splicing” or “constitutive splicing” are used to denote the preferred or most commonly seen process of intron removal and exon ligation of the majority of the exons in the order in which they appear in a gene. Constitutive splicing is the process where RNA, for example but not limited to mRNA, is spliced identically producing the same set of common isoforms. The members of this set can be contrasted to the set of various splicing events produced by alternative splicing.

The phrase “novel exon skipping variants” in its most common form describes alternative splicing variants where exons that are generally present in the constitutive RNA splicing events are no longer present in the alternatively spliced variant. It should be understood that this phrase can also describe a variety of alternative splicing variants beyond just the skipping of a single exon as compared to the constitutive RNA splicing sequence, such as alteration of the order of the exons where no sequence is lost, but the sequence order has been rearranged. This phrase can also encompass a subset of the splicing variants known as “mutually exclusive” exon variants as described by Saraiva-Agostinho and Barbosa-Morais, Nucl. Acids Res. 47(2):e7 (2018), particularly when none of the mutually exclusive exons of the variant is present in the constitutive RNA splicing sequence. Thus, these events are exemplarily illustrated in FIG. 1 b)-c).

The phrase “novel exon addition variants” describes alternative splicing variants where exons are either newly added to the RNA sequence as compared to the constitutive RNA splicing sequence or one or more exon sequences have been altered, for example but not limited to, lengthening or shortening the exon sequence as compared to a previously annotated exon. This phrase can also encompass a subset of the splicing variants known as “mutually exclusive” exon variants as described by Saraiva-Agostinho and Barbosa-Morais, Nucl. Acids Res. 47(2):e7 (2018), particularly when at least one, but not all, of the mutually exclusive exons of the variant is present in the constitutive RNA splicing sequence. Thus, these events are exemplarily illustrated in FIG. 1c)-g).

The phrase “novel exon termination variants” describes alternative splicing events where the final exon of a RNA sequence is different than the final exon of the constitutive RNA splicing sequence. This can occur in multiple ways, e.g. through a shortening of the sequence such that an exon that had previously been internal to the encoding is now terminal, or through the addition of exon at the end of the coding sequence that was not present previously. Thus, these events are exemplarily illustrated in FIG. 1 d) and g).

As would be evident to one of ordinary skill, the variant descriptions of the present specification are not mutually exclusive and one alternative splicing event can be described using more than one of these phrases, which are provided to better define the systems and methods disclosed herein, but are not intended to be limiting.

The phrase “principal RNA isoform reference sequence” is a member of the constitutive RNA splicing sequence set that can be selected to be used as the reference sequence in a comparing step of the present methods. The identity of the principal RNA isoform reference sequence for each gene expressed in a patient sample is obtained from the annotation splicing database utilized.

The term “report” denotes a form of clinical or research decision-making support, including clinically or research relevant splice variant information that can be used by a clinician or researcher. Information can include, but is not limited to, alternative splice variants that can be targeted by a therapy or drug; variants that are biomarkers for successful response to a therapy or drug; variants known to affect disease course or prognosis; or variants that can help with diagnosis. Further, the report can merely consist of an overall picture of splicing in the patient or specimen, for example, merely addressing whether there are a greater number or a greater percentage of alternative splice variants compared to a different specimen, for example, a typical specimen.

The phrase “splicing event identifier” refers to a unique label that is provided for at least one novel exon skipping variant, novel exon addition variant, or novel exon termination variant within a report generated by the systems and methods disclosed herein. The identifier is used consistently in reports for multiple patient samples where the same variant is found.

The terms “cancer” refers to or describes the physiological condition in mammals that is typically characterized by unregulated cell growth. Included in this definition are benign and malignant cancers as well as dormant tumors or micrometastases.

As used herein, the term “measure of central tendency” refers to a central or representative value for a distribution of values. Non-limiting examples of measures of central tendency include an arithmetic mean, weighted mean, midrange, midhinge, trimean, geometric mean, geometric median, Winsorized mean, median, and mode of the distribution of values.

As used herein, the term “BAM File” or “Binary file containing Alignment Maps” refers to a file storing sequencing data aligned to a reference sequence (e.g., a reference genome or exome). In some embodiments, a BAM file is a compressed binary version of a SAM (Sequence Alignment Map) file that includes, for each of a plurality of unique sequence reads, an identifier for the sequence read, information about the nucleotide sequence, information about the alignment of the sequence to a reference sequence, and optionally metrics relating to the quality of the sequence read and/or the quality of the sequence alignment. While BAM files generally relate to files having a particular format, for simplicity they are used herein to simply refer to a file, of any format, containing information about a sequence alignment, unless specifically stated otherwise.

As used herein, the term “subject” refers to any living or non-living organism including, but not limited to, a human (e.g., a male human, female human, fetus, pregnant female, child, or the like), a non-human mammal, or a non-human animal. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a male or female of any age (e.g., a man, a woman, or a child). In some embodiments, a subject is a human.

As used herein, the terms “expression level,” “abundance level,” or simply “abundance” refers to an amount of a gene product, (an RNA species, e.g., mRNA or miRNA, or protein molecule) transcribed or translated by a cell, or an average amount of a gene product transcribed or translated across multiple cells. In some embodiments, an expression level can refer to the amount of a particular isoform of an mRNA corresponding to a particular gene that gives rise to multiple mRNA isoforms. The genomic locus can be identified using a gene name, a chromosomal location, or any other genetic mapping metric.

As used herein, the terms “sequencing,” “sequence determination,” and the like refer to any biochemical processes that may be used to determine the order of biological macromolecules such as nucleic acids or proteins. For example, sequencing data can include all or a portion of the nucleotide bases in a nucleic acid molecule such as an mRNA transcript or a genomic locus.

All references cited herein are incorporated by reference in their entirety.

In some embodiments, the systems and methods disclosed herein are based in part upon the discovery of computational methods and systems for identifying and describing alternative splicing from RNA-seq data derived from patient samples. These methods and systems and the data produced therefrom can be further utilized for the production of patient splicing profiles and the development of companion diagnostics for treatment methods utilized to treat disease where the response to the treatment method has been shown to be related to the characteristics of the obtained splicing profile, and the identification of possible drug targets (for example, splice variants that occur at or above a certain rate in a particular patient population) to be used for drug development.

### A. Description of RNA-seg

In some embodiments, the systems and methods disclosed herein utilize data produced by next generation sequencing of RNA (RNA-seq). The original goal of RNA-seq was to identify which genetic loci are expressed in a cell (population) at a given time over the entire expression range without the need to pre-define the sequences of interest as was the case with cDNA microarrays. RNA-seq has proven to be able to identify even lowly expressed transcripts with a very low level of false positives, especially when compared to cDNA microarrays. In addition, RNA-seq can be used not only for the quantification of expression differences between distinct conditions, it also offers the ability to detect and quantify other RNA transcripts present in cells, such as non-protein-coding transcripts, novel transcripts, sites of protein-RNA interactions, and splice isoforms. It is the identification, quantification, categorization, and documentation of this final type of RNA transcript within the RNA-seq data reads that is the focus of the systems and methods disclosed herein.

The present method contemplates starting with some sort of tissue sample of which information about the entire transcriptome is desired without the necessity of identifying target sequences in advance, although such identification can be an optional approach. This is generally done using total RNA sequencing which can accurately measure gene and transcript abundance, and identify known and novel features of the transcriptome. The present method is contemplated to be able to be practiced with total RNA sequencing, it can be equally practiced with a probe captured subset of the total set (see, for example probe panels used for whole exome sequencing (WES, as described in Rabbani et al., J. Hum. Genet., 59:5-15 (2014); Suwinski et al., Front. Genet. 12 Feb. 2019), or another targeted panel of selected genes (e.g. various selected subsets of less than the whole transcriptome) or with the RNA obtained through poly-A capture. More details about this approach are provided below. The sample can be derived directly from a patient either at a tissue sample or some sort of bodily fluid sample, or alternatively, an artificial organoid which is grown from tissue or sample provided from a patient. Samples from archival tissues, where exosomes may be the most rich source of RNA are also contemplated by the systems and methods disclosed herein. When RNA-seq data is desired from a patient sample or an organoid, the first step is the isolation of the RNA from that sample. Methods of RNA isolation are well known in the art and vary depending on the precise tissue or sample type involved. Important considerations include stabilization of the RNA after collection, ensuring complete or substantially complete sample lysis, eliminating or substantially eliminating DNA contamination, and choosing from the variety of RNA isolation kits which is highly dependent on the original RNA source. For examples of RNA isolation techniques, see Conesa A et al., Genome Biol. 17:13 (2016).

While direct sequencing of RNA molecules is possible, most RNA-Seq experiments are carried out on instruments that sequence DNA molecules due to the technical maturity of commercial instruments designed for DNA-based sequencing. Therefore, cDNA library preparation from RNA is a required step for many embodiments of RNA-Seq. Each cDNA in an RNA-Seq library is composed of a cDNA insert of certain size flanked by adapter sequences, as required for amplification and sequencing on a specific platform. The cDNA library preparation method varies depending on the RNA species under investigation, which can differ in size, sequence, structural features and abundance. Major considerations include (1) how to capture RNA molecules of interest; (2) how to convert RNA to double-stranded cDNAs with defined size ranges; and (3) how to place adapter sequences on the cDNA ends for amplification and sequencing.

In some embodiments, sequencing of polyadenylated RNA is used in the systems and methods disclosed herein, to allow focus on alternative spliced reads. In eukaryotic organisms, most protein-coding RNAs (mRNAs) and many long noncoding RNAs (incRNAs) (>200 nt) contain a poly(A) tail. The poly(A) tail provides technical convenience for enrichment of poly(A)+RNAs from total cellular RNA, in which they account for approximately 1-5% of the pool. Poly(A)+RNA selection can be carried out with magnetic or cellulose beads coated with oligo-dT molecules. Alternatively, polyadenylated RNAs can be selected using oligo-dT priming for reverse transcription (RT). While efficiently incorporating both poly(A) selection and RT in one step, oligo-dT priming-based methods can exhibit 3′ bias, resulting in sequencing reads enriched for the 3′ portion of the transcript. In addition, oligo-dT can frequently prime at internal A-rich sequences of transcripts, a phenomenon called internal poly(A) priming, leading to biased RT. Therefore, poly(A) purification is a preferred method to select poly(A)+RNA unless a very low amount of RNA is available. However, it should be noted that non-polyadenylated RNAs such as fragmented mRNAs from formalin-fixed, paraffin-embedded (FFPE) samples could be of interest using the systems and methods disclosed herein and thus specialized methods of isolation should be utilized, such as those described in Pennock et al., BMC Medical Genomics, 12: 195 (2019).

A major issue in sequencing these RNAs is how to eliminate ribosomal RNAs (rRNAs), which are the most abundant RNA species in the cell but of little interest for the systems and methods disclosed herein and their focus on alternative splicing. Several approaches have been developed to deplete them from the RNA pool. One approach to eliminate rRNAs is based on sequence-specific probes that can hybridize to rRNAs. Unwanted rRNAs or their cDNAs are hybridized with biotinylated DNA or locked nucleic acid (LNA) probes, followed by depletion with streptavidin beads. Alternatively, rRNAs are targeted by anti-sense DNA oligos and digested by RNase H, a method also known as probe-directed degradation (PDD). While this approach is less laborious than hybridization, it may require continuous coverage of rRNAs and unique probe sets. A noncontinuous sequence-based method was recently developed which has addressed some of these issues. In this method, all cDNAs, including those of rRNAs and other RNAs, are circularized, and are hybridized to rRNA probes. The hybridized sequences are then digested by duplex-specific nuclease (DSN), making them unusable for amplification. However, this approach requires high input amounts of total RNA, which can be challenging when dealing with clinical samples.

Another approach for rRNA reduction uses specific, not-so-random (NSR) primers which bind to the RNA molecules of interest during RT, thus avoiding rRNAs. This method, commercialized as Ovation RNA-Seq (Tecan, Mannedorf, Switzerland), uses hexamer or heptamer primers whose sequences are absent from rRNAs. Similar to this approach, one study used 44 heptamers to avoid both rRNAs and highly-expressed transcripts. In this way, only 40 primers for RT instead of 700 NSR primers were needed, which works well with partially degraded RNA and low-input samples. In addition to the sequence-based approaches mentioned above, some methods take advantage of certain features of rRNAs for their elimination. The COT-hybridization method is based on heat denaturation, re-annealing and selective degradation by DSN. Double-stranded cDNAs originating from abundant sequences are preferentially degraded because of their more rapid annealing kinetics compared to less abundant ones. Selective degradation has also been achieved by using the enzyme terminator 5′-phosphate-dependent exonuclease (TEX), which recognizes RNA molecules with 5′-monophosphate, including rRNAs and tRNAs.

A common clinical starting point is a patient blood sample, in which case a frequently used technique is globin depletion, which employs probe-based removal or inhibition of hemoglobin-related transcripts. This can greatly increase the relative number of reads that will be generated from non-globin RNA, since globin transcripts comprise between 50-80% of blood RNA (see, Mastrokolias et al., BMC Genomics, 13:28 (2012)).

In summary, as well known by one of ordinary skill, the selection of an approach for enriching RNA transcripts of interest for sequencing depends on the goal of the experiment and many technical factors. Several studies have compared protocols for removal of rRNA by depletion- and priming-based methods. In eukaryotic cells, oligo-dT bead-based purification of poly(A)+RNA is the method of choice for most applications, because of its ease of use and relatively low cost. For low-input samples, however, oligo-dT priming generally offers better results.

After poly(A)+selection or rRNA depletion, RNA samples are typically subject to RNA fragmentation to a certain size range before RT. In certain embodiments, t his is necessary because of the size limitation of most current sequencing platforms. RNAs can be fragmented with alkaline solutions, solutions with divalent cations, such Mg++, Zn++, or enzymes, such as RNase III. Fragmentation with alkaline solutions or divalent cations is typically carried out at an elevated temperature, such as 70° C., to mitigate the effect of RNA structure on fragmentation. Alternatively, intact RNAs can be reverse transcribed, and full-length cDNA can be fragmented. A traditional method to fragment cDNA requires the use of acoustic shearing. Alternatively, full-length double-stranded cDNAs can be fragmented by DNase or a tagmentation method can be used to fragment cDNA and add adapter sequences at the same time. In this method, an active variant of the Tn5 transposase mediates the fragmentation of double-stranded DNA and ligates adapter oligonucleotides at both ends in a quick reaction (˜5 min) (see, Picelli et al., Genome Res. 2014; 24:2033-2040). However, it is notable that Tn5 and other enzyme-based cDNA fragmentation methods may require a precise enzyme:DNA ratio, making method optimization less straightforward than RNA fragmentation. Consequently, fragmenting RNA is currently still the most frequently used approach in RNA-Seq library preparation.

In a standard RNA-Seq library protocol, cDNAs of a desired size are generated from RT of fragmented RNAs with random hexamer primers or from fragmented full-length cDNAs that are ligated to DNA adapters before amplification and sequencing. Due to the detection limit of most sequencers, cDNA libraries may need to be amplified by a polymerase chain reaction (PCR) process before sequencing. While only a small number of amplification cycles (8-12) are used during most embodiments of PCR, variations in cDNA size and composition can result in uneven amplification. Amplification of some cDNAs plateau while others continue to amplify exponentially. To correct for PCR amplification bias, methods that eliminate PCR duplicates from sequencing results may be used. In one method, under the assumption of random RNA fragmentation, final sequencing reads having the same start and stop coordinates are considered as PCR duplicates and are merged. Another method is to use molecular labels, also known as unique molecular identifiers (UMIs), to distinguish PCR products. Molecular labels are typically introduced within the adapter sequence, prior to PCR amplification. In a modified protocol for making cDNAs from single cells, molecular labels are introduced by the Tn5 transposase during fragmentation of double-stranded, amplified cDNA. However, in some applications, such as digital counting of targeted RNAs, molecular labels are added during RT. Molecular labels differ in size (number of bases) and complexity. In principle, they comprise either defined sequences or random nucleotides. Defined sequences, chosen for their even distribution in final libraries, are more technically challenging to make in some embodiments because of sequence selection and manufacturing complexity. By contrast, random sequences, while easy to implement, give high variability among molecular labels. Molecular labeling is particularly valuable in situations where input RNA is scarce and a large number of PCR cycles is required for sequencing, such as single-cell RNA-seq. Although the present methods anticipate the utilization of traditional RNA-seq approaches as described above, it is also anticipated that single-cell RNA-seq and related methods, for example but not limited to those that begin with less input material, could be the source of reads for use in the present method.

A further method that can be utilized is a combination of RNA-Seq with exome enrichment (see, for example Cieslik et al., Genome Res. 25(9):1372-81 (2015)). This method involves utilizing a panel of complementary capture probes that has been developed for whole exome sequencing. This method differs from traditional RNA-seq sample preparation in that there is no poly-A selection. Instead, enrichment is generally done after the main enzymatic steps of library construction and a subset of PCR cycles. Unique to these approaches is a capture reaction (RNA-DNA hybridization) using exon-targeting RNA probes, followed by a washing step, and an additional set of PCR cycles. A motivation for utilizing such an approach with the systems and methods disclosed herein is the observation that coverage of splice junctions is quite high when utilizing a capture library step. There are a number of commercial sources for whole exome sequencing kits that can be used in the capture reaction of this approach such as Integrated DNA Technologies' (IDT) xGen Exome Research Panel v2 (Coralville, Iowa); Qiagen's QIAseq Human Exome Kits (Venlo, Netherlands); and Agilent's SureSelect Human All Exon (Santa Clara, Calif.).

Data produced by the sequencers are produced in a format called Binary Base Call (BCL). BCL files are stored in binary format and represent raw data output of a sequencing run. Ultimately, the BCL file is converted for use and storage in a format called FASTQ. This is a text-based format for storing both a biological sequence, in this case a nucleotide sequence and its corresponding quality scores, see Cock et al. Nuc. Acids Res. 38(6): 1767-71 (2009). As is well known to one of ordinary skill, the sequence letter and quality scores are each encoded using a single ASCII character for brevity. Although originally developed by the Wellcome Trust Sanger Institute to bundle FASTA format sequence data with quality information, it is now the de facto standard for storing the output of high-throughput sequencing instruments. As such, it is contemplated in embodiments of the systems and methods disclosed herein that this standard format will generally be used for the RNA-seq output files.

### B. Bioinformatics

The detection of disease relevant splice alterations is not trivial, as there are hundreds of thousands of annotated splice sites in the human genome. In addition, there is also great potential for the emergence of novel unannotated splice sites at countless locations in the genome. This suggests a need for robust statistical methods for detecting and quantifying differential splice events in comparative studies in health and disease.

Alternative splicing analysis consists of three main steps: detection, statistical comparison, and effect prediction. Software packages for detecting splicing alterations may be broadly broken down into two categories: those that only identify events found in annotated transcripts and those that additionally detect novel splice events. As aberrant splicing in disease states may result in novel transcripts, identifying novel splice events is desirable and an aspect of embodiments of the systems and methods disclosed herein.

Event and isoform detection and quantification are dependent on the correct assignment of RNA-seq reads to the molecule of origin. Thus, obtaining principal isoform input files from an annotation database for all expressed genes to act as a molecule of origin is an initial step in the present method. In this way, optional quantification of expression levels of genes from RNA-seq data may be done by mapping reads to the isoform input files and then counting mapped reads to each gene. Gene annotation data, which include chromosomal coordinates of exons for tens of thousands of genes, are required for this quantification process. There are several major sources of gene annotations that can be used for quantification, such as Ensembl and RefSeq databases. The process of counting mapped reads to genes requires a database of known genes. A gene is only quantified if it or its components have genomic coordinates already defined with respect to the genome sequence in a process called annotation. For each genome annotation model, a different set of annotation techniques and information sources are used and as such, these annotations vary in terms of comprehensiveness and accuracy of annotated genomic features.

Annotation techniques often include computer-based predictions and/or evidence-based techniques such as manual curation. Computer-based predictions can result in more complex gene models that have a higher proportion of predictive genomic features while evidence-based generated gene models may be simpler with fewer genes and isoforms. Common annotation models for human and mouse genomes include Ensembl, RefSeq, GENCODE, and UCSC annotations and any or all of these annotation databases can be used in the systems and methods disclosed herein. Annotations are, therefore, an important component in an RNA-seq analysis as the results may be affected by what is known in the annotation database. Further, an aspect of the present systems and methods disclosed herein is updating an annotation database with previously unidentified or undocumented variants with those found through the present methods. Although experimental work has been done to show that the choice of a particular annotation as an input source may impact experimental results (see, Chisanga et al., bioRxiv, https://doi.org/10.1101/2021.01.07.425794 (2021)), it is anticipated that the choice of a particular annotation source for principal isoform input files is within the scope of one of ordinary skill and may vary depending on the ultimate experimental goals for the alternative splicing identification. Further, normalization of data between various annotation sources can be utilized if change to a different annotation database is required (see, Chisanga et al., supra).

In particular, the annotation source is used to produce a principal isoform input file for each expressed gene from the patient sample or other cellular source such as an organoid. Principal splicing isoforms are determined through comparison to the constitutive RNA splicing method and the resulting protein products. An example of such an annotation database source for splice variants is APPRIS (see, Rodriguez et al. Nucleic Acids Res. 41(D1):D110-D117 (2013)). Although less comprehensive than APPRIS, other more general databases such as UniProt (see, The UniProt Consortium, Nucl. Acids Res., 49(D1): D480-D489 (2021)) or those associated with the National Center for Biotechnology Information (NCBI) (see, The NCBI Handbook, 2nd ed. (2013)) can also be utilized for data helpful for determining principal splicing isoforms. In addition to collecting, integrating and analyzing reliable predictions of the effect of splicing events, a primary advantage to APPRIS is that this database also selects a single reference sequence for each gene, here termed the principal RNA isoform reference sequence, based on the annotations of structure, function and conservation for each transcript. APPRIS identifies a principal isoform for 85% of the protein-coding genes in the GENCODE 7 release for ENSEMBL. Analysis of the APPRIS data shows that at least 70% of the alternative (non-principal) variants would lose important functional or structural information relative to the principal isoform. A useful feature of APPRIS is that it selects a principal isoform for each gene based on the reliable annotations for protein structure, function and cross-species conservation. The principal isoform is the representative isoform of the gene, the isoform against which all other (alternative) isoforms may be compared in various embodiments of the systems and methods disclosed herein. In APPRIS, the principal isoform is the isoform with the main cellular function, the isoform that is expressed in the majority tissues or in most stages of development or the isoform that is the most evolutionary conserved. Other criteria for designating an isoform as a principal isoform may be designed or chosen by one skilled in the art.

APPRIS comprises eight modules, as follows. It is anticipated that one of ordinary skill could select which combination of the modules of the database that would be effective as the sources of principal isoform files for the goals of their particular analysis. For example, Matador3D checks for the presence of structural homologs in the PDB and tests the integrity of the 3D structure; firestar makes highly reliable predictions of conserved functionally important amino acid residues; SPADE uses the program Pfamscan to count conserved and compromised Pfam functional domains; INERTIA uses three alignment methods to generate cross-species alignments, from which SLR identifies exons with unusual evolutionary rates; CRASH makes conservative predictions of signal peptides using the SignalP and TargetP programs; THUMP generates conservative predictions of trans-membrane helices from three separate trans-membrane predictors; CExonic uses exonerate to align mouse and human transcripts and looks for patterns of conservation in exonic structure and CORSAIR uses BLAST to map vertebrate orthologs to each variant and counts the numbers of orthologs that align correctly and without gaps. All of these methods are available as web services. Further, it has been established that APPRIS predictions are quite accurate, with agreement between the main proteomics isoform, the APPRIS principal isoforms, and the unique consensus coding DNA sequence (CCDS) variants was almost perfect (99.4%) over the 3015 genes where all three methods had a single reference isoform (see, M. Gonzalez-Porta, et al., Genome Biol., 14 (2013)). The fact that three entirely orthogonal sources of reference isoforms have such an outstanding agreement highlights the biological significance of the results from the proteomics experiments and significantly reinforces the likelihood that the main proteomics isoform is the dominant protein isoform in the cell (see Tress et al., Trends Biolog. Sci., 42(2):98-100 (2017).)

It is also at this point in the embodiments of the present methods that target isoform files can be provided. Target isoform files are optional forms of genes of interest, that will generally not be equivalent to the principal splicing isoform of the gene. Particularly when not equivalent to the principal splicing isoform, this target isoform, if known in advance, can be added to the set of isoforms that will be compared to the RNA-seq reads. This is done, in one embodiment of the present method, through describing the target sequence(s) and feeding such sequences into the comparison pipeline using a custom Javascript Object Notation (json) file, although other implementations would be well known to one of ordinary skill. Without being bound by theory, such target isoforms are anticipated to be those forms of splicing events that have been previously associated with or are suspected to have particular biological relevance. This previously identified or suspected biological relevance may be association with a particular disease, see for example Wu et al., Oncogen, 40: 4184-4197 (2021), which discusses biological relevance of alternative splicing events in esophageal squamous cell cancer, or Xiong et al., Front. Genet. 11: 879 (2020) which discusses the same in the context of hepatocellular carcinoma.

Importantly, such target isoforms may not themselves have been previously identified as associated with disease, but simply encompass all known or predicted splicing isoforms of a particular gene of interest, where the gene or collection of genes is therefore the level of identified biological relevance. The use of target isoform files in the embodiments of the present method is entirely optional, as identification of such newly identified and documented splicing isoforms for further investigation is a primary goal of the present method. But if the ultimate goal of the use of the method includes the investigation of a known or predicted or identified alternative splicing event, where such knowledge or prediction or identification occurs before the performance of the present method, the method is equally useful in providing such information. Such identification and quantification of target events can thereafter become a part of the produced splicing report for a particular patient or patient set, as discussed more extensively below.

However, although this is not required, many target isoforms will be encoding genes which have been previously identified to be associated with disease and have previously been identified to have splicing variants in disease states. The disease states can be associated with mutations present in the genes that have been shown to cause the splicing variants. Such genes have been identified in the art, see for example, the genes and the isoforms discussed in Scotti & Swanson, Nat. Rev. Genet., 17: 19-32 (2015); Abramowicz & Monika, J. Appl. Genet. 59(3):253-268; and Sahakyan & Balasubramanian, BMC Genomics, 17: 225(2016). In particular, Abramowicz & Monika discuss the MIP gene, involved in Autosomal dominant congenital cataracts, the NF1 gene, involved in Neurofibromatosis type 1; the COL5A2 gene, involved in Ehlers-Danlos syndrome; the OXCT1 gene, involved in Succinyl-CoA:3ketoacid CoA transferase (SCOT) deficiency; the DMD gene, involved in Becker muscular dystrophy (BMD); the ELP1(1KBKAP) gene, involved in Familial dysautonomia (FD); the CFTR gene, involved in Cystic fibrosis (CF); the AR gene involved in Androgen insensitivity syndrome; the GLA gene, involved in Fabry disease; the DMD gene, involved in Duchenne muscular dystrophy (DMD); the TRAPPC2 gene, involved in X-linked spondyloepiphyseal dysplasia tarda; the ACADM (MCAD) gene, involved in Medium-chain acyl-CoA dehydrogenase (MCAD) deficiency; the COL2A1 gene, involved in Stickler syndrome; the XPC gene, involved in Xeroderma pigmentosum; the F9 gene, involved in Hemophilia B; and the ACAT gene, involved in Mitochondrial acetoacetyl-CoA thiolase (T2) deficiency. Any of the genes disclosed in these references could be suitable sources for a target isoform. For further non-limiting particular examples, the systems and methods disclosed herein encompasses the use of isoforms of EGFR, AR, MET, NOTCH1, NOTCH2, NOTCH3, and NOTCH4 as possible target isoforms.

Because principal splicing isoform or target isoforms can be derived from multiple annotation databases, embodiments of the present methods further encompass an optional pre-processing step that removes inconsistent annotation and provides a standard or consistent labeling approach for all the principal or target isoforms that are to be used in the upcoming steps of the present method. Adoption of a consistent file format and labeling of contents of that file is anticipated to be optionally necessary and at the same time, well within the skill set of one of ordinary skill in the bioinformatics arts. Consistency is needed in naming conventions, documentation and expression of start and stop sites of transcription in comparison to genomic sequences, and other documentation related labels in order to ensure that the matching process will be the same no matter what is the initial database source of the principal splicing isoform or target isoform.

Once the principal isoform input files for the selected expressed genes are established and any desired target isoforms are added and made consistent, if needed, a read mapper that is splice-site aware, and therefore, can be used to detect exon-intron boundaries and connections between exons is used for the next step in embodiments of the systems and methods disclosed herein. In various embodiments, all expressed genes are selected. In other embodiments, a portion of the expressed genes are selected. The alignment of RNA-seq reads to reference files, such as the principal isoform input files of the systems and methods disclosed herein, have been addressed in the past by tools that combine fast heuristics for sequence matching with a model for splice-sites. These methods, however, are generally not competitive enough to map all reads from a sequencing run in a reasonable time. A myriad of alternative methods have therefore been developed for mapping short reads to a reference genome (see, Fonseca et al., Bioinformatics 28(24):3169-772012 (2012); Engström et al., Nature Methods, 10:1185-1191 (2013) for representative reviews). Those that are splice-site aware and incorporate intron-like gaps are generally called spliced-mappers, split-mappers, or spliced aligners. Their main challenge is that reads must be split into shorter pieces, which may be harder to map unambiguously; and although introns are marked by splice-site signals, these occur frequently by chance in the genome. It is these spliced-mappers, split-mappers, or spliced aligner tools that can function to map the principal isoform input files to the RNA-seq reads in an embodiment of the present method. It is anticipated that multiple different mapping tools could be successfully utilized in the embodiments of the present method.

A representative aligner for use in the present methods is Spliced Transcripts Alignment to a Reference (STAR) software. This software utilizes a specially developed RNA-seq alignment algorithm, see Dobin et al, Bioinformatics, 29(1):15-21 (2013), that allows for relatively high speed alignment of reads to reference sequences, such as the human genome, with high precision. Briefly, the algorithm accomplishes this by utilizing a sequential maximum mappable seed search in uncompressed suffix arrays followed by seed clustering and stitching procedures. The seed searching step involves a sequential search for a Maximal Mappable Prefix (MMP). MMP is similar to the Maximal Exact (Unique) Match concept used by the large-scale genome alignment tools Mummer and MAUVE. Given a read sequence R, read location i and a reference genome sequence G, the MMP(R,i,G) is defined as the longest substring (Ri, Ri+1, . . . , Ri+MML−1) that matches exactly one or more substrings of G, where MML is the maximum mappable length. This approach represents a natural way of finding precise locations of splice junctions in a read sequence and is advantageous over an arbitrary splitting of read sequences used in split-read methods. The splice junctions are detected in a single alignment pass without any a priori knowledge of splice junctions' loci or properties, and without a preliminary contiguous alignment pass needed by the junction database approaches, thus making it a very useful alignment tool for embodiments of the systems and methods disclosed herein. The MMP in STAR search is implemented through uncompressed suffix arrays (SAs) which provides both efficiency and speed, although there is increased memory usage as compared to compressed SAs.

In the second phase of the algorithm, STAR builds alignments of the entire read sequence by stitching together all the seeds that were aligned to the reference files, such as the principal splice isoforms or target splice isoforms, in the first phase. First, the seeds are clustered together by proximity to a selected set of ‘anchor’ seeds. All the seeds that map within user-defined genomic windows around the anchors are stitched together assuming a local linear transcription model. The size of the genomic windows determines the maximum intron size for the spliced alignments. A frugal dynamic programming algorithm is used to stitch each pair of seeds, allowing for any number of mismatches but only one insertion or deletion (gap). Importantly, the seeds from the mates of paired-end RNA-seq reads are clustered and stitched concurrently, with each paired-end read represented as a single sequence, allowing for a possible genomic gap or overlap between the inner ends of the mates. This is a principled way to use the paired-end information, as it reflects better the nature of the paired-end reads, namely, the fact that the mates are pieces (ends) of the same sequence. This approach increases the sensitivity of the algorithm, as only one correct anchor from one of the mates is sufficient to accurately align the entire read.

If an alignment within one genomic window does not cover the entire read sequence, STAR will try to find two or more windows that cover the entire read, resulting in a chimeric alignment, with different parts of the read mapping to distal genomic loci, or different chromosomes, or different strands. STAR can find chimeric alignments in which the mates are chimeric to each other, with a chimeric junction located in the unsequenced portion of the RNA molecule between two mates. STAR can also find chimeric alignments in which one or both mates are internally chimerically aligned, thus pinpointing the precise location of the chimeric junction in the reference files.

The stitching is guided by a local alignment scoring scheme, with user-defined scores (penalties) for matches, mismatches, insertions, deletions and splice junction gaps, allowing for a quantitative assessment of the alignment qualities and ranks. The present method commonly utilizes the default parameters, which includes, most importantly a maximum intron length of 1 Mbp, as 100 kb value was found to be shorter than the intron between the splice sites of interest. By utilizing the lMkp value, most annotated introns in the human genome can be captured, and therefore, also the novel ones. These parameters have also been most carefully evaluated by the ENCODE consortium for valid applicability. But it is anticipated that these values could be altered from the default set in certain embodiments of the present method, to accommodate particular analysis goals. The stitched combination with the highest score is chosen as the best alignment of a read. For multimapping reads, all alignments with scores within a certain user-defined range below the highest score are reported.

Although the sequential MMP search only finds the seeds exactly matching the genome, the subsequent stitching procedure is capable of aligning reads with a large number of mismatches, indels and splice junctions, scalable with the read length. This characteristic has become ever more important with the emergence of the third-generation sequencing technologies that produce longer reads with elevated error rates. Such third generation sequencing technologies are anticipated to be possible sources of RNA-seq reads for use in certain embodiments of the present method. The algorithm extensibility to long reads shows that STAR can potentially serve as a universal alignment tool across a broad spectrum of emerging sequencing platforms. STAR can align reads in a continuous streaming mode, which makes it compatible with advanced sequencing technologies such as nanopore sequencing (Oxford Nanopore Technologies, Oxford, UK). These characteristics, plus the general functionality of the STAR aligner tool, makes it very useful for embodiments of the systems and methods disclosed herein, given the variety of anticipated sources for the RNA-seq data, but as discussed above, other alignment tools that are splice site aware, such as a pseudo-aligner like Kallisto, see Bray et al., Nat. Biotechnol. 34(5):525-7 (2016), can also be utilized. It is noted that one of ordinary skill would be aware of the means to determine which alignment tool would be most effective for the specific goals of the present method, such as utilizing the tool CABURE in order to evaluate the effectiveness of the alignment produced by various alignment methods, see Kumar, et al., Sci. Rep. 5:13443 (2015).

In embodiments of the systems and methods disclosed herein, the output of the STAR aligner is utilized to compare the RNA-seq reads from the patient sample to the principal splicing isoform files and/or target isoform files. If a selected number of RNA-seq reads have a splice junction pattern differing from the principal splicing isoform, it is identified as a novel splice pattern. If a selected number of RNA-seq reads match splice junctions from a target isoform, it is identified as detection of a target event. Importantly, both comparisons to principal isoform files and target isoforms files occurs during the same comparison process. The exact number of events that are needed to record the result as a novel splice pattern or as a detection of a target event can vary depending on the experimental goals of the performance of the present methods, however, one possible embodiment of the present method involves the need to detect at least about 5, 10, 15, 20, 25, or 30 reads with the novel splice pattern, or match to the target splice pattern before it is reported as available for further analysis. As can be appreciated by one of ordinary skill, the selection of the appropriate read number can be informed or filtered by other values, such as percent spliced in index (PSI) discussed more fully below.

Target events that are detected do not undergo further analysis, but are instead quantitatively provided directly to the output table for inclusion in the general report or the specialized patient splicing event report, depending on the final desired outcome of the present methods. However, splicing graphs for such events can be optionally generated. Novel splice patterns, in contrast, undergo significant further analysis to allow for categorization and documentation of the detected event(s). In particular, novel splice patterns are further categorized as to the type of alternative splicing event that has occurred in the identified reads, determined based on the type of differences between the identified splice junction pair and the principal isoform. For example, if a detected splice junction is linking two splice sites from the principal splicing isoform but from two non-consecutive exons, this is identified and recorded as a novel exon skipping event. These events are preferably detected in each sample individually and evidence for their existence is only present upon detection, so if there is no read supporting a given exon skipping event in a patient sample, that exon skipping event, although theoretically possible, is not present in the final output table.

A second type of event that can be found is a detection of a novel exon. These are defined as any exon detected with one or more splice sites not being present in the principal splicing isoform for the gene. They are detected by combining splice junction information with heuristic analysis. The process of novel exon detection can be summarized in the following steps: (1) selection of novel splice junctions, defined as splice junctions connecting one splice site in the principal isoform transcript to a splice site not in the principal isoform transcript, or connecting two splice sites that are not in the principal isoform transcript followed by (2) matching novel acceptor sites to novel or known donor sites within a certain genomic range to build the novel exon, or similarly, matching novel donor sites to novel or known acceptor sites within a certain genomic range to identify the involved sequence and thereafter build the map or other documentation of the identified novel exon. These certain genomic ranges can comprise a minimum and maximum genetic distance from the first unmatched splice site to define the range in which the exon sequence is searched for, for example about 10 to about 1500 bp. If a novel splice site cannot be matched to any other splice site within the defined genomic ranges, it is considered the splice site of a terminal (that is, 3′) exon. A useful aspect of an embodiment of the systems and methods disclosed herein is the creation of nomenclature for novel exons, for example, if a novel exon is identified that is now determined to comprise sequence that is between previously annotated exons 1 and exons 2, such new exon will be documented with the name exon 1b or exon 1.5. Further, combinations of known and unknown splice sites are also utilized for a full annotation of the newly discovered exon boundaries. This annotation may include chromosomal locations or other information that indicates where the exon boundary is located in a genome.

The file format produced by the aligner tool can be initially present in a sequence alignment map (SAM) format. This is a text-based format that is generic, can support short and long read alignments produced by a variety of different sequencing platforms, and is human-readable. This format was developed specifically for storing biological sequences aligned to a reference, see Li et al., Bioinformatics, 25(16):2078-9 (2009). However, it is anticipated that this format is not the most efficient for the subsequent analyses that may be needed for particular alignments, thus conversion to a binary alignment map (BAM) format is contemplated in embodiments of the systems and methods disclosed herein. This is a lossless, compressed binary representation of the SAM files and was developed by Li et al. to be used in conjunction with SAM files. The advantages of working with BAM files include smaller file size and resulting speed during analysis. However, these files are only machine readable and are not generally utilized for human directed output. The usefulness of conversion between SAM to BAM (or in reverse, if needed) are well known to one of ordinary skill. For example, the STAR alignment system can convert SAM files into BAM files as part of its standard output protocol. Embodiments of the systems and methods disclosed herein utilize this standard approach in producing BAM files that will be utilized for further analysis as described previously. Finally, a further file format called compressed columnar (CRAM) has also been developed, through some file restructuring, to store such data using even less storage space, see Fritz et al., Genom. Res. 21 (5):734-40 (2011). This format could also be utilized within embodiments of the systems and methods disclosed herein.

One issue that can be encountered in the systems and methods disclosed herein is the existence of exons with overlapping splice sites. These are cases where, for example, multiple detected exons have the same acceptor site, but multiple donor sites, or alternatively, multiple detected exons can have the same donor site, but multiple acceptor sites. Usually, one combination of splice sites is the most predominant one, in terms of read counts, but the present method aims to keep as many combinations as possible in order to detect low-abundance isoforms, without overly increasing computing complexity. In this regard, a minor but not infrequent proportion of samples have one to two genes that have many alternative splicing events, and keeping all combinations of novel exons makes the computations increase exponentially. In some embodiments, it is known that particular genes have this issue in the context of the sample to be analyzed and thus the filter can be applied at the beginning of the analysis, while in others, this filter will become necessary based on the computational complexity that results in the method without the use of the filter. To address this issue, the systems and methods disclosed herein can optionally include an additional filter on exons with overlapping splice sites. The filter is only applied to splice sites that are shared by more than a user-defined number of exon combinations, such as about 50. However, this user-defined number of exon combinations can be as few as about 10, 20, 30, 40, and as many as about 50, 60, 70, 80, 90 or 100.

In particular, one method of filter that has proven effective is the maintenance of exon combinations that are supported by a number of reads that is higher than the median cumulative number of reads for that splice site. In other words, for a given splice with more than a set number of combinations, the number of reads are sorted that support each combination, and applying the cumulative sum, the number of reads that split the cumulative sum in half is identified and used as a threshold to select only the most abundant combinations of splice sites. As would be evident to one of ordinary skill, this method of filter is one of many such user-defined possibilities. In general, the means of implementing the optional filter should be governed by some sort of numeric cut off as to detected reads within a particular overlap of exon splice sites. It is anticipated that this process, which is necessary in particular cases to reduce computational complexity to a reasonable level, may involve the elimination of read-based artifacts that can be considered false positives for alternative splicing variants. However, whatever these reads may represent, elimination of them from the post-alignment calculations is an optional step in practical application of embodiments of the present method.

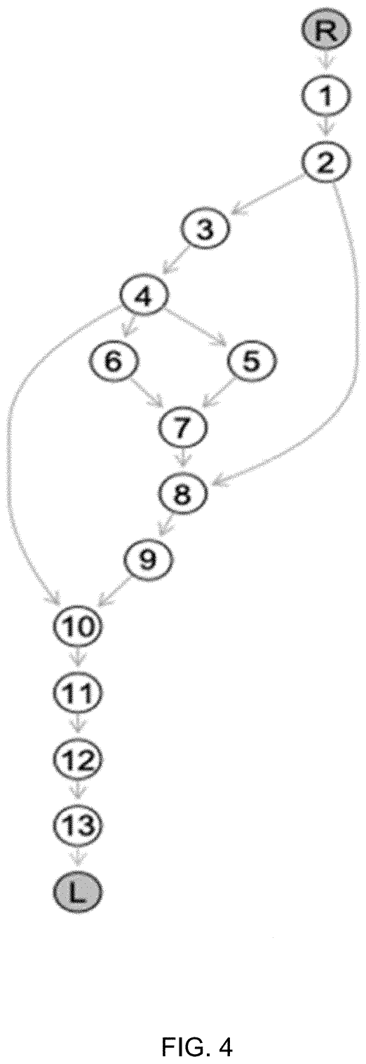

The next step in certain embodiments of the present method is the building and documentation of all identified alternative splice variants into alternative splicing transcripts. All or a selected subset, if only certain types of alternative splice events are of interest, of these alternative splice variants can be deposited into a isoform dictionary, which holds the sequences and other related documentation of those alternative splicing events that have been identified using the prior steps of the present method. A primary use of this provided isoform dictionary is to utilize its entries to build splicing graphs for the identified alternative splicing events. Splicing graphs are a convenient representation of all identified splicing variants for a particular gene. It differs from other representations of splicing variants as it does not utilize a linear sequence-based approach but instead uses a graphic representation where each identified splice variant is a path on the graph. A representative example of a splicing graph is provided in FIG. 4.

Briefly, splicing graphs represent the following conditions. Let {s1, . . . , sn} be the set of all RNA transcripts for a given gene of interest. Each transcript si corresponds to a set of genomic positions Vi with Vi≠Vj for i≠j. Define the set of all transcribed positions Vi=Uni=1 Vi as the union of all sets Vi. The splicing graph G is the directed graph on the set of transcribed positions V that contains an edge (v,w) if and only if v and w are consecutive positions in one of the transcripts si. Every transcript si can be viewed as a path in the splicing graph G and the whole graph G is the union of n such paths. Vertices with indegree=outdegree=1 can be collapsed to obtain a more compact representation of the splicing graph. Splicing graphs are similar to gene models that represent exons connected by edges if they are consecutive in a transcript. However, in contrast to gene models, splicing graphs can be built solely from transcript data without any knowledge of the genomic sequence, see Heber et al., Bioinformatics, 18(S1):S181-S188 (2002) for an introduction.

A very useful tool for the building of splice graphs involving alternative splicing (AS) variants is the alternative splicing transcriptional landscape visualization tool (ASTALAVISTA), see Foissac and Sammeth, Nucl. Acids Res., 35 (Web Server issue):W297-9 (2007), which is available both as a local or web-server based application. In brief, given a set of annotated transcripts, the method consists in first considering all pairwise comparisons between overlapping transcripts. A variation of the splicing structure is detected if some splice sites are not used in both transcripts. Then, according to the genomic coordinates, the relative order of the splice sites that are included in such variations is used to build a code describing the corresponding alternative splicing event. From the provided annotation with respect to the specified options, the ASTALAVISTA protocol dynamically extracts AS events. As a summary, the main result page shows a list where each event type is depicted in the relative-position notation is given. The list is ranked according to the occurrence (number or proportion) of the events. A graphical overview is provided in the form of a pie diagram that displays the distribution of events across the groups, considering differentially each type of simple event and pooling the others in one group. In particular, the alternative splicing landscape is described by a list of alternative splicing events grouped according to equal variations in the exon-intron structure between transcripts. A schematic picture illustrates every type of event, specified by the respective code in the relative splice site position notation. The list is ranked according to the observed frequency of events, and as an overview, a pie diagram shows the resulting distribution. For each type of AS event, the enumeration of all genes/transcripts involved is provided, including the corresponding identifiers and genomic coordinates. The genomic positions are dynamically linked to the UCSC genome browser for further analysis. This tool can be utilized on the isoform dictionary to provide graphical representations of the various alternative splicing events of interest that are present in the dictionary for one or more particular genes. However, as is well known to those of ordinary skill in the art, other methods of graphically representing the isoforms can also be utilized. The ultimate output of this possible method step is a further json file which utilizes a format called General Transfer Format (.gtf). These generated splicing graphs can be useful for identifying and recording the quantification of the number of reads present in a particular sample for each of identified, graphed, and documented alternative splicing events.

A further possible step in certain embodiments of the present method is the computation of the percent spliced in index (PSI) for each exon of interest. In its most basic sense, PSI is the ratio between the number of reads including (or excluding) exons and the total number of reads, see Schafer et al., Curr. Protoc. Hum. Genet. 87:11.16.1-11.16.14 (2015). This value is believed to represent how efficiently the examined exons are spliced into (or spliced out of) transcripts and can be utilized to provide a full picture of the alternative splicing occurring at a genetic locus. It was developed to allow visualization of alternative splicing in an exon-centric manner and can be used to compare alternative splicing across medical conditions. In embodiments of systems and methods disclosed herein, this calculation has been adapted to apply to all types of alternative splicing events that can be detected. For example, for single exon skipping, inclusion reads are multiplied by two because one exclusion read actually spans both splice junctions of the missing exons. For each alternative splicing event in a given sample, its PSI value is estimated by the proportion of exon-exon junction read counts supporting the inclusion isoform.

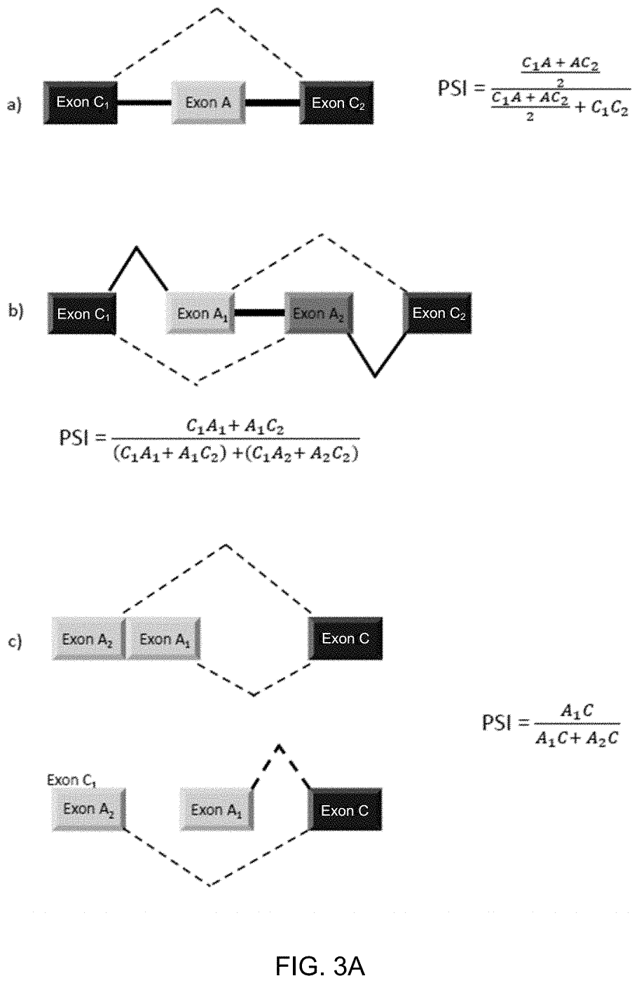

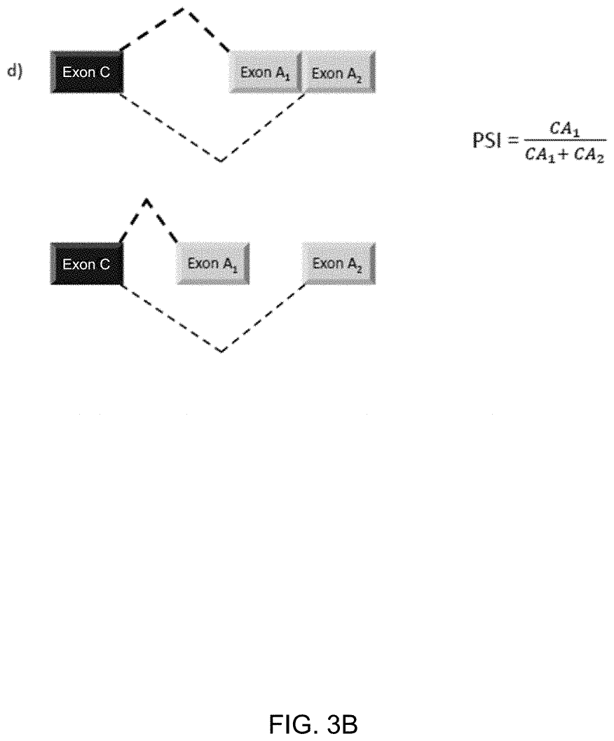

As mentioned above, the junction reads required for alternative splicing quantification depend on the type of event, see Saraiva-Agostinho and Barbosa-Morais, Nucl. Acids Res. 47(2):e7 (2018) and also, FIG. 3, derived therefrom. In this figure, C1A and AC2 represent read counts supporting junctions between a constitutive (C1 or C2, respectively) and an alternative (A) exon and therefore alternative exon A inclusion, while C1C2 represents read counts supporting the junction between the two constitutive exons and therefore alternative exon A exclusion. Alternative splicing events involving a sum of junction read counts supporting inclusion and exclusion of the alternative sequence below a user-defined threshold (10 by default, for example) can be discarded to avoid imprecise quantifications based on insufficient evidence. Once the specific alternative splicing quantification is performed, that is, the PSI or other related numeric representation of the relative amount of alternative splicing is calculated, this value can be provided as part of the output of the present method, either on its own or as part of a collection of data in a report.

A further possible analysis performed by embodiments of the systems and methods disclosed herein is the comparative analysis of novel skipped, novel added, or novel terminal exons to the protein structure of the encoded gene. This analysis can be done to determine if there is a possible functional difference in the protein that would be encoded by the novel splice variant compared to the protein that is encoded by the principal RNA isoform reference sequence. Such analysis is known in the art and has been described, for example, in Foris et al., BMC Genom. 453 (2008); Heygi et al., Nucleic Acids Res., 39(4): 1208-1219 (2011). Briefly, some of the analyses that can be performed are (i) analysis of the impact of truncated or inserted domains, (ii) calculation of intrinsic protein disorder that results from the splicing variant; and (iii) analysis of newly exposed surfaces, particularly those with hydrophobic properties, on the protein resulting from the splice variant. A further example of possible categorization of protein function impacts associated with splice variants is described by Ferrer-Bonsoms et al., Scientific Reports, 10 (1069) (2020). This group constructed a web application that can predict the impact on protein function of various splice variants.

The reports provided by embodiments of the systems and methods disclosed herein are anticipated to comprise one or more of identifications of alternative splicing variants. Each alternative splicing event, particularly those that have not previously been identified in splicing annotations, is provided with a splicing event identifier. Such identifiers will be used consistently across multiple patient sample reports where the same variant is found. Further data can be included such as the number of RNA-seq reads that support one or more of the identified alternative splice variants, representations of the variants such as splicing graphs, and relative amount of alternative splicing calculations, such as the PSI or such similar calculations for the type of alternative splicing variants identified. In particular, a possible report within the systems and methods disclosed herein could include, for one or more alternative splicing variants, one or more of the following fields: splicing event identifier, the gene name, alternative splicing coordinates, event description (e.g. type of alternative splicing event); domain overlap of the splicing event with the encoded protein; other genetic characteristics and the number of reads that support the identified alternative splicing event described.

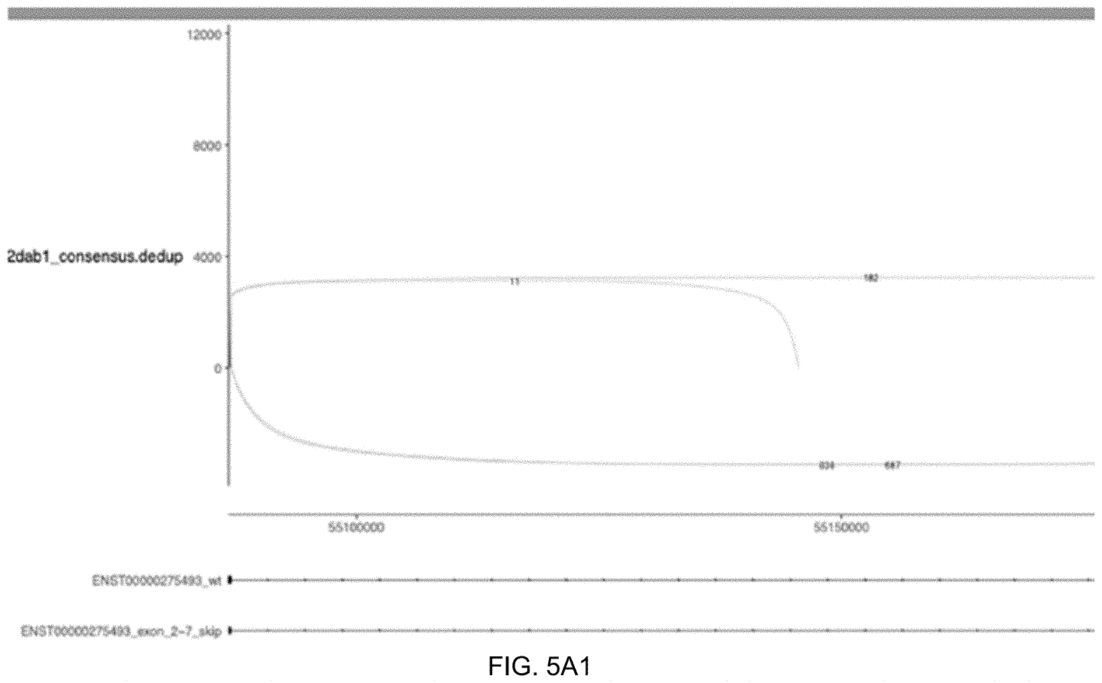

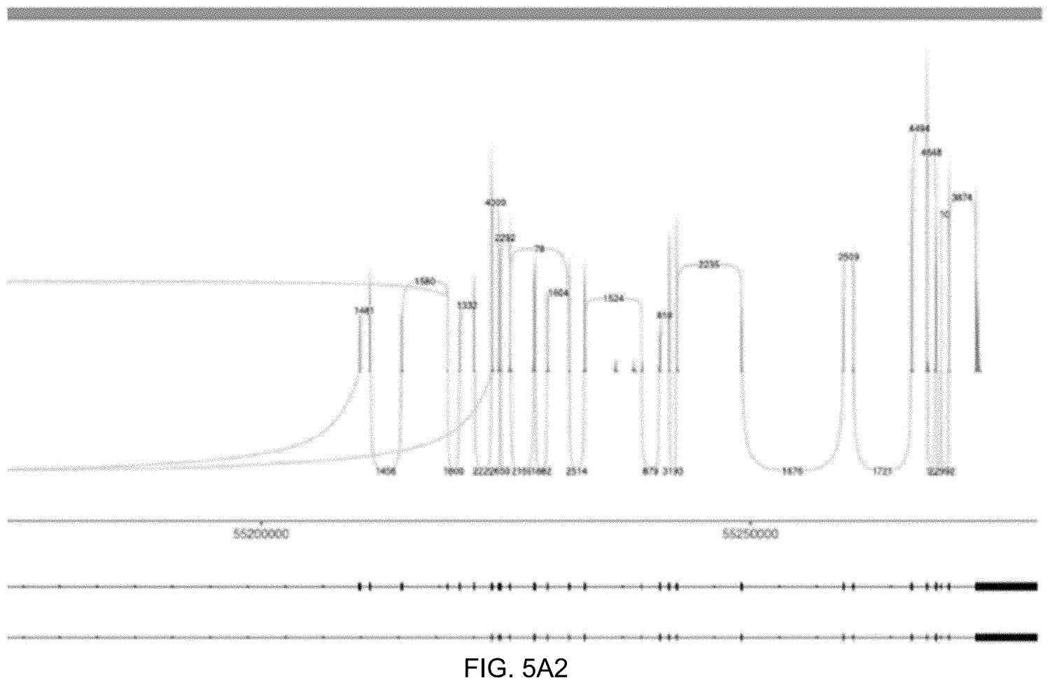

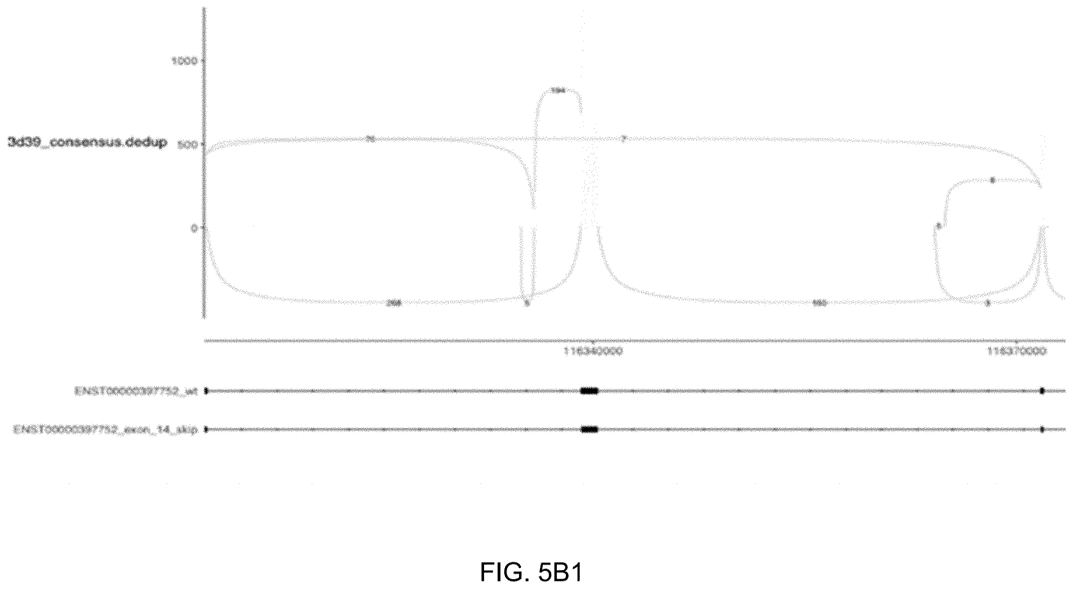

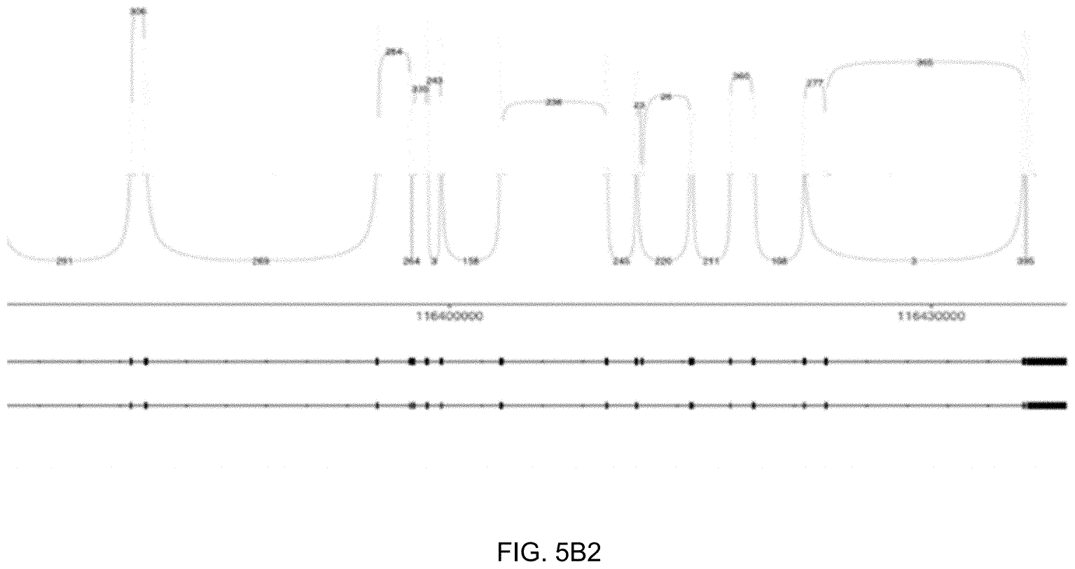

Optionally, the report can include a graphic representation of one or more of the alternative splicing variants. Although many such graphical representations could be useful, one particularly useful one is a Sashimi plot, see, Katz et al., arXiv, 1306.3466v1 (2013). Sashimi plots are made using gene model annotations along with read alignments to generate a quantitative summary of the genomic and splice junction reads. Two exemplary Sashimi plots are provided in FIGS. 5A and 5B. Genomic reads are converted into read densities (per base) scaled by the number of mapped reads in the sample, measured in RPKM units. Splice junction reads are plotted as arcs whose width is proportional to the number of junction reads that span the exons connected by the arc. Sashimi plots require two main inputs, (1) Alignments of reads to the genome (including junctions), provided in the standard BAM format. Read mappers that produce splice junction alignments, such as STAR, produce these; and (2) annotation of gene models or alternatively spliced events in GFF3 format (GFF). These annotations can be downloaded from databases such as Ensembl or UCSC, or custom-generated (e.g. based on de novo transcript assembly programs). Alternative isoform annotations in commonly studied genomes (such as those available from the MISO website) can be optionally used with Sashimi plots. A third optional input includes quantitative estimates of isoform abundance (Ψ values), as estimated by MISO, which can be displayed alongside the Sashimi plots.

The report can further include therapies or clinical trials associated with at least a portion of the alternative splice variant information included in the report. For example, a report having a splice variant detected in a MET gene, may further include a ET inhibitor and information indicating that the ET inhibitor therapy may be a therapeutic option for a patient having the MET splice variant. Examples of MET inhibitors include capmatinib or tepotinib. The report can also include control data, that is the constitutive RNA splicing events, the amount generally seen of these constitutive RNA splicing events, or other information that is found in non-patient, control specimens. It is anticipated that a report generated by the systems and methods disclosed herein will be useful for either clinicians or researchers to guide future decision-making in the patient therapy, research directions, or other related areas.

Table 1 provides the fields, descriptions, and proposed variable type utilized in an example report for an embodiment of the systems and methods disclosed herein.

TABLE 1

| Field | Description | Proposed type |
|---|---|---|
| needs_review | True when the event needs clinical science review, | bool |
| | False otherwise. Only events in reportable genes need | |
| | clinical science review | |
| reportable_gene | True when the gene is reportable, False otherwise | bool |
| gene_name | Gene name | varchar(1000) |
| domain | Domain overlapping the event, if any | varchar(1000) |
| exon_exclusion_read_support | Number of reads supporting exon exclusion | int(11) |
| exon_inclusion_read_support | Number of reads supporting exon inclusion | int(11) |
| psi | Percent spliced-in. If 1 the exon is totally included, | float(5, 2) |
| | if 0 the exon is totally excluded. | |
| description | Description of the alternative splicing event. | varchar(1000) |
| start | Genomic position of the upstream flanking splice site. | int(11) |
| end | Genomic position of the downstream flanking splice | int(11) |
| | site. | |
| event_type | Type of alternative splicing event. ale: alternative | varchar(1000) |
| | last exon, afe: alternative first exon, mes: multi-exon | |
| | skipping, aes: alternative exon skipping, aa: alternative | |
| | acceptor, ad: alternative donor, ir: intron retention, mee: | |
| | mutually exclusive exons | |
| chr | Chromosome | varchar(1000) |
| event_start | Event start coordinate | int(11) |
| event_end | Event end coordinate | int(11) |
| domain_start | Domain start coordinate | int(11) |
| domain_end | Domain end coordinate | int(11) |
| gene_start | Gene start coordinate | int(11) |
| gene_end | Gene end coordinate | int(11) |
| transcript_id | Novel id assigned to the transcript produced by the | varchar(1000) |
| | alternative splicing event | |
| gene_id | Ensembl gene id | varchar(1000) |
| strand | Strand | varchar(1) |
| sashimi | True when the transcript appears in the sashimi plot | bool |
| wt_transcript_id | Ensembl transcript id of the wild type transcript | varchar(1000) |
| | (principal isoform). | |
| event_id | Unique id for the splicing event, obtained by hashing | varchar(1000) |
| | the coordinates of the involved splice sites | |
| source_analysis_id | Bioinformatics analysis id | varchar(1000) |

### C. Further Methods

Embodiments of the present methods also involve the building of a splice profile of alternative splice variants for a particular patient sample. In essence, the splice profile is a specific example of the report that can be provided in the systems and methods disclosed herein. The data that populates the splice profile is obtained using the similar comparing splice junction data from the patient sample to the principal RNA isoform for each gene within the RNA-seq reads from the patient sample; identifying those RNA-seq reads that describe novel exon skipping variants, novel exon addition variants, or novel terminal exon variants through said comparison; documenting at least one of the skipped exons, the added exons, or the terminal exons using a splicing graph for each alternative splicing variant including providing a fully annotated description and splice junction coordinates; optionally using the splicing graphs or some other documentation of the identified alternative splice variants to produce a patient sample specific isoform dictionary; providing the quantity of reads supporting each entry in the isoform dictionary; and building a report associating at least one of the isoform dictionary entries with sequence variant identifiers. The identifier will be utilized across multiple patient reports where the same variant is found, providing consistent identification and association of that splice variant with future measurements as they occur with different patients, such as therapeutic outcomes. This is particularly useful when the present method is the first identification and documentation of a novel splice variant.

Many splice profiles are likely to include all the alternative splice variants that were identified in the detection method, but this is not necessarily a requirement, depending on the ultimate use planned for the splice profile. The optional use of target sequence comparisons may be a common inclusion in splice profiles. Splice profiles will be of use for both clinical and research based decision-making. It is anticipated that all variations of the detection method can be utilized to equal utility in producing splice profiles for particular patients' samples. Adaption of the precise contents of the report section of the detection method is anticipated to be part of the splice profile method, and such adaption is believed to be well within the purview of one of ordinary skill, once the identification of alternative splice variants and calculation data concerning the relative frequency of those alternative splice variants are obtained. However, it should be emphasized that embodiments of the present method involving splice profiles aim to associate newly discovered splice variants with patient data, such as therapeutic response, therapeutic non-response, and overall clinical outcome. It is anticipated that splice variant reports, when backed up with multiple patient samples showing presence or absence of the same splice variants, will provide valuable input into clinical decision-making for diseases associated with such splice variant profiles.

The usefulness of the splice profile is that it provides quantitative basis for decisions such as providing data surrounding alternative splice variants that can be targeted by a therapy or drug; variants that are biomarkers for successful response to a therapy or drug; variants known to affect disease course or prognosis; or variants that can help with diagnosis. Further, the splice profile can merely consist of an overall picture of splicing in the patient or specimen, for example, merely addressing whether there are a greater number or a greater percentage of alternative splice variants compared to a typical specimen. Additionally, the splice profile can provide quantitative basis for decisions involved in research based decision-making such as alternative splice variants that can be targeted by a currently researched therapy or drug; variants that are being investigated as being biomarkers for successful response to a therapy or drug; variants that are being investigated as to affect disease course or prognosis; or variants that are being investigated as to usefulness for diagnosis. Further, at a research level, splice profile can merely consist of an overall picture of splicing in the patient or specimen, for example, merely addressing whether there are a greater number or a greater percentage of alternative splice variants in patients suffering from a particular disease as compared to a typical specimen.

A further embodiment of the present methods provides one exemplary use of the produced splice profile, namely the methods of developing a companion diagnostic test for a treatment method of a disease based on the presence or absence of alternative splicing variants in a patient sample. This method relies on two situations currently present. First, as discussed previously, there are a wide range of diseases associated with alternative splice variants, and as this is an active area of research, more and more diseases are being linked to such associations. Such biological impact of alternative splice variants provides strong motivation for the production of splice profiles for individual or groups of patient samples (see, for example, Truty et al., Am. J. Hum. Gene., 108: 696-708, 2021 discussing the frequency of alternative splice variants that can be predicted to be contributing to disease). Companion diagnostics are defined by the FDA as a device that “provides information that is essential for the safe and effective use of a corresponding drug or biological product,” companion diagnostics aim to help health care professionals determine whether the benefits of a specific therapy outweigh potential side effects or risks (see, Nalley, Oncology Times, 39(9):24-26, discussing the use of companion diagnostics in the oncology setting). Thus, embodiments of the systems and methods disclosed herein aim to provide information that can be associated with the safe and effective use of a corresponding drug.

These methods comprise the steps of preparing the splice profiles for a plurality of patients suffering from a disease; associating the treatment response of the patients to a particular treatment method for the disease; determining a further association between positive treatment responses and the presence or absence of particular alternative splice variants in the splice profile for the patient samples; and using the presence or absence of the particular alternative splice variants in a splice profile to identify further patients more likely to benefit from the treatment method than those patients without the presence or absence of the particular alternative splice variants in their splice profile, thus providing a companion diagnostic for the particular treatment method for the disease. One use of this method is when the disease is cancer.

Examples of cancer include, but are not limited to, carcinoma, lymphoma, blastoma, glioblastoma, sarcoma, and leukemia. Cancers may include, for example, breast cancer, squamous cell cancer, lung cancer (including small-cell lung cancer, non-small cell lung cancer (NSCLC), adenocarcinoma of the lung, and squamous carcinoma of the lung (e.g., squamous NSCLC)), various types of head and neck cancer (e.g., HNSC), cancer of the peritoneum, hepatocellular cancer, gastric or stomach cancer (including gastrointestinal cancer), pancreatic cancer, ovarian cancer, cervical cancer, liver cancer, bladder cancer, hepatoma, colon cancer, colorectal cancer, endometrial or uterine carcinoma, salivary gland carcinoma, kidney or renal cancer, liver cancer, prostate cancer, vulval cancer, thyroid cancer, and hepatic carcinoma, as well as B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's Macroglobulinemia), chronic lymphocytic leukemia (CLL), acute lymphoblastic leukemia (ALL), hairy cell leukemia, chronic myeloblastic leukemia, and post-transplant lymphoproliferative disorder (PTLD), as well as abnormal vascular proliferation associated with phakomatoses, edema (such as that associated with brain tumors), and Meigs' syndrome.

As would be well understood by one of ordinary skill, the term “cancer” for use with systems and methods disclosed herein is not limited to just primary forms of cancer, but also involves cancer subtypes. Some such cancer subtypes are listed above but also include breast cancer subtypes such as Luminal A (hormone receptor (HR)+/human epidermal growth factor receptor (HER2)−); Luminal B (HR+/HER2+); Triple-negative or (HR−/HER2−) and HER2 positive. Other cancer subtypes include the various lung cancers listed above and prostate cancer subtypes involving changes in E26 transformation specific genes (ETS; specifically ERG, ETV1/4, and FLI1 genes) and subsets defined by mutations in FOXA1, SPOP, and IDH1 genes.

Further indication of the association of cancers with alternative splicing events is the possible association of mutation of the splicing machinery with the development of cancer. As discussed in Zhang et al. (Signal Transduction and Targeted Therapy, 6(78) (2021)), the last decades have associated somatic mutations in components of the human splicing machinery with human solid tumors, including bladder, brain, breast, cervix, colon, kidney, liver, lung, oral/head and neck, ovary, prostate, skin, stomach, and thyroid tumors, as well as hematological malignancies including myeloid leukemia (AML), myelodysplastic syndrome (MDS), chronic myelogenous leukemia, de novo AML, myelodyplastic syndrome without ringed sideroblasts (MDS w/o RS), myeloproliferative neoplasm (MPN), and refactory anemoa with ringed sideroblasts, and refractory cytopenia with multlineage dysplasia and ringed sideroblasts (RARS/RCMD). In addition to cancer, neurological disease, such as Alzheimer's Disease (AD), Parkinson's disease, Huntington's disease (HD), schizophrenia, congentical myasthenic syndrome, spinal muscular atrophy, and immunological and infectious diseases, such as celiac disease, psoriasis, systemic lupus erythematosus, asthma, inflammatory response, viral infections, cardiovascular disease, and diabetes mellitus have been connected to mis-splicing events. Most of the diseases are due to either genetic mutation falling within the canonical RNA splicing sites, which directly influences mRNA maturation, or alterations in the expression level of spliceosomal/splicing regulatory factors that contribute to the splicing of pre-mRNA.

In the case of cancer, splicing errors can impact the transformation of normal cells into cancer cells because of alterations in cellular proliferation, escape from cell death, growth inhibition, induction of angiogenesis, invasion and metastasis, energy metabolism, and immune escape. In particular, altered protein production can influence proliferation and apoptosis, invasion and metastasis, and angiogenesis and metabolism. These changes in cell function can cause or promote cancerous growth. At present, there are small molecules and splice-switching antisense oligonucleotides (SSOs) that have been validated for targeting alternative splicing in the treatment of cancer. For example, Bonnal et al. Nat. Rev. Drug Discov., 11:847-859 (2012) provides discussion about the use of the spliceosome as a target for novel antitumor drugs. A common target for small molecules is the splicing of SF3B1, a protein component of the spliceosome. Some small molecules that are currently being tested in this capacity include spliceostatin A, pladienolide-B, GEX1A, and E1707. A further small molecule with promise in this area is Amiloride, which is shown to change alternative splicing of key cancer-associated molecules such as Bcl-x, HIPK3, and RON/MISTR1. A still further small molecule is H3B-8800, which is now in a phase 1 clinical trial (NCT02841540) to target relapsed/refractory myeloid neoplasms (MDS, CMML, and AML) that carry splicing factor mutations (see, Zhang et al. Signal Transduction and Targeted Therapy, 6(78) (2021)). It is anticipated that the systems and methods disclosed herein could detect and connect such mutations in individual patient samples to these possible treatment methods.

One possible further target would be the mis-spliced RNA transcripts themselves with SSOs, anti-sense oligonucleotides (ASO), short hairpin RNA interference/small interference RNA, clustered regularly interspaced short palindromic repeats (CRISPR)-associated (Cas) system, such as the CRISPR-Cas13a enzyme, or single-base editors (BEs, in particular cytosine-BEs (CBEs) or adenosine-BEs (ABEs). The prototype for SSO based gene therapy treatments is the now approved treatment of the neurological diseases spinal muscular atropy (SMA) and Duchenne muscular dystrophy using ASOs. Numerous clinical trials are now underway for other neurological diseases such as mytonic dystrophy, HD, amyotrophic lateral sclerosis (ALS) and AD.

However, it is anticipated that such treatment approaches will be developed in other diseases impacted by alternative splicing such as cancer. For example, the base-pairing of oligonucleotides to RNA can induce degradation of or interfere with the splicing of pre-mRNA. In order to improve the stability of synthetic oligonucleotides, replacing the ribose ring of the oligonucleotide subunits with a morpholine ring, termed morpholino, seems especially suitable for targeting splicing, as termed morpholino are refractory to RNase H activity and thus not directly degrade the pre-mRNA. Studies have shown that Bcl-x SSOs could be combined with the downstream 5′ SS of the exon 2 in prem-RNA of Bcl-x and modify Bcl-x pre-mRNA splicing. The pro-apoptotic effect on tumor cell lines demonstrates the anti-tumor activity of Bcl-x pre-mRNA spliced SSO. The decoy RNA oligonucleotides were designed and confirmed to inhibit the splicing and biological activity of RBFOX1/2, SRSF1 and PTBP1. Therefore, SSOs will be an effective way to treat tumors caused by the vital mis-spliced events during disease initiation and/or progression. It is anticipated that the systems and methods disclosed herein will be equally able to connect patient sample results with the possible use of these treatment methods as they are developed.

A further suggested treatment could be antibodies against tumor-specific neo-antigens caused by alternative splicing. There have been a few experimentally validated splicing-derived peptides with neo-epitopes that are recognized by T cells with evidence of immunogenicity. In a study on chronic myeloid leukemia, peptides derived from alternatively spliced out-of-frame BCR/ABL fusing transcripts were able to stimulate a peptide-specific cytotoxic T lymphocyte response, evidenced by the detection of out-of-frame peptide-specific IFN7+CD8+ T cells in patients and the killing of peptide-pulsed target cells in vitro by these cytotoxic T lymphocytes. Another recent study on B-cell lineage marker CD20 showed that its alternative splicing isoform with a 168-nucleotide spliced out in exons 3-7 was only present in several patient-derived B lymphoma cell lines but not normal cells, and could generate a CD20-derived peptide with HLA-DR1 binding epitopes and vaccination, thus eliciting epitope-specific CD4+ and CD8+ responses in transgenic mice. Any or all of these immune-based treatment methods could be suggested treatments based on the findings of the systems and methods disclosed herein.

A consideration for both the systems and methods disclosed herein and the likely success for neo-antigen targeted therapy is the issue of tumor clonal heterogeneity. It is anticipated that the present method can function effectively for connection of a particular patient sample with a particular treatment method where the tumor has as low as about 30% to about 20% tumor purity. Thus, for select diseases or to screen for the applicability of specific therapies such as those involving neo-antigen immunological based targeting, the systems and methods disclosed herein can include a pre-screen of the provided patient sample for tumor purity to evaluate the applicability of the systems and methods disclosed herein to the patient sample at issue. A specimen having low tumor purity may be subjected to microdissection in an attempt to isolate the cancer cells and generate a new specimen having a higher tumor purity, on which the systems and methods may be used. Various methods of measuring tumor purity are known in the art. Tumor purity is the proportion of cancer cells in the admixture. Until recently, it was estimated by a pathologist, primarily by visual or image analysis of tumor cells. With the advancement of genomic technologies, many new computational methods have arisen to infer tumor purity. These methods make estimates using different types of genomic information, such as gene expression, somatic copy-number variation, somatic mutations and DNA methylation (see, Aran et al., Nature Comm. 6:8971 (2015)).

Further uses of this method is when the disease is a thalassemia (see, e.g. Cao and Galanello, Genet. in Med., 12:61-76 (2010)), familial dysautonomia (see, e.g., Slaugenhaupt et al., Am. J. Hum. Genet., 68(3): 598-605); spinal muscular atrophy (see, e.g., Singh and Singh, RNA Biol., 8(4):600-6 (2011)), amyotrophic lateral sclerosis (see, e.g., Jin et al., Neoplasia, 22(9):447-57 (2020)), or Parkinson's disease (see, e.g. Fu et al., Cell Transplant. 22(4): 653-61 (2013)). The splice profiles of the systems and methods disclosed herein are anticipated to be useful for any disease which has been associated or is suspected to be associated with alternative splicing, particularly when such alternative splicing provides supportive data for diagnostic, prognosis, treatment methods, or other clinically or research-related aspects of patient care.

For embodiments of the systems and methods disclosed herein, the specific computational format for the matching between a patient sample alternative splicing results, the disease at issue, and potential treatment methods is in the form of a manually curated knowledge database. Such a database will record the particular splicing variant, including the gene involved with the disease state, applicable therapies, and ultimately, with the outcome of such therapies. Each newly identified splice variant is recorded into this database as one or more local events. The local nature of the events makes it difficult to compare to the whole sequences of constitutive splicing molecules, for example, non-principal isoform sequences reported, documented, and/or stored in databases. It is this aspect of the produced data that results in the need for the knowledge database to be manually curated. This curated database will provide basis for future assignment of similar splicing variants to the possible suggested use of therapies, particularly those where there have been positive outcomes. Alternative approaches to a fully manually curated knowledge database is an artificial intelligence driven curated database. Databases that associate particular patient outcomes and other patient characteristics such as gene expression values to particular therapies and their outcome are known in the art, see for example U.S. Pat. No. 10,600,503 (Systems medicine platform for personalized oncology); U.S. Patent Publ. No. 20060136143 (Personalized genetic-based analysis of medical conditions); and U.S. Patent Publ. No. 20080082522 (Computational systems for biomedical data).

### D. Systems

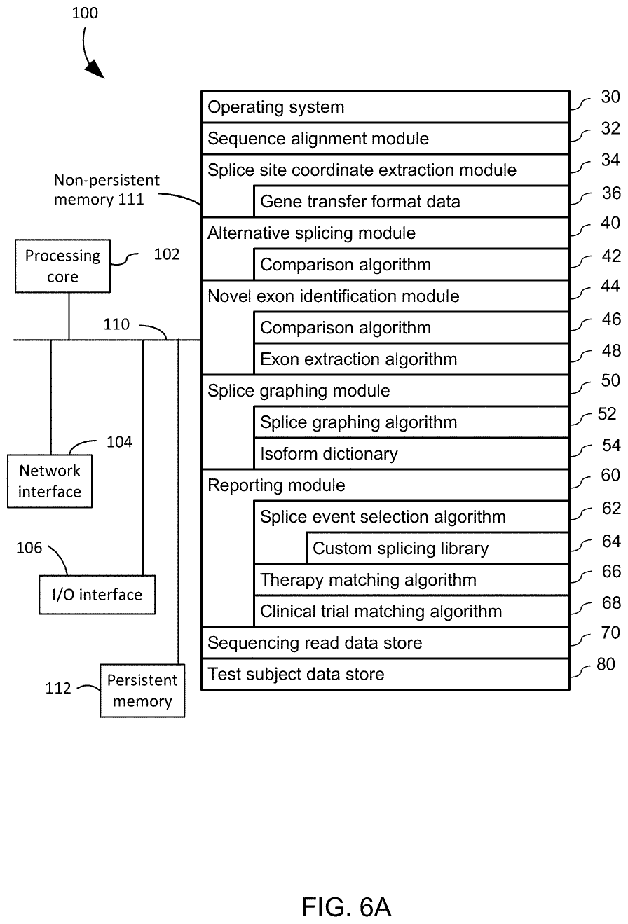

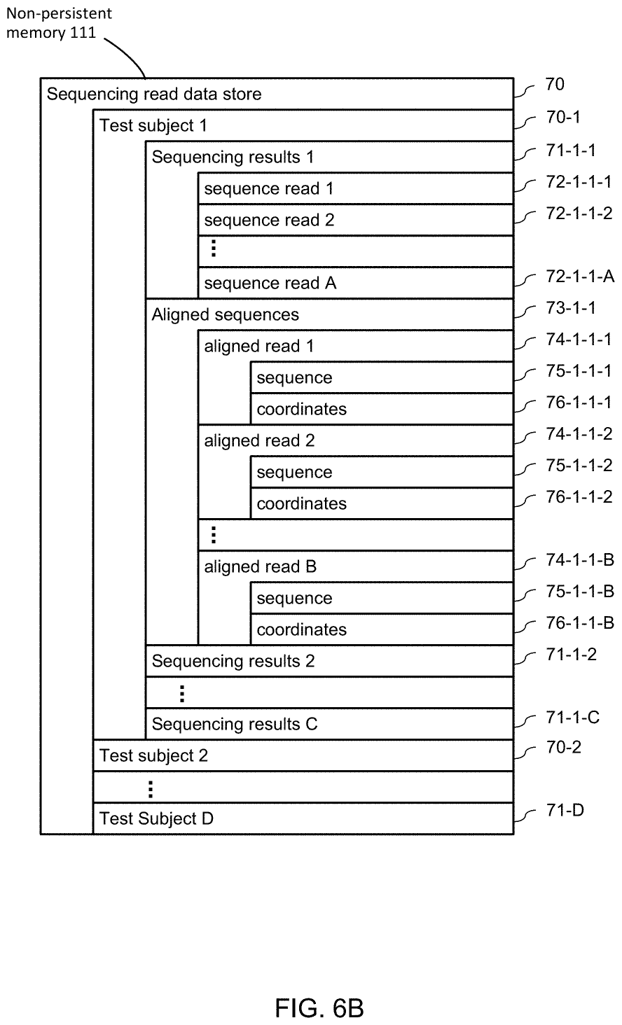

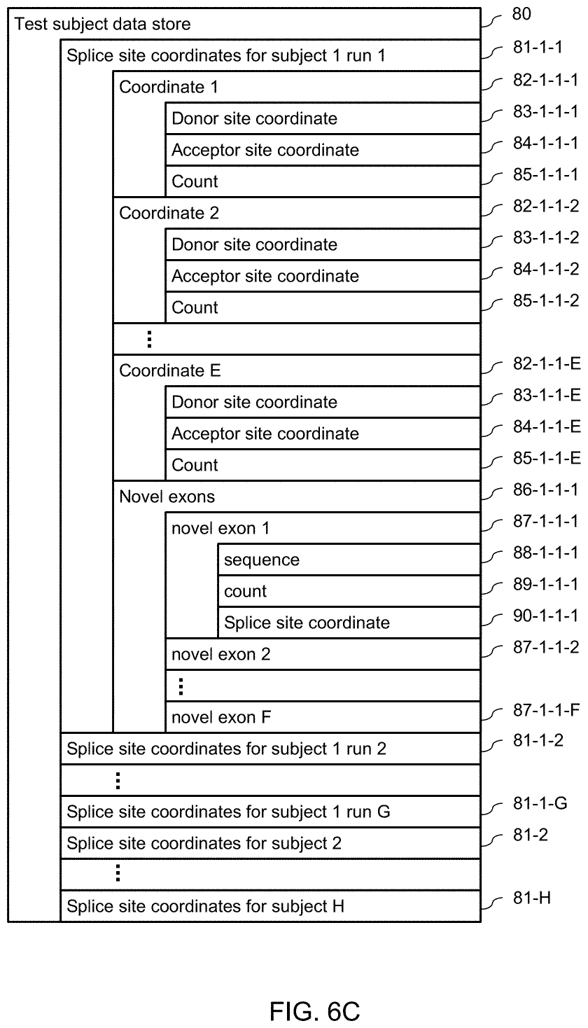

FIGS. 6A-6C collectively show a block diagram illustrating a system 100 for mapping splicing events in a test subject, in accordance with some implementations. The device 100 in some implementations includes one or more central processing units (CPU(s)) 102 (also referred to as processors), one or more network interfaces 104, a user interface 106, a non-persistent memory 111, a persistent memory 112, and one or more communication buses 110 for interconnecting these components. The one or more communication buses 110 optionally include circuitry (sometimes called a chipset) that interconnects and controls communications between system components. The non-persistent memory 111 typically includes high-speed random access memory, such as DRAM, SRAM, DDR RAM, ROM, EEPROM, flash memory, whereas the persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 112 optionally includes one or more storage devices remotely located from the CPU(s) 102. The persistent memory 112, and the non-volatile memory device(s) within the non-persistent memory 112, comprises non-transitory computer readable storage medium. In some implementations, the non-persistent memory 111 or alternatively the non-transitory computer readable storage medium stores the following programs, modules and data structures, or a subset thereof, sometimes in conjunction with the persistent memory 112:
an optional operating system 30, which includes procedures for handling various basic system services and for performing hardware dependent tasks;
an optional network communication module (or instructions) for connecting the system 100 with other devices, or a communication network;
an optional sequence alignment module 32 for aligning sequence reads (e.g., sequence reads 72), e.g., generated from an mRNA sample, to a reference construct for the species of the subject, e.g., a reference genome, exome, transcriptome, or other partial genomic construct;
an optional splice site coordinate extraction module 34 for extracting, e.g., directly from raw or de-duplicated unique sequence reads or from aligned sequence reads, splice site coordinates (e.g., splice site coordinates 82), including a corresponding donor splice site coordinate (e.g., donor site coordinates 83) and a corresponding acceptor splice site coordinate (e.g., acceptor site coordinates 84) for each splice site in the sequence read (e.g., sequence reads 72), optionally using gene transfer format data 36 that maps genes and/or subgenic structures (e.g., exons and introns) to a reference construct (e.g., reference genome) for the species of the subject;
an alternative splicing module 40 for mapping splicing events in sequence reads (e.g., sequence reads 72), including:
   a comparison algorithm 42 for comparing splice site coordinates (e.g., splice site coordinates 82) identified in the sequence reads to reference splice site coordinates for one or more known mRNA constructs for a respective gene to identify constitutional and alternative splice events; and
an optional novel exon identification module 44 for identifying novel exons (e.g., novel exons 87) based on splice site coordinates (e.g., splice site coordinates 82) that do not correspond to constitutive or known alternative splicing patterns for a respective gene, including:
   a comparison algorithm 46 for comparing a donor splice site coordinate (e.g., donor site coordinates 83) and/or an acceptor splice site coordinate (e.g., acceptor site coordinates 84) not present in one or more known mRNA constructs for a respective gene to a genomic construct for the gene; and
   an exon extraction algorithm 48 for identifying novel exons based on identification of a predicted donor splice site and/or predicted acceptor splice site located near the novel donor splice site and/or novel acceptor splice site in a genomic construct for the respective gene;
an optional splice graphing module 50 for aggregating and/or annotating novel splicing events and/or novel mRNA isoforms for a respective gene based on identified novel exons, including;
   a splice graphing algorithm 52, e.g., for generating alternative splicing maps for a transcript, optionally using an isoform dictionary 54 for the respective gene that may be updated with novel mRNA isoforms identified;
an optional reporting module 60, e.g., for generating a report for a clinician or patient based on at least the alternative splicing analysis described herein, including:
   an optional splice event selection algorithm 62, e.g., for selecting identified alternative splicing events for reporting, optionally using a custom splicing library 64 containing the identity of one or more alternative splice events to be reported;
   an optional therapy matching algorithm 66 for providing therapeutic recommendations based on an identified alternative splicing pattern, (e.g., a presence of a particular alternative splicing event, an absence of a particular alternative splicing event, and/or a quantification of one or more alternative splicing events), e.g., optionally including a look-up table (LUT) associating one or more alternative splicing patterns with one or more recommended and/or eligible therapies; and
   an optional clinical trial matching algorithm 68 for identifying clinical trials a subject may be eligible for based on an identified alternative splicing platform, e.g., optionally including a LUT associating one or more alternative splicing pattern with one or more clinical trials;
an optional sequence read data store 70 for storing sequence read data for one or more test subjects (e.g., test subject 70-1) from one or more sequencing runs 71, including sequence reads 72 and/or aligned sequences 73 including sequences 75 and genomic coordinates 76, for use in the splicing analyses described herein; and
an optional test subject data store 80 for storing outputs from the splicing analyses described herein, including:
   sets of splice site coordinates 81 from a sequencing result 71, including identified splice site coordinates 82 comprising a corresponding donor splice site coordinate 83 and a corresponding acceptor splice site coordinate, and an optional count 85 for the number of unique occurrences of the splice site coordinate 82 in the sequencing results 71; and/or
   sets of novel exons 86 identified from non-constitutional splice site coordinates 82, including for each novel exon 87 a corresponding sequence 88 for the exon 87, a corresponding count 89 for the number of unique occurrences of the novel exon in the sequencing results 71, and the identity 90 of the non-constitutional splice site used to identify the novel exon 87.

In some implementations, one or more of the above identified elements are stored in one or more of the previously mentioned memory devices and correspond to a set of instructions for performing a function described above. The above identified modules, data, or programs (e.g., sets of instructions) need not be implemented as separate software programs, procedures, data sets, or modules, and thus various subsets of these modules and data may be combined or otherwise re-arranged in various implementations. In some implementations, the non-persistent memory 111 optionally stores a subset of the modules and data structures identified above. Furthermore, in some embodiments, the memory stores additional modules and data structures not described above. In some embodiments, one or more of the above identified elements is stored in a computer system, other than that of system 100, that is addressable by system 100 so that system 100 may retrieve all or a portion of such data when needed.

Although FIGS. 6A-6C depict a “system 100,” the figures are intended more as a functional description of the various features which may be present in computer systems than as a structural schematic of the implementations described herein. In practice, and as recognized by those of ordinary skill in the art, items shown separately could be combined and some items could be separated. Moreover, although FIGS. 6A-6C depict certain data and modules in non-persistent memory 111, some or all of these data and modules may be in persistent memory 112.

Some embodiments of the systems and methods disclosed herein involve systems that have been configured for the performance of steps of the present methods. Such systems can be described as comprising primarily a computational device. At a minimum, the systems will comprise at least one processor and at least one memory. The device in some implementations includes one or more processing units CPU(s) (also referred to as processors), one or more network interfaces, a user interface, for example, including a display and/or an input (for example, a mouse, touchpad, keyboard, etc.), a non-persistent memory, a persistent memory, and one or more communication buses for interconnecting these components. The one or more communication buses optionally include circuitry (sometimes called a chipset) that interconnects and controls communications between system components. The non-persistent memory typically includes high-speed random-access memory, such as DRAM, SRAM, DDR RAM, ROM, EEPROM, flash memory, whereas the persistent memory typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices.

The persistent memory optionally includes one or more storage devices remotely located from the CPU(s). The persistent memory, and the non-volatile memory device(s) within the non-persistent memory, comprise non-transitory computer readable storage medium. In some implementations, the non-persistent memory or alternatively the non-transitory computer readable storage medium stores the following programs, modules and data structures, or a subset thereof, sometimes in conjunction with the persistent memory: an operating system, which includes procedures for handling various basic system services and for performing hardware dependent tasks; a network communication module (or instructions) for connecting the system with other devices and/or a communication network; a test patient data store for storing one or more collections of features from patients (for example, subjects); a bioinformatics module for processing sequencing data and extracting features from sequencing data, for example, from liquid biopsy, solid tumor, or other sequencing assays, including next generation sequencing assays; a feature analysis module for evaluating patient features, for example, genomic alterations, compound genomic features, and clinical features; and a reporting module 1 for generating and transmitting reports that provide clinical support for personalized cancer therapy.

Although the above description depicts a “system,” this description is intended more as a functional description of the various features that may be present in computer systems than as a structural schematic of the implementations described herein. In practice, and as recognized by those of ordinary skill in the art, items shown separately could be combined and some items could be separated. The relationship between persistent and non-persistent memory described in possible association that is not intended to be limiting. For example, in various implementations, one or more of the above identified elements are stored in one or more of the previously mentioned memory devices, and correspond to a set of instructions for performing a function described above. The above identified modules, data, or programs (for example, sets of instructions) need not be implemented as separate software programs, procedures, datasets, or modules, and thus various subsets of these modules and data may be combined or otherwise re-arranged in various implementations.

In some implementations, the non-persistent memory optionally stores a subset of the modules and data structures identified above. Furthermore, in some embodiments, the memory stores additional modules and data structures not described above. In some embodiments, one or more of the above-identified elements is stored in a computer system, other than that of the system, that is addressable by the system so that the system may retrieve all or a portion of such data when needed.

One such illustrative example is the system as a single computer that includes all of the functionality for providing methods of detecting alternative splicing variants. However, while a single machine is possible, the term “system” shall also be taken to include any collection of machines that individually or jointly execute a set (or multiple sets) of instructions to perform any one or more of the methodologies discussed herein.

For example, in some embodiments, the system includes one or more computers. In some embodiments, the functionality for detecting, classifying, and documenting alternative splicing variants is spread across any number of networked computers and/or resides on each of several networked computers and/or is hosted on one or more virtual machines at a remote location accessible across the communications network. For example, different portions of the various modules and data stores can be stored and/or executed on the various instances of a processing device and/or processing server/database in the distributed diagnostic environment (for example, multiple processing devices, a processing server, and a database).

The system may operate in the capacity of a server or a client machine in client-server network environment, as a peer machine in a peer-to-peer (or distributed) network environment, or as a server or a client machine in a cloud computing infrastructure or environment. The system may be a personal computer (PC), a tablet PC, a set-top box (STB), a Personal Digital Assistant (PDA), a cellular telephone, a web appliance, a server, a network router, a switch or bridge, or any machine capable of executing a set of instructions (sequential or otherwise) that specify actions to be taken by that machine.

In another implementation, the system comprises a virtual machine that includes a module for executing instructions for performing any one or more of the methodologies disclosed herein. In computing, a virtual machine (VM) is an emulation of a computer system that is based on computer architectures and provides functionality of a physical computer. Some such implementations may involve specialized hardware, software, or a combination of hardware and software.

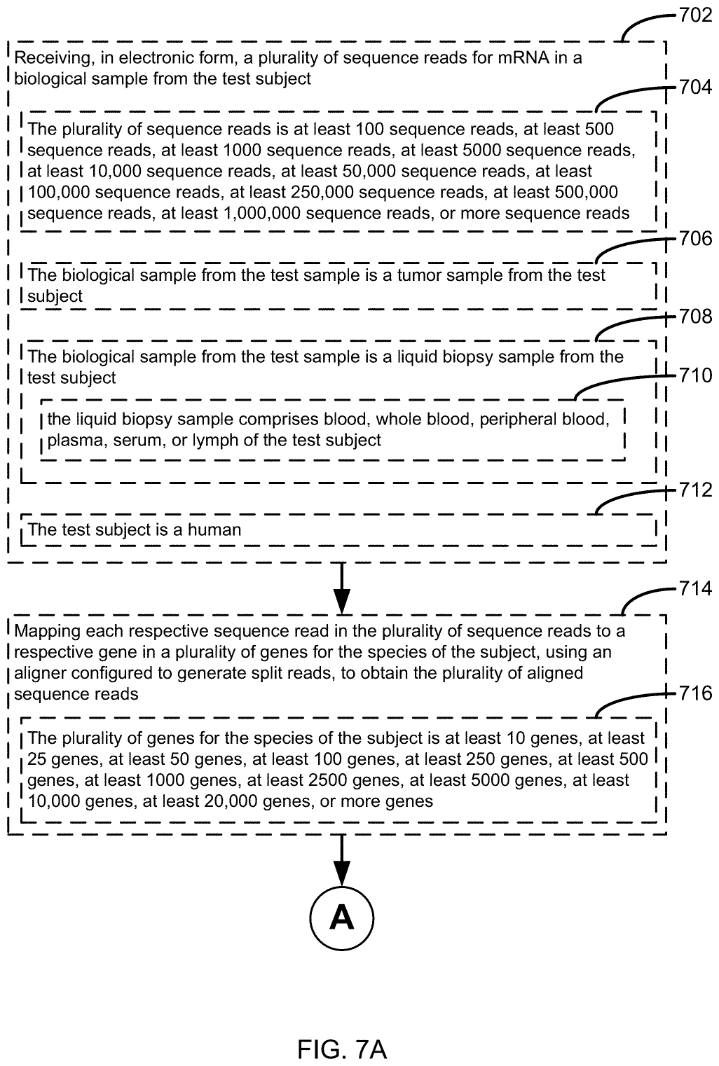

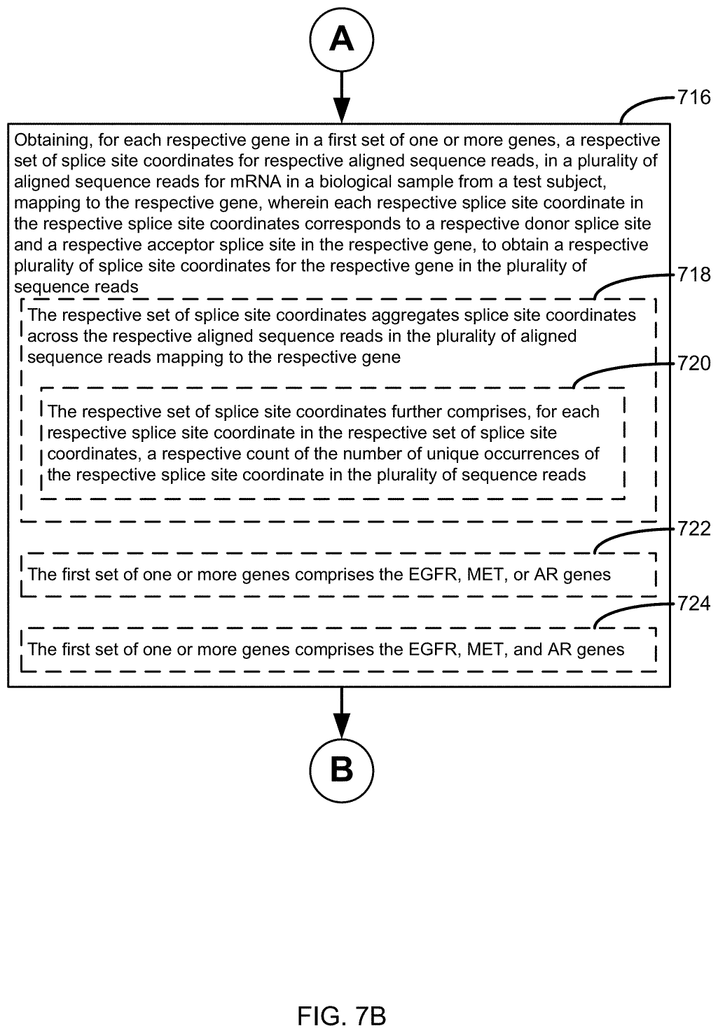

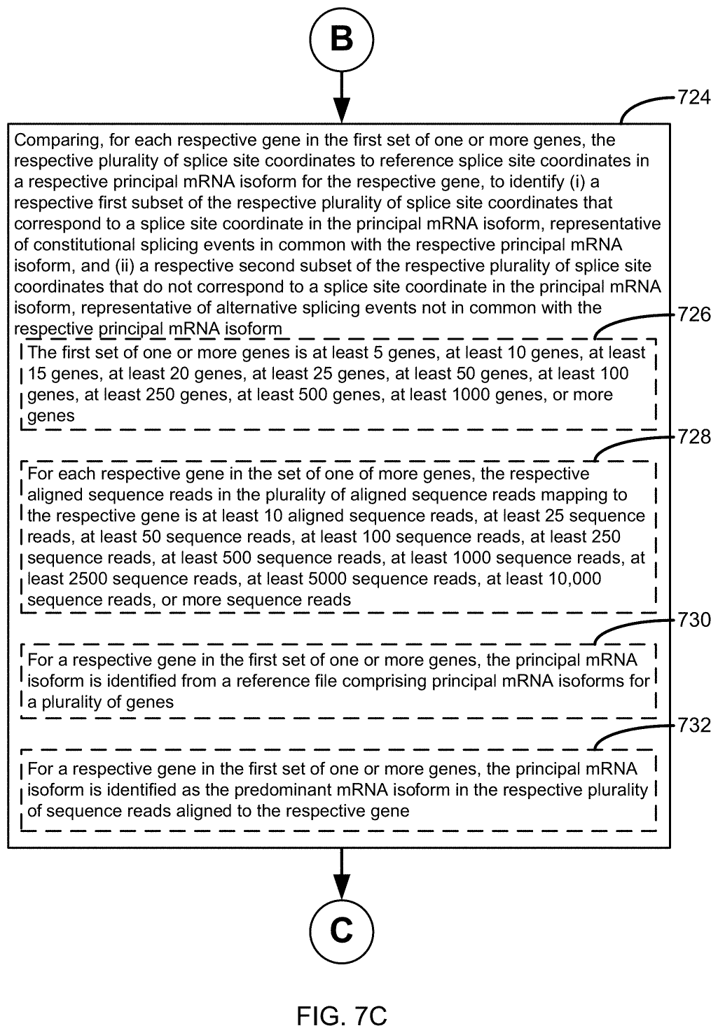

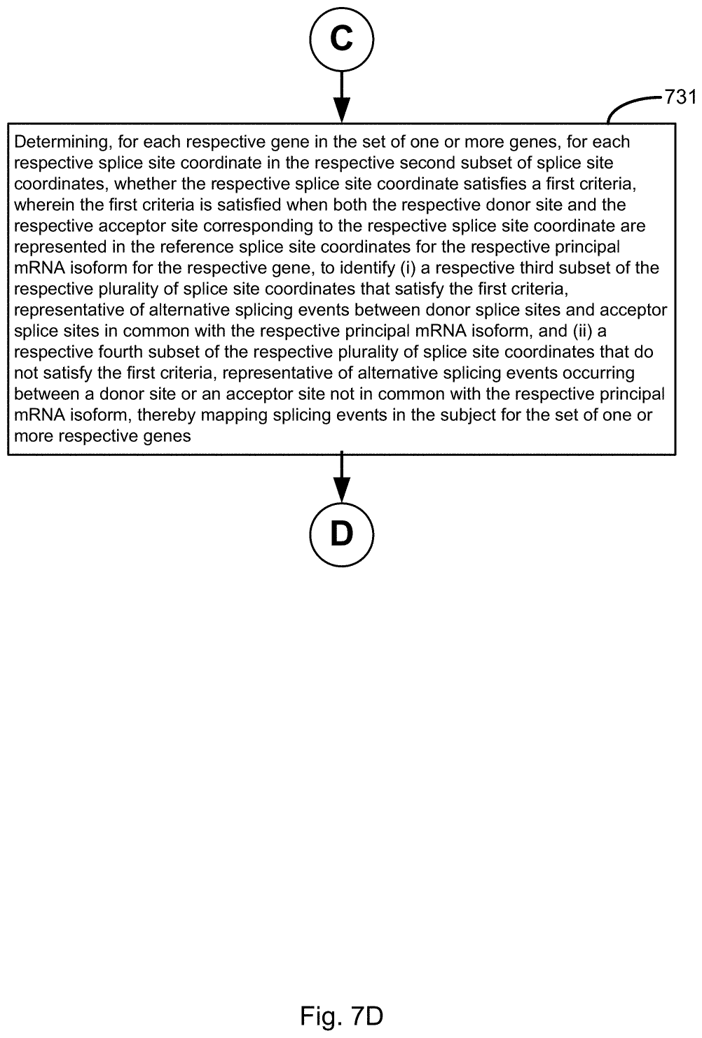

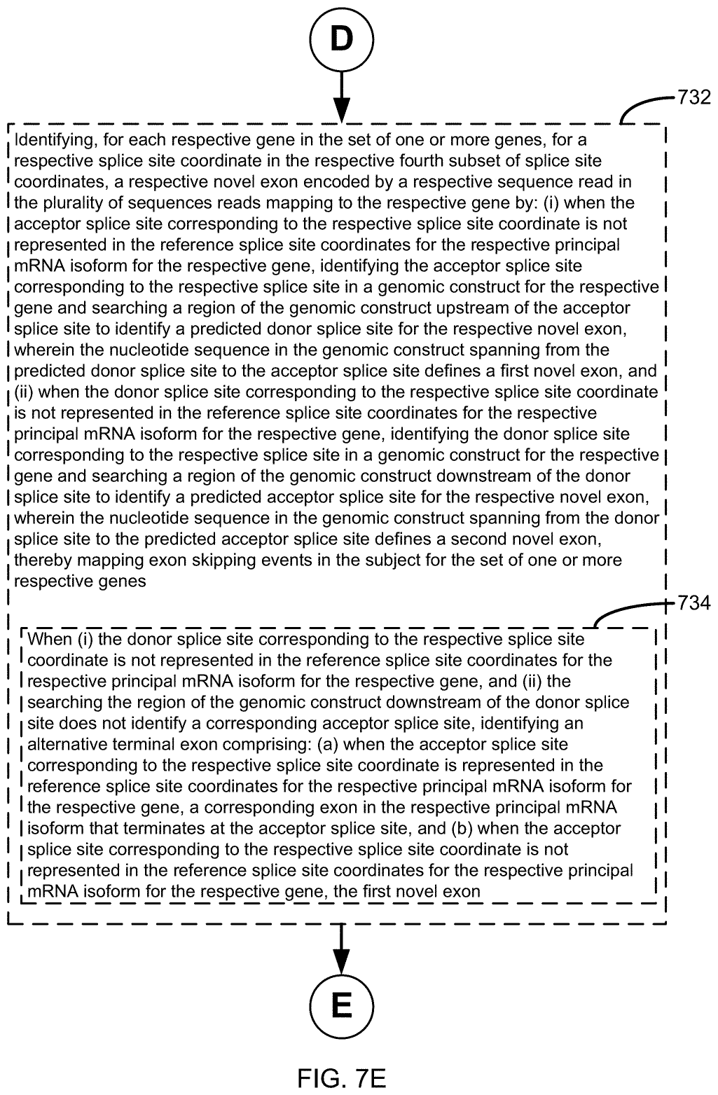

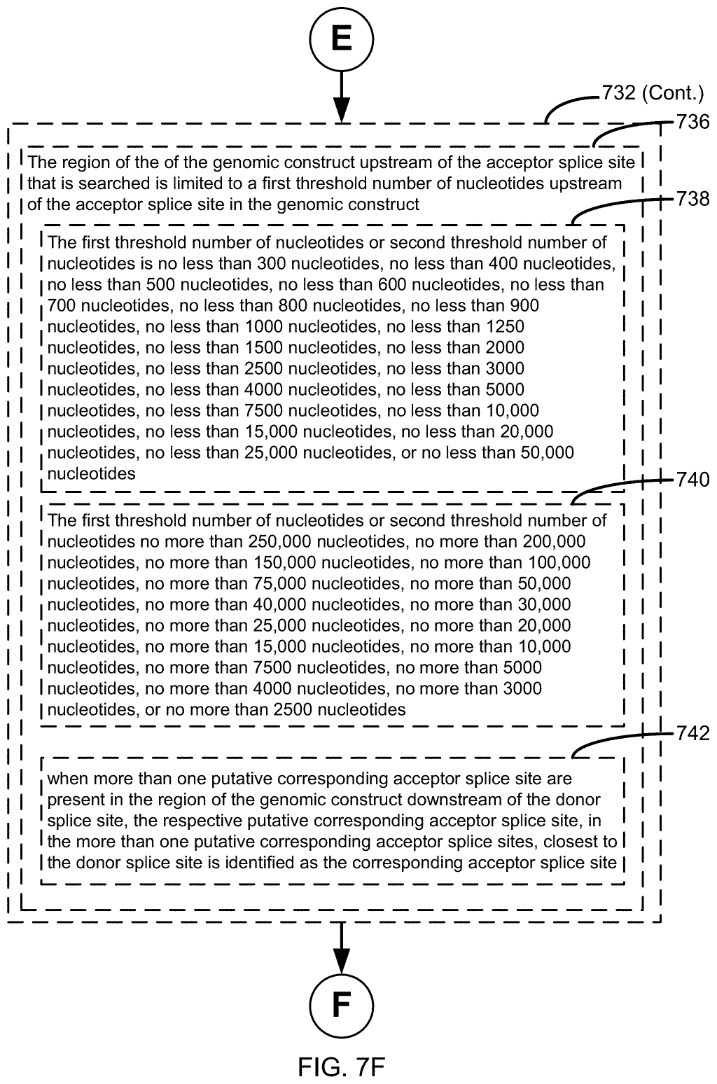

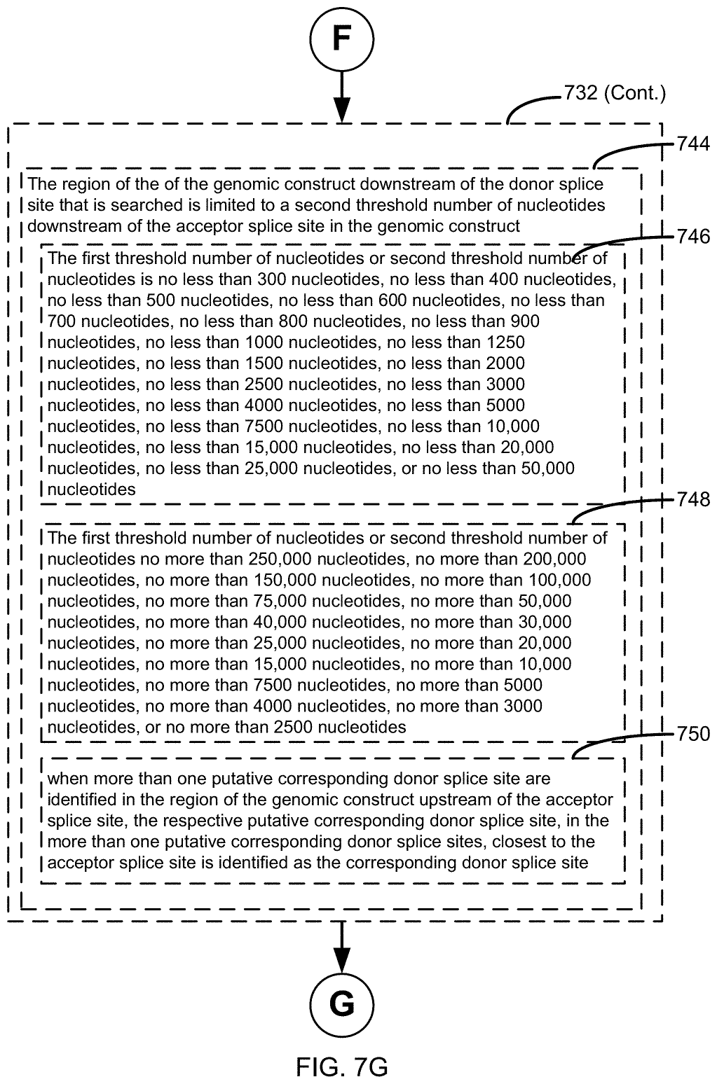

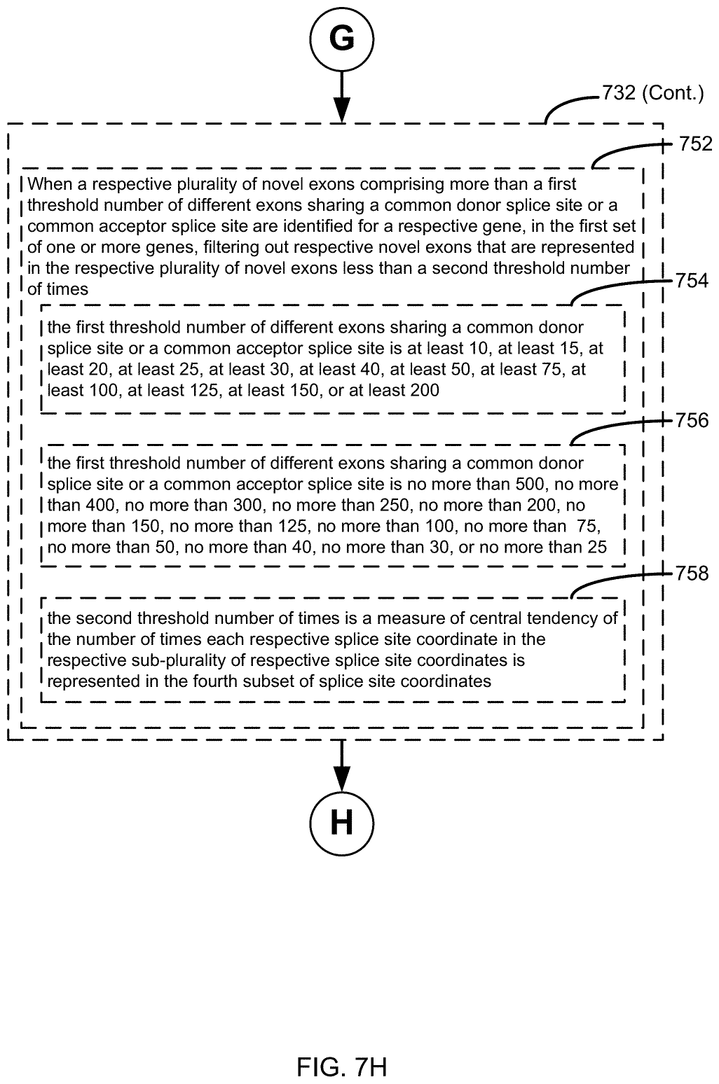

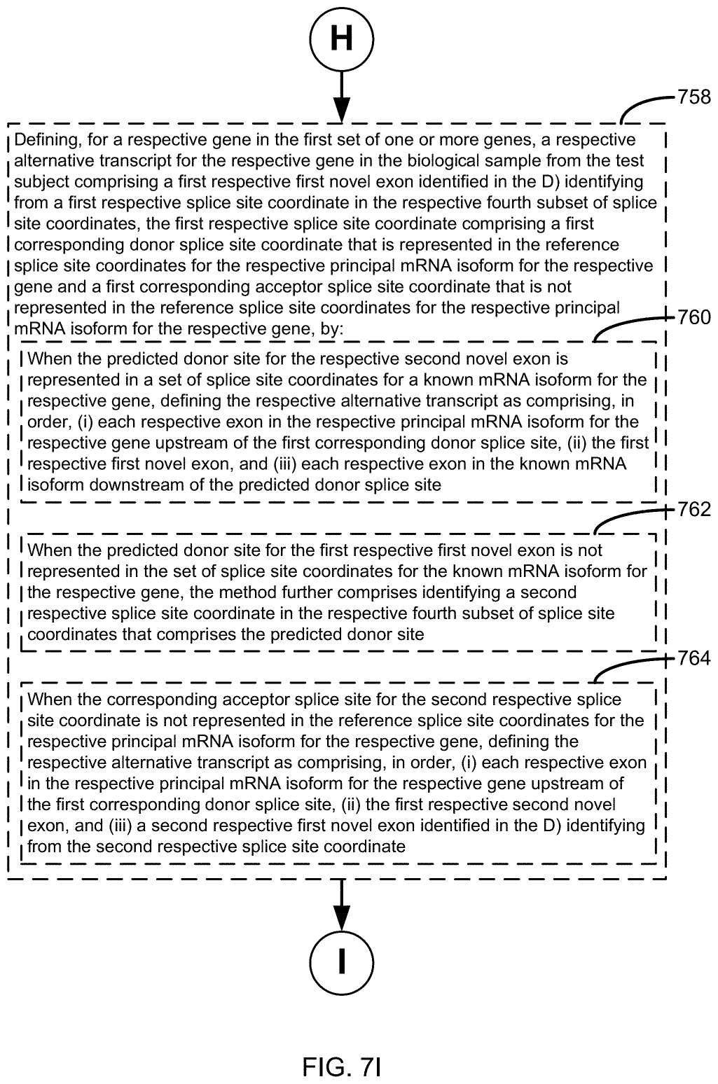

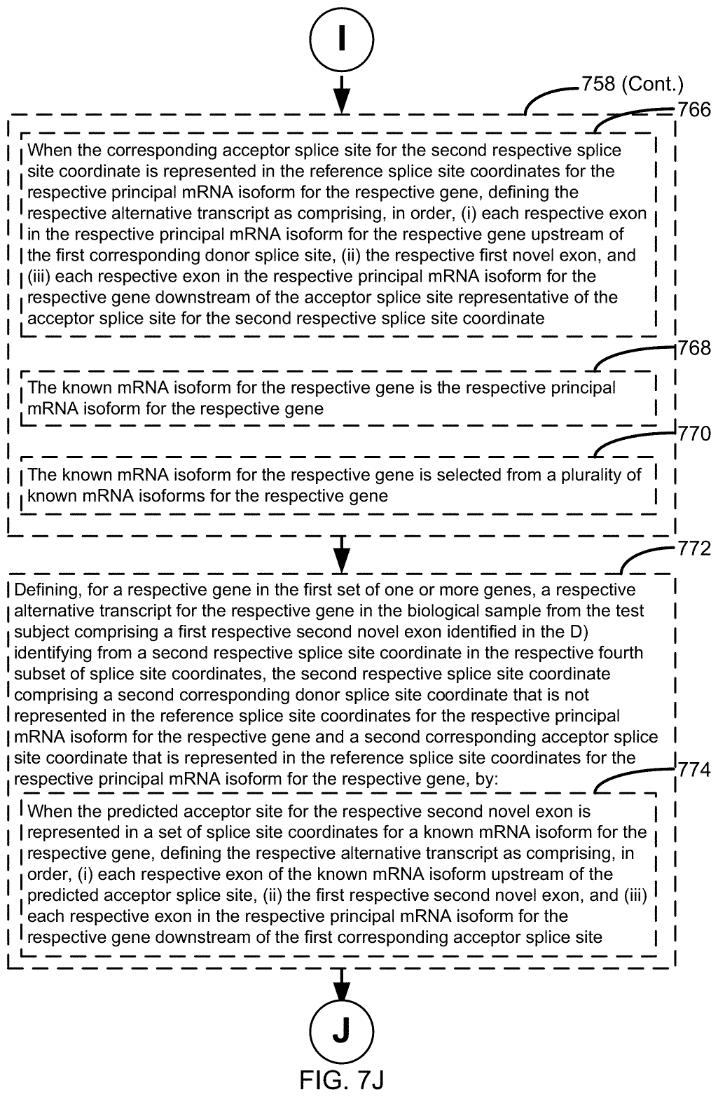

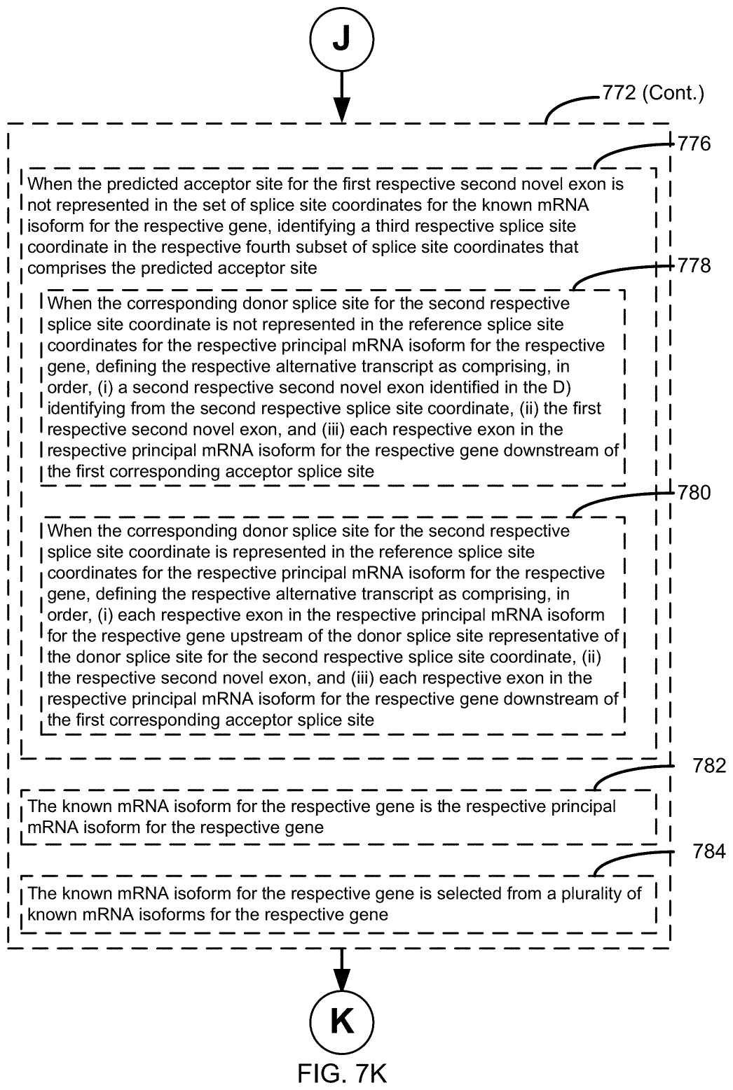

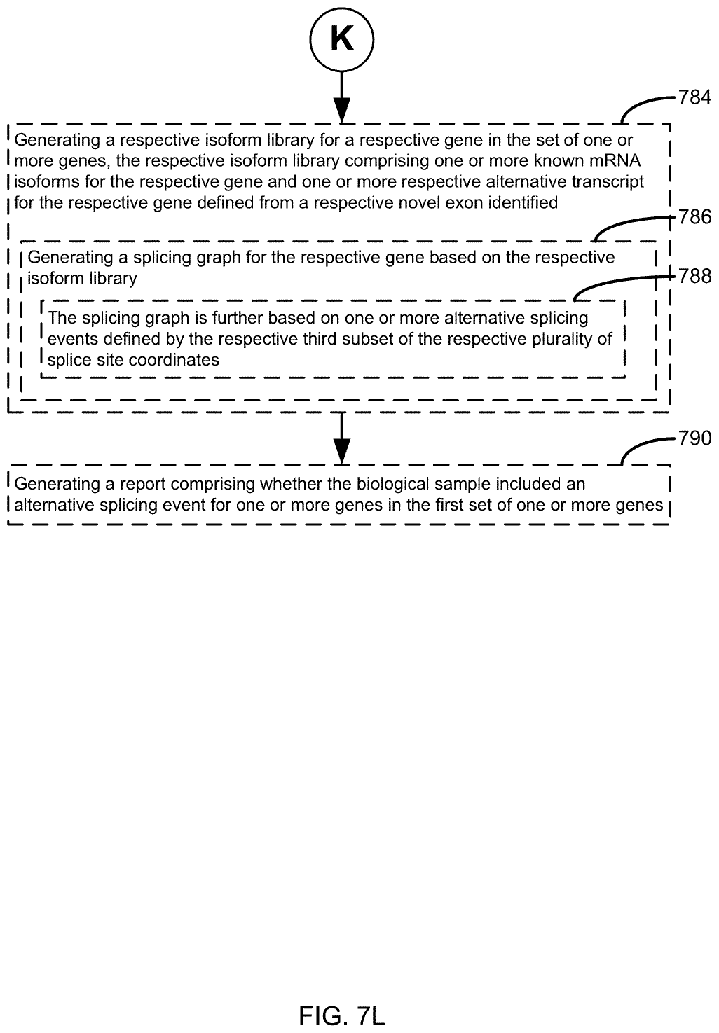

While systems in accordance with the present disclosure have been disclosed with reference to FIGS. 6A-6C, methods in accordance with the present disclosure are now detailed with reference to FIGS. 7A-7K.

In some embodiments, the disclosure provides a method 700 for mapping splicing events in a test subject. In some embodiments, such methods are preformed at a computer system (e.g., system 100) comprising at least one processor and a memory storing at least one program for execution by the at least one processor.

With reference to blocks 702-712, in some embodiments, the method includes obtaining sequence read data for mRNA from a biological sample of a subject. Referring to block 702, in some embodiments, the method includes receiving, in electronic form, a plurality of sequence reads for mRNA in a biological sample from the test subject. Referring to block 704, in some embodiments, the plurality of sequence reads is at least 100 sequence reads, at least 500 sequence reads, at least 1000 sequence reads, at least 5000 sequence reads, at least 10,000 sequence reads, at least 50,000 sequence reads, at least 100,000 sequence reads, at least 250,000 sequence reads, at least 500,000 sequence reads, at least 1,000,000 sequence reads, or more sequence reads. Referring to block 706, in some embodiments, the biological sample from the test sample is a tumor sample from the test subject. Referring to block 708, in some embodiments, the biological sample from the test sample is a liquid biopsy sample from the test subject. Referring to block 710, in some embodiments, the liquid biopsy sample includes blood, whole blood, peripheral blood, plasma, serum, or lymph of the test subject. Referring to block 712, in some embodiments, the test subject is a human.

With reference to blocks 714-722, in some embodiments the method includes aligning sequences reads from the sequence read data to a reference construct for the species of the subject, e.g., a reference genome, a reference exome, a reference transcriptome, or a partial reference construct thereof. Similarly, in some embodiments, the method includes identifying splice site coordinates, including, for each respective splice site coordinate, a coordinate for a donor splice site and a coordinate for an acceptor splice site that have been spliced together in the sequencing data. However, in some embodiments, method 700 begins by accessing previously aligned sequence data and/or previously extracted splice site coordinates from the sequence data, rather than performing the alignment and/or splice site coordinate identification.

Referring to block 714, in some embodiments, the method includes mapping each respective sequence read in the plurality of sequence reads to a respective gene in a plurality of genes for the species of the subject, using an aligner, e.g., an aligner configured to generate split reads, to obtain the plurality of aligned sequence reads. Referring to block 716, in some embodiments, the plurality of genes for the species of the subject is at least 10 genes, at least 25 genes, at least 50 genes, at least 100 genes, at least 250 genes, at least 500 genes, at least 1000 genes, at least 2500 genes, at least 5000 genes, at least 10,000 genes, at least 20,000 genes, or more genes. Referring to block 716, in some embodiments, the method includes generating, for each respective gene in a first set of one or more genes, a respective set of splice site coordinates for respective aligned sequence reads, in a plurality of aligned sequence reads for mRNA in a biological sample from a test subject, mapping to the respective gene, where each respective splice site coordinate in the respective splice site coordinates corresponds to a respective donor splice site and a respective acceptor splice site in the respective gene, to obtain a respective plurality of splice site coordinates for the respective gene in the plurality of sequence reads. Referring to block 718, in some embodiments, the respective set of splice site coordinates aggregates splice site coordinates across the respective aligned sequence reads in the plurality of aligned sequence reads mapping to the respective gene.

Referring to block 720, in some embodiments, the respective set of splice site coordinates further includes, for each respective splice site coordinate in the respective set of splice site coordinates, a respective count of the number of unique occurrences of the respective splice site coordinate in the plurality of sequence reads. Referring to block 722, in some embodiments, the first set of one or more genes includes the EGFR, MET, or AR genes. Referring to block 724, in some embodiments, the first set of one or more genes includes the EGFR, MET, and AR genes.

With reference to blocks 724-730, in some embodiments the method includes characterizing splice site coordinates extracted from the sequencing data, e.g., as corresponding to a constitutive splicing event (e.g., occurring during splicing of a principal mRNA isoform for a respective gene), an alternative splicing event between known and/or constitutive exons present in a known mRNA isoform (e.g., a principal mRNA isoform for a respective gene), or as a novel splicing event, e.g., involving a previously unidentified and/or non-constitutive exon present in a known mRNA isoform (e.g., a principal mRNA isoform for a respective gene).

Referring to block 724, in some embodiments, the method includes comparing, for each respective gene in the first set of one or more genes, the respective plurality of splice site coordinates to reference splice site coordinates in a respective principal mRNA isoform for the respective gene, to identify (i) a respective first subset of the respective plurality of splice site coordinates that correspond to a splice site coordinate in the principal mRNA isoform, representative of constitutional splicing events in common with the respective principal mRNA isoform, and (ii) a respective second subset of the respective plurality of splice site coordinates that do not correspond to a splice site coordinate in the principal mRNA isoform, representative of alternative splicing events not in common with the respective principal mRNA isoform.

Referring to block 726, in some embodiments, the first set of one or more genes is at least 5 genes, at least 10 genes, at least 15 genes, at least 20 genes, at least 25 genes, at least 50 genes, at least 100 genes, at least 250 genes, at least 500 genes, at least 1000 genes, or more genes. Referring to block 728, in some embodiments, for each respective gene in the set of one of more genes, the respective aligned sequence reads in the plurality of aligned sequence reads mapping to the respective gene is at least 10 aligned sequence reads, at least 25 sequence reads, at least 50 sequence reads, at least 100 sequence reads, at least 250 sequence reads, at least 500 sequence reads, at least 1000 sequence reads, at least 2500 sequence reads, at least 5000 sequence reads, at least 10,000 sequence reads, or more sequence reads.

Referring to block 730, in some embodiments, for a respective gene in the first set of one or more genes, the principal mRNA isoform is identified from a reference file including principal mRNA isoforms for a plurality of genes. Referring to block 732, in some embodiments, for a respective gene in the first set of one or more genes, the principal mRNA isoform is identified as the predominant mRNA isoform in the respective plurality of sequence reads aligned to the respective gene.

With reference to block 731, in some embodiments, the method includes determining whether splice site coordinates extracted from the sequencing data correspond to splicing events in a reference transcript for a respective gene, e.g., a principal mRNA isoform for the gene. In some embodiments, this is accomplished by comparing the identified splice site coordinates with splice site coordinates for the reference transcript and categorizing a splice site coordinate as either corresponding to a constitutional splicing event, when the splice site coordinate matches a splice site coordinate in the reference transcript, or as corresponding to an alternative splicing event, when the splice site coordinate does not match a splice site coordinate in the reference transcript.

Referring to block 731, in some embodiments the method includes determining for each respective gene in the set of one or more genes, for each respective splice site coordinate in the respective second subset of splice site coordinates, whether the respective splice site coordinate satisfies a first criteria, wherein the first criteria is satisfied when both the respective donor site and the respective acceptor site corresponding to the respective splice site coordinate are represented in the reference splice site coordinates for the respective principal mRNA isoform for the respective gene, to identify (i) a respective third subset of the respective plurality of splice site coordinates that satisfy the first criteria, representative of alternative splicing events between donor splice sites and acceptor splice sites in common with the respective principal mRNA isoform, and (ii) a respective fourth subset of the respective plurality of splice site coordinates that do not satisfy the first criteria, representative of alternative splicing events occurring between a donor site or an acceptor site not in common with the respective principal mRNA isoform, thereby mapping splicing events in the subject for the set of one or more respective genes.

With reference to blocks 732-750, in some embodiments the method includes identifying novel exons based on non-constitutional splice site coordinates extracted from the mRNA sequencing data. In some embodiments, a novel exon is one in which one or both splice sites (e.g., a corresponding acceptor splice site defining a 5′ end of the exon and a corresponding donor splice site defining a 3′ end of the exon) are not present in a reference principal transcript and/or a known mRNA isoform for a respective gene. In some embodiments, the novel exons are detected by combining splice junction information with some heuristics. The process of novel exon detection can be summarized in the following steps: select novel splice junctions, defined as splice junctions connecting a splice site in the reference transcript to a splice site not in the reference transcript, or connecting two splice sites that are not in the reference transcript. Match novel acceptor sites to novel or known donor sites within a certain genomic range to build the novel exon. Similarly, match novel donor sites to novel or known acceptor sites within a certain genomic range to build the novel exon. In some embodiments, if a novel splice site cannot be match to no other splice site, it is identified as the splice site of a terminal exon. In some embodiments, when multiple acceptors are within acceptable range of the same donor, or when multiple donors are within acceptable range of the same acceptor, shorter exons are prioritized. In some embodiments, if a longer exon is within acceptable distance but there is an intervening annotated splice junction, the longer exon is filtered out. In other embodiments, a longer exon is not filtered out in favor of a shorter one, e.g., when the longer exon uses one or more previously characterized acceptor splice site or donor splice site that the shorter exon does not.

Referring to block 732, in some embodiments, the method includes identifying, for each respective gene in the set of one or more genes, for a respective splice site coordinate in the respective fourth subset of splice site coordinates, a respective novel exon encoded by a respective sequence read in the plurality of sequences reads mapping to the respective gene by: (i) when the acceptor splice site corresponding to the respective splice site coordinate is not represented in the reference splice site coordinates for the respective principal mRNA isoform for the respective gene, identifying the acceptor splice site corresponding to the respective splice site in a genomic construct for the respective gene and searching a region of the genomic construct upstream of the acceptor splice site to identify a predicted donor splice site for the respective novel exon, where the nucleotide sequence in the genomic construct spanning from the predicted donor splice site to the acceptor splice site defines a first novel exon, and (ii) when the donor splice site corresponding to the respective splice site coordinate is not represented in the reference splice site coordinates for the respective principal mRNA isoform for the respective gene, identifying the donor splice site corresponding to the respective splice site in a genomic construct for the respective gene and searching a region of the genomic construct downstream of the donor splice site to identify a predicted acceptor splice site for the respective novel exon, where the nucleotide sequence in the genomic construct spanning from the donor splice site to the predicted acceptor splice site defines a second novel exon, thereby mapping exon skipping events in the subject for the set of one or more respective genes.

Referring to block 734, in some embodiments, when (i) the donor splice site corresponding to the respective splice site coordinate is not represented in the reference splice site coordinates for the respective principal mRNA isoform for the respective gene, and (ii) the searching the region of the genomic construct downstream of the donor splice site does not identify a corresponding acceptor splice site, identifying an alternative terminal exon including: (a) when the acceptor splice site corresponding to the respective splice site coordinate is represented in the reference splice site coordinates for the respective principal mRNA isoform for the respective gene, a corresponding exon in the respective principal mRNA isoform that terminates at the acceptor splice site, and (b) when the acceptor splice site corresponding to the respective splice site coordinate is not represented in the reference splice site coordinates for the respective principal mRNA isoform for the respective gene, the first novel exon.

Referring to block 736, in some embodiments, the region of the of the genomic construct upstream of the acceptor splice site that is searched is limited to a first threshold number of nucleotides upstream of the acceptor splice site in the genomic construct. Referring to block 738, in some embodiments, the first threshold number of nucleotides or second threshold number of nucleotides is no less than 300 nucleotides, no less than 400 nucleotides, no less than 500 nucleotides, no less than 600 nucleotides, no less than 700 nucleotides, no less than 800 nucleotides, no less than 900 nucleotides, no less than 1000 nucleotides, no less than 1250 nucleotides, no less than 1500 nucleotides, no less than 2000 nucleotides, no less than 2500 nucleotides, no less than 3000 nucleotides, no less than 4000 nucleotides, no less than 5000 nucleotides, no less than 7500 nucleotides, no less than 10,000 nucleotides, no less than 15,000 nucleotides, no less than 20,000 nucleotides, no less than 25,000 nucleotides, or no less than 50,000 nucleotides. Referring to block 740, in some embodiments, the first threshold number of nucleotides or second threshold number of nucleotides no more than 250,000 nucleotides, no more than 200,000 nucleotides, no more than 150,000 nucleotides, no more than 100,000 nucleotides, no more than 75,000 nucleotides, no more than 50,000 nucleotides, no more than 40,000 nucleotides, no more than 30,000 nucleotides, no more than 25,000 nucleotides, no more than 20,000 nucleotides, no more than 15,000 nucleotides, no more than 10,000 nucleotides, no more than 7500 nucleotides, no more than 5000 nucleotides, no more than 4000 nucleotides, no more than 3000 nucleotides, or no more than 2500 nucleotides.

Referring to block 742, in some embodiments, when more than one putative corresponding acceptor splice site are present in the region of the genomic construct downstream of the donor splice site, the respective putative corresponding acceptor splice site, in the more than one putative corresponding acceptor splice sites, closest to the donor splice site is identified as the corresponding acceptor splice site.

Referring to block 744, in some embodiments, the region of the of the genomic construct downstream of the donor splice site that is searched is limited to a second threshold number of nucleotides downstream of the acceptor splice site in the genomic construct. Referring to block 746, in some embodiments, the first threshold number of nucleotides or second threshold number of nucleotides is no less than 300 nucleotides, no less than 400 nucleotides, no less than 500 nucleotides, no less than 600 nucleotides, no less than 700 nucleotides, no less than 800 nucleotides, no less than 900 nucleotides, no less than 1000 nucleotides, no less than 1250 nucleotides, no less than 1500 nucleotides, no less than 2000 nucleotides, no less than 2500 nucleotides, no less than 3000 nucleotides, no less than 4000 nucleotides, no less than 5000 nucleotides, no less than 7500 nucleotides, no less than 10,000 nucleotides, no less than 15,000 nucleotides, no less than 20,000 nucleotides, no less than 25,000 nucleotides, or no less than 50,000 nucleotides. Referring to block 748, in some embodiments, the first threshold number of nucleotides or second threshold number of nucleotides no more than 250,000 nucleotides, no more than 200,000 nucleotides, no more than 150,000 nucleotides, no more than 100,000 nucleotides, no more than 75,000 nucleotides, no more than 50,000 nucleotides, no more than 40,000 nucleotides, no more than 30,000 nucleotides, no more than 25,000 nucleotides, no more than 20,000 nucleotides, no more than 15,000 nucleotides, no more than 10,000 nucleotides, no more than 7500 nucleotides, no more than 5000 nucleotides, no more than 4000 nucleotides, no more than 3000 nucleotides, or no more than 2500 nucleotides.

Referring to block 750, in some embodiments, when more than one putative corresponding donor splice site are identified in the region of the genomic construct upstream of the acceptor splice site, the respective putative corresponding donor splice site, in the more than one putative corresponding donor splice sites, closest to the acceptor splice site is identified as the corresponding donor splice site

With reference to blocks 752-758, the method includes filtering out novel exons with overlapping splice sites, e.g., where an exon has the same acceptor site, but multiple donor sites, or vice versa. In some embodiments, one combination of splice sites is the most predominant one, in terms of read counts. However, in some embodiments, exons representing many combinations of acceptor sites and donor sites are retained to detect low-abundance isoforms, without significantly increasing the computational complexity of the method. It has been observed that in a minor but not infrequent proportion of samples, one or more genes have many alternative splicing events, and keeping all combinations of novel exons significantly expands the required computations. To address this, in some embodiments, a filter is applied to splice sites that are shared by more than a threshold number of exon combinations (e.g., at least 50 combinations). In some such embodiments, only exon combinations that are supported by at least a threshold number of reads are maintained. In some embodiments, the threshold number is the median cumulative number of reads for that splice site. In other words, for a given splice with many combinations, the number of reads supporting each combination is sorted, and the number of reads that split the cumulative sum in half is identified and used as threshold to select only the most abundant combinations of splice sites.

Referring to block 752, in some embodiments, when a respective plurality of novel exons including more than a first threshold number of different exons sharing a common donor splice site or a common acceptor splice site are identified for a respective gene, in the first set of one or more genes, filtering out respective novel exons that are represented in the respective plurality of novel exons less than a second threshold number of times.

Referring to block 754, in some embodiments, the first threshold number of different exons sharing a common donor splice site or a common acceptor splice site is at least 10, at least 15, at least 20, at least 25, at least 30, at least 40, at least 50, at least 75, at least 100, at least 125, at least 150, or at least 200. Referring to block 756, in some embodiments, the first threshold number of different exons sharing a common donor splice site or a common acceptor splice site is no more than 500, no more than 400, no more than 300, no more than 250, no more than 200, no more than 150, no more than 125, no more than 100, no more than 75, no more than 50, no more than 40, no more than 30, or no more than 25. Referring to block 758, in some embodiments, the second threshold number of times is a measure of central tendency of the number of times each respective splice site coordinate in the respective sub-plurality of respective splice site coordinates is represented in the fourth subset of splice site coordinates.

Referring to block 758, in some embodiments, the method includes defining, for a respective gene in the first set of one or more genes, a respective alternative transcript for the respective gene in the biological sample from the test subject including a first respective first novel exon identified in the D) identifying from a first respective splice site coordinate in the respective fourth subset of splice site coordinates, the first respective splice site coordinate including a first corresponding donor splice site coordinate that is represented in the reference splice site coordinates for the respective principal mRNA isoform for the respective gene and a first corresponding acceptor splice site coordinate that is not represented in the reference splice site coordinates for the respective principal mRNA isoform for the respective gene.

Referring to block 760, in some embodiments, when the predicted donor site for the respective second novel exon is represented in a set of splice site coordinates for a known mRNA isoform for the respective gene, defining the respective alternative transcript as including, in order, (i) each respective exon in the respective principal mRNA isoform for the respective gene upstream of the first corresponding donor splice site, (ii) the first respective first novel exon, and (iii) each respective exon in the known mRNA isoform downstream of the predicted donor splice site.

Referring to block 762, in some embodiments, when the predicted donor site for the first respective first novel exon is not represented in the set of splice site coordinates for the known mRNA isoform for the respective gene, the method further includes identifying a second respective splice site coordinate in the respective fourth subset of splice site coordinates that includes the predicted donor site.

Referring to block 764, in some embodiments, when the corresponding acceptor splice site for the second respective splice site coordinate is not represented in the reference splice site coordinates for the respective principal mRNA isoform for the respective gene, defining the respective alternative transcript as including, in order, (i) each respective exon in the respective principal mRNA isoform for the respective gene upstream of the first corresponding donor splice site, (ii) the first respective second novel exon, and (iii) a second respective first novel exon identified in the D) identifying from the second respective splice site coordinate.

Referring to block 766, in some embodiments, when the corresponding acceptor splice site for the second respective splice site coordinate is represented in the reference splice site coordinates for the respective principal mRNA isoform for the respective gene, defining the respective alternative transcript as including, in order, (i) each respective exon in the respective principal mRNA isoform for the respective gene upstream of the first corresponding donor splice site, (ii) the respective first novel exon, and (iii) each respective exon in the respective principal mRNA isoform for the respective gene downstream of the acceptor splice site representative of the acceptor splice site for the second respective splice site coordinate.

Referring to block 768, in some embodiments, the known mRNA isoform for the respective gene is the respective principal mRNA isoform for the respective gene. Referring to block 770, in some embodiments, the known mRNA isoform for the respective gene is selected from a plurality of known mRNA isoforms for the respective gene.

Referring to block 772, in some embodiments, defining, for a respective gene in the first set of one or more genes, a respective alternative transcript for the respective gene in the biological sample from the test subject including a first respective second novel exon identified in the D) identifying from a second respective splice site coordinate in the respective fourth subset of splice site coordinates, the second respective splice site coordinate including a second corresponding donor splice site coordinate that is not represented in the reference splice site coordinates for the respective principal mRNA isoform for the respective gene and a second corresponding acceptor splice site coordinate that is represented in the reference splice site coordinates for the respective principal mRNA isoform for the respective gene.

Referring to block 774, in some embodiments, when the predicted acceptor site for the respective second novel exon is represented in a set of splice site coordinates for a known mRNA isoform for the respective gene, defining the respective alternative transcript as including, in order, (i) each respective exon of the known mRNA isoform upstream of the predicted acceptor splice site, (ii) the first respective second novel exon, and (iii) each respective exon in the respective principal mRNA isoform for the respective gene downstream of the first corresponding acceptor splice site.

Referring to block 776, in some embodiments, when the predicted acceptor site for the first respective second novel exon is not represented in the set of splice site coordinates for the known mRNA isoform for the respective gene, identifying a third respective splice site coordinate in the respective fourth subset of splice site coordinates that includes the predicted acceptor site.

Referring to block 778, in some embodiments, when the corresponding donor splice site for the second respective splice site coordinate is not represented in the reference splice site coordinates for the respective principal mRNA isoform for the respective gene, defining the respective alternative transcript as including, in order, (i) a second respective second novel exon identified in the D) identifying from the second respective splice site coordinate, (ii) the first respective second novel exon, and (iii) each respective exon in the respective principal mRNA isoform for the respective gene downstream of the first corresponding acceptor splice site.

Referring to block 780, in some embodiments, when the corresponding donor splice site for the second respective splice site coordinate is represented in the reference splice site coordinates for the respective principal mRNA isoform for the respective gene, defining the respective alternative transcript as including, in order, (i) each respective exon in the respective principal mRNA isoform for the respective gene upstream of the donor splice site representative of the donor splice site for the second respective splice site coordinate, (ii) the respective second novel exon, and (iii) each respective exon in the respective principal mRNA isoform for the respective gene downstream of the first corresponding acceptor splice site.

Referring to block 782, in some embodiments, the known mRNA isoform for the respective gene is the respective principal mRNA isoform for the respective gene. Referring to block 784, in some embodiments, the known mRNA isoform for the respective gene is selected from a plurality of known mRNA isoforms for the respective gene.

Referring to block 784, in some embodiments, the method includes generating a respective isoform library for a respective gene in the set of one or more genes, the respective isoform library including one or more known mRNA isoforms for the respective gene and one or more respective alternative transcript for the respective gene defined from a respective novel exon identified.

Referring to block 786, in some embodiments, generating a splicing graph for the respective gene based on the respective isoform library. Referring to block 788, in some embodiments, the splicing graph is further based on one or more alternative splicing events defined by the respective third subset of the respective plurality of splice site coordinates. In some embodiments, the splicing graph is a directed acyclic graph (DAG), where splice sites are nodes and edges are the connections between splice sites. Splice sites can be connected by introns or exons. Accordingly, in some embodiments, the nodes of the splice graph are connected with exons, to represent alternative splicing events, novel exons, and/or novel transcripts detected in the sequencing data. Splice sites can be identified, e.g., with the genomic coordinates, or with sequential integers from the 5′ to the 3′ end of the transcript. The order of splice sites depends on the strand of the transcript, so for transcripts on the positive strand, it will reflect ascending genomics coordinates, while for transcripts on the negative strand, the order will reflect descending genomics coordinates.

Referring to block 790, in some embodiments, the method includes generating a report including whether the biological sample included an alternative splicing event for one or more genes in the first set of one or more genes.

The results of the bioinformatics pipeline may be provided for report generation 208. Report generation may comprise variant science analysis, including the interpretation of variants (including somatic and germline variants as applicable) for pathogenic and biological significance. The variant science analysis may also estimate microsatellite instability (MSI) or tumor mutational burden. Targeted treatments may be identified based on alternate splicing patterns, gene, variant, and cancer type, for further consideration and review by the ordering physician. In some aspects, clinical trials may be identified for which the patient may be eligible, based on alternate splicing patterns, mutations, cancer type, and/or clinical history. A validation step may occur, after which the report may be finalized for sign-out and delivery. In some embodiments, a first or second report may include additional data provided through a clinical dataflow 202, such as patient progress notes, pathology reports, imaging reports, and other relevant documents. Such clinical data is ingested, reviewed, and abstracted based on a predefined set of curation rules. The clinical data is then populated into the patient's clinical history timeline for report generation.

Further details on clinical report generation are disclosed in US Patent Publication No. 2020/0255909, and published Aug. 13, 2020, which is hereby incorporated herein by reference in its entirety.

One of skill in the art will appreciate that any of a wide array of different computer topologies are used for the application and all such topologies are within the scope of the present disclosure.

The systems and methods disclosed herein are further illustrated by the following non-limiting examples.

### Digital and Laboratory Health Care Platform:

The methods and systems described above may be utilized in combination with or as part of a digital and laboratory health care platform that is generally targeted to medical care and research. It should be understood that many uses of the methods and systems described above, in combination with such a platform, are possible. One example of such a platform is described in U.S. Patent Publication No. 2021/0090694, titled “Data Based Cancer Research and Treatment Systems and Methods”, and published Mar. 25, 2021, which is incorporated herein by reference and in its entirety for any and all purposes.

For example, an implementation of one or more embodiments of the methods and systems as described above may include microservices constituting a digital and laboratory health care platform supporting splicing analysis of mRNA sequencing data. Embodiments may include a single microservice for executing and delivering splicing analysis of mRNA sequencing data or may include a plurality of microservices each having a particular role which together implement one or more of the embodiments above. In one example, a first microservice may execute mRNA sequencing in order to deliver mRNA sequencing data to a second microservice for splicing analysis of mRNA sequencing data. Similarly, the second microservice may execute mRNA sequencing to deliver splicing analysis of mRNA sequencing data according to an embodiment, above.

Where embodiments above are executed in one or more micro-services with or as part of a digital and laboratory health care platform, one or more of such micro-services may be part of an order management system that orchestrates the sequence of events as needed at the appropriate time and in the appropriate order necessary to instantiate embodiments above. A micro-services based order management system is disclosed, for example, in U.S. Patent Publication No. 2020/80365232, titled “Adaptive Order Fulfillment and Tracking Methods and Systems”, and published Nov. 19, 2020, which is incorporated herein by reference and in its entirety for all purposes.

For example, continuing with the above first and second microservices, an order management system may notify the first microservice that an order for mRNA sequencing has been received and is ready for processing. The first microservice may execute and notify the order management system once the delivery of mRNA sequencing data is ready for the second microservice. Furthermore, the order management system may identify that execution parameters (prerequisites) for the second microservice are satisfied, including that the first microservice has completed, and notify the second microservice that it may continue processing the order to splicing analysis of mRNA sequencing data according to an embodiment, above.

Where the digital and laboratory health care platform further includes a genetic analyzer system, the genetic analyzer system may include targeted panels and/or sequencing probes. An example of a targeted panel is disclosed, for example, in U.S. Patent Publication No. 2021/0090694, titled “Data Based Cancer Research and Treatment Systems and Methods”, and published Mar. 25, 2021, which is incorporated herein by reference and in its entirety for all purposes. An example of a targeted panel for sequencing cell-free (cf) DNA and determining various characteristics of a specimen based on the sequencing is disclosed, for example, in U.S. Patent Publication No. 2021/0343372, titled “Methods And Systems For Dynamic Variant Thresholding In A Liquid Biopsy Assay”, and published Nov. 4, 2021, U.S. Patent Publication No. 2021/0257055, titled “Estimation Of Circulating Tumor Fraction Using Off-Target Reads Of Targeted-Panel Sequencing”, published Aug. 19, 2021, and issued as U.S. Pat. No. 11,211,147, and U.S. Patent Publication No. 2021/0257047, titled “Methods And Systems For Refining Copy Number Variation In A Liquid Biopsy Assay”, published Aug. 19, 2021, and issued as U.S. Pat. No. 11,211,144, which are each incorporated herein by reference and in their entireties for all purposes. In one example, targeted panels may enable the delivery of next generation sequencing results (including sequencing of DNA and/or RNA from solid or cell-free specimens) for splicing analysis of mRNA sequencing data according to an embodiment, above. An example of the design of next-generation sequencing probes is disclosed, for example, in U.S. Patent Publication No. 2021/0115511, titled “Systems and Methods for Next Generation Sequencing Uniform Probe Design”, and published Jun. 22, 2021, and U.S. Patent Publication No. 2021/0269878, titled “Systems and Methods for Next Generation Sequencing Uniform Probe Design”, and published Sep. 2, 2021, which are each incorporated herein by reference and in their entireties for all purposes.

Where the digital and laboratory health care platform further includes an epigenetic analyzer system, the epigenetic analyzer system may analyze specimens to determine their epigenetic characteristics and may further use that information for monitoring a patient over time. An example of an epigenetic analyzer system is disclosed, for example, in U.S. Patent Publication No. 2021/0398617, titled “Molecular Response And Progression Detection From Circulating Cell Free DNA”, and published Dec. 23, 2021, which is incorporated herein by reference and in its entirety for all purposes.

Where the digital and laboratory health care platform further includes a bioinformatics pipeline, the methods and systems described above may be utilized after completion or substantial completion of the systems and methods utilized in the bioinformatics pipeline. As one example, the bioinformatics pipeline may receive next-generation genetic sequencing results and return a set of binary files, such as one or more BAM files, reflecting DNA and/or RNA read counts aligned to a reference genome. The methods and systems described above may be utilized, for example, to ingest the DNA and/or RNA read counts and produce splicing analysis of mRNA sequencing data as a result.

When the digital and laboratory health care platform further includes an RNA data normalizer, any RNA read counts may be normalized before processing embodiments as described above. An example of an RNA data normalizer is disclosed, for example, in U.S. Patent Publication No. 2020/0098448, titled “Methods of Normalizing and Correcting RNA Expression Data”, and published Mar. 26, 2020, which is incorporated herein by reference and in its entirety for all purposes.

When the digital and laboratory health care platform further includes a genetic data deconvolver, any system and method for deconvolving may be utilized for analyzing genetic data associated with a specimen having two or more biological components to determine the contribution of each component to the genetic data and/or determine what genetic data would be associated with any component of the specimen if it were purified. An example of a genetic data deconvolver is disclosed, for example, in U.S. Patent Publication No. 2020/0210852, published Jul. 2, 2020, and PCT/US19/69161, filed Dec. 31, 2019, both titled “Transcriptome Deconvolution of Metastatic Tissue Samples”; and U.S. Patent Publication No. 2021/0118526, titled “Calculating Cell-type RNA Profiles for Diagnosis and Treatment”, and published Apr. 22, 2021, the contents of each of which are incorporated herein by reference and in their entireties for all purposes.

RNA expression levels may be adjusted to be expressed as a value relative to a reference expression level. Furthermore, multiple RNA expression data sets may be adjusted, prepared, and/or combined for analysis and may be adjusted to avoid artifacts caused when the data sets have differences because they have not been generated by using the same methods, equipment, and/or reagents. An example of RNA data set adjustment, preparation, and/or combination is disclosed, for example, in U.S. Patent Publication No. 2022/0059190, titled “Systems and Methods for Homogenization of Disparate Datasets”, and published Feb. 24, 2022, which is incorporated herein by reference and in its entirety for all purposes.

When the digital and laboratory health care platform further includes an automated RNA expression caller, RNA expression levels associated with multiple samples may be compared to determine whether an artifact is causing anomalies in the data. An example of an automated RNA expression caller is disclosed, for example, in U.S. Pat. No. 11,043,283, titled “Systems and Methods for Automating RNA Expression Calls in a Cancer Prediction Pipeline”, and issued Jun. 22, 2021, which is incorporated herein by reference and in its entirety for all purposes.

The digital and laboratory health care platform may further include one or more insight engines to deliver information, characteristics, or determinations related to a disease state that may be based on genetic and/or clinical data associated with a patient, specimen and/or organoid. Exemplary insight engines may include a tumor of unknown origin (tumor origin) engine, a human leukocyte antigen (HLA) loss of homozygosity (LOH) engine, a tumor mutational burden engine, a PD-L1 status engine, a homologous recombination deficiency engine, a cellular pathway activation report engine, an immune infiltration engine, a microsatellite instability engine, a pathogen infection status engine, a T cell receptor or B cell receptor profiling engine, a line of therapy engine, a metastatic prediction engine, an IO progression risk prediction engine, and so forth.

An example tumor origin or tumor of unknown origin engine is disclosed, for example, in U.S. Patent Publication No. 2020/0365268, titled “Systems and Methods for Multi-Label Cancer Classification”, and published Nov. 19, 2020, which is incorporated herein by reference and in its entirety for all purposes.

An example of an HLA LOH engine is disclosed, for example, in U.S. Pat. No. 11,081,210, titled “Detection of Human Leukocyte Antigen Class I Loss of Heterozygosity in Solid Tumor Types by NGS DNA Sequencing”, and issued Aug. 3, 2021, which is incorporated herein by reference and in its entirety for all purposes. An additional example of an HLA LOH engine is disclosed, for example, in U.S. Patent Publication No. 2021/0327536, titled “Detection of Human Leukocyte Antigen Loss of Heterozygosity”, and published Oct. 21, 2021, which is incorporated herein by reference and in its entirety for all purposes.

An example of a tumor mutational burden (TMB) engine is disclosed, for example, in U.S. Patent Publication No. 2020/0258601, titled “Targeted-Panel Tumor Mutational Burden Calculation Systems and Methods”, and published Aug. 13, 2020, which is incorporated herein by reference and in its entirety for all purposes.

An example of a PD-L1 status engine is disclosed, for example, in U.S. Patent Publication No. 2020/0395097, titled “A Pan-Cancer Model to Predict The PD-L1 Status of a Cancer Cell Sample Using RNA Expression Data and Other Patient Data”, and published Dec. 17, 2020, which is incorporated herein by reference and in its entirety for all purposes. An additional example of a PD-L1 status engine is disclosed, for example, in U.S. Pat. No. 10,957,041, titled “Determining Biomarkers from Histopathology Slide Images”, issued Mar. 23, 2021, which is incorporated herein by reference and in its entirety for all purposes.

An example of a homologous recombination deficiency engine is disclosed, for example, in U.S. Pat. No. 10,975,445, titled “An Integrative Machine-Learning Framework to Predict Homologous Recombination Deficiency”, and issued Apr. 13, 2021, which is incorporated herein by reference and in its entirety for all purposes. An additional example of a homologous recombination deficiency engine is disclosed, for example, in U.S. Pat. No. 11,164,655, titled “Systems and Methods for Predicting Homologous Recombination Deficiency Status of a Specimen”, and issued Nov. 2, 2021, which is incorporated herein by reference and in its entirety for all purposes.

An example of a cellular pathway activation report engine is disclosed, for example, in U.S. Patent Publication No. 2021/0057042, titled “Systems And Methods For Detecting Cellular Pathway Dysregulation In Cancer Specimens”, and published Feb. 25, 2021, which is incorporated herein by reference and in its entirety for all purposes.

An example of an immune infiltration engine is disclosed, for example, in U.S. Patent Publication No. 2020/0075169, titled “A Multi-Modal Approach to Predicting Immune Infiltration Based on Integrated RNA Expression and Imaging Features”, and published Mar. 5, 2020, which is incorporated herein by reference and in its entirety for all purposes.

An example of an MSI engine is disclosed, for example, in U.S. Patent Publication No. 2020/0118644, titled “Microsatellite Instability Determination System and Related Methods”, and published Apr. 16, 2020, which is incorporated herein by reference and in its entirety for all purposes. An additional example of an MSI engine is disclosed, for example, in U.S. Patent Publication No. 2021/0098078, titled “Systems and Methods for Detecting Microsatellite Instability of a Cancer Using a Liquid Biopsy”, and published Apr. 1, 2021, which is incorporated herein by reference and in its entirety for all purposes.

An example of a pathogen infection status engine is disclosed, for example, in U.S. Pat. No. 11,043,304, titled “Systems And Methods For Using Sequencing Data For Pathogen Detection”, and issued Jun. 22, 2021, which is incorporated herein by reference and in its entirety for all purposes. Another example of a pathogen infection status engine is disclosed, for example, in WO 2021/168143, titled “Systems And Methods For Detecting Viral DNA From Sequencing”, and filed Feb. 18, 2021, which is incorporated herein by reference and in its entirety for all purposes.

An example of a T cell receptor or B cell receptor profiling engine is disclosed, for example, in U.S. Pat. No. 11,414,700, titled “TCR/BCR Profiling Using Enrichment with Pools of Capture Probes”, and issued Nov. 18, 2021, which is incorporated herein by reference and in its entirety for all purposes.

An example of a line of therapy engine is disclosed, for example, in U.S. Patent Publication No. 2021/0057071, titled “Unsupervised Learning And Prediction Of Lines Of Therapy From High-Dimensional Longitudinal Medications Data”, and published Feb. 25, 2021, which is incorporated herein by reference and in its entirety for all purposes.

An example of a metastatic prediction engine is disclosed, for example, in U.S. Pat. No. 11,145,416, titled “Predicting likelihood and site of metastasis from patient records”, and issued Oct. 12, 2021, which is incorporated herein by reference and in its entirety for all purposes.

An example of an IO progression risk prediction engine is disclosed, for example, in U.S. Patent Publication No. 2022/0154284, titled “Determination of Cytotoxic Gene Signature and Associated Systems and Methods For Response Prediction and Treatment”, and published May 19, 2022, which is incorporated herein by reference and in its entirety for all purposes.

When the digital and laboratory health care platform further includes a report generation engine, the methods and systems described above may be utilized to create a summary report of a patient's genetic profile and the results of one or more insight engines for presentation to a physician. For instance, the report may provide to the physician information about the extent to which the specimen that was sequenced contained tumor or normal tissue from a first organ, a second organ, a third organ, and so forth. For example, the report may provide a genetic profile for each of the tissue types, tumors, or organs in the specimen. The genetic profile may represent genetic sequences present in the tissue type, tumor, or organ and may include variants, expression levels, information about gene products, or other information that could be derived from genetic analysis of a tissue, tumor, or organ.

The report may include therapies and/or clinical trials matched based on a portion or all of the genetic profile or insight engine findings and summaries. For example, the therapies may be matched according to the systems and methods disclosed in U.S. Patent Publication No. 2022/0208305, titled “Artificial Intelligence Driven Therapy Curation and Prioritization”, and published Jun. 30, 2022, which is incorporated herein by reference and in its entirety for all purposes. For example, the clinical trials may be matched according to the systems and methods disclosed in U.S. Patent Publication No. 2020/0381087, titled “Systems and Methods of Clinical Trial Evaluation”, published Dec. 3, 2020, which is incorporated herein by reference and in its entirety for all purposes.

The report may include a comparison of the results (for example, molecular and/or clinical patient data) to a database of results from many specimens. An example of methods and systems for comparing results to a database of results are disclosed in U.S. Patent Publication No. 2020/0135303 titled “User Interface, System, And Method For Cohort Analysis” and published Apr. 30, 2020, and U.S. Patent Publication No. 2020/0211716 titled “A Method and Process for Predicting and Analyzing Patient Cohort Response, Progression and Survival”, and published Jul. 2, 2020, which is incorporated herein by reference and in its entirety for all purposes. The information may be used, sometimes in conjunction with similar information from additional specimens and/or clinical response information, to match therapies likely to be successful in treating a patient, discover biomarkers or design a clinical trial.

Any data generated by the systems and methods and/or the digital and laboratory health care platform may be downloaded by the user. In one example, the data may be downloaded as a CSV file comprising clinical and/or molecular data associated with tests, data structuring, and/or other services ordered by the user. In various embodiments, this may be accomplished by aggregating clinical data in a system backend, and making it available via a portal. This data may include not only variants and RNA expression data, but also data associated with immunotherapy markers such as MSI and TMB, as well as RNA fusions.

When the digital and laboratory health care platform further includes a device comprising a microphone and speaker for receiving audible queries or instructions from a user and delivering answers or other information, the methods and systems described above may be utilized to add data to a database the device can access. An example of such a device is disclosed, for example, in U.S. Patent Publication No. 2020/0335102, titled “Collaborative Artificial Intelligence Method And System”, and published Oct. 22, 2020, which is incorporated herein by reference and in its entirety for all purposes.

When the digital and laboratory health care platform further includes a mobile application for ingesting patient records, including genomic sequencing records and/or results even if they were not generated by the same digital and laboratory health care platform, the methods and systems described above may be utilized to receive ingested patient records. An example of such a mobile application is disclosed, for example, in U.S. Pat. No. 10,395,772, titled “Mobile Supplementation, Extraction, And Analysis Of Health Records”, and issued Aug. 27, 2019, which is incorporated herein by reference and in its entirety for all purposes. Another example of such a mobile application is disclosed, for example, in U.S. Pat. No. 10,902,952, titled “Mobile Supplementation, Extraction, And Analysis Of Health Records”, and issued Jan. 26, 2021, which is incorporated herein by reference and in its entirety for all purposes. Another example of such a mobile application is disclosed, for example, in U.S. Patent Publication No. 2021/0151192, titled “Mobile Supplementation, Extraction, And Analysis Of Health Records”, and filed May 20, 2021, which is incorporated herein by reference and in its entirety for all purposes.

When the digital and laboratory health care platform further includes organoids developed in connection with the platform (for example, from the patient specimen), the methods and systems may be used to further evaluate genetic sequencing data derived from an organoid and/or the organoid sensitivity, especially to therapies matched based on a portion or all of the information determined by the systems and methods, including predicted cancer type(s), likely tumor origin(s), etc. These therapies may be tested on the organoid, derivatives of that organoid, and/or similar organoids to determine an organoid's sensitivity to those therapies. Any of the results may be included in a report. If the organoid is associated with a patient specimen, any of the results may be included in a report associated with that patient and/or delivered to the patient or patient's physician or clinician. In various examples, organoids may be cultured and tested according to the systems and methods disclosed in U.S. Patent Publication No. 2021/0155989, titled “Tumor Organoid Culture Compositions, Systems, and Methods”, published May 27, 2021; WO2021081253, titled “Systems and Methods for Predicting Therapeutic Sensitivity”, published Apr. 29, 2021; U.S. Patent Publication No. 2021/0172931, titled “Large Scale Organoid Analysis”, published Jun. 10, 2021; WO 2021/113821, titled “Systems and Methods for High Throughput Drug Screening”, and published Jun. 10, 2021, and U.S. Patent Publication No. 2021/0325308, titled “Artificial Fluorescent Image Systems and Methods”, and published Oct. 21, 2021, which are each incorporated herein by reference and in their entirety for all purposes. In one example, the drug sensitivity assays may be especially informative if the systems and methods return results that match with a variety of therapies, or multiple results (for example, multiple equally or similarly likely cancer types or tumor origins), each matching with at least one therapy.

When the digital and laboratory health care platform further includes application of one or more of the above in combination with or as part of a medical device or a laboratory developed test that is generally targeted to medical care and research, such laboratory developed test or medical device results may be enhanced and personalized through the use of artificial intelligence. An example of laboratory developed tests, especially those that may be enhanced by artificial intelligence, is disclosed, for example, in U.S. Patent Publication No. 2021/0118559, titled “Artificial Intelligence Assisted Precision Medicine Enhancements to Standardized Laboratory Diagnostic Testing”, and published Apr. 22, 2021, which is incorporated herein by reference and in its entirety for all purposes.

It should be understood that the examples given above are illustrative and do not limit the uses of the systems and methods described herein in combination with a digital and laboratory health care platform.

### EXAMPLES

### Example 1

EGFR (Epidermal Growth Factor Receptor)

43-year old male had a biopsy of a left-sided brain tumor which was diagnosed as glioblastoma. Ordered the validated Tempus xT test on the biopsy for DNA sequencing (Beaubier et al., Oncotarget 10, 2384-2396 (2019). RNA from the biopsy was sequenced with IDT's xGen Exome Research Panel v1.0 (IDT, Coralville, Iowa) and subsequently resequenced using IDT's xGen Exome Research Panel v2 (IDT, Coralville, Iowa). Tumor resection was performed a month after biopsy and patient underwent radiation therapy for 3 months after surgery, followed by optune treatment. No subsequent follow up. Pathology review estimated 60% tumor purity of the biopsy. See Table 2 for the report.

TABLE 2

| Field | Results |
|---|---|
| needs_review | TRUE |
| reportable_gene | TRUE |
| gene_name | EGFR |
| domain | NA |
| exon_exclusion_read_support | 686 |
| exon_inclusion_read_support | 3059 |
| psi | 0.6904 |
| description | exon_2-7_skip |
| start | 55087058 |
| end | 55223523 |
| event_type | mes |
| chr | chr7 |
| event_start | 55209979 |
| event_end | 55221845 |
| domain_start | NA |
| domain_end | NA |
| gene_start | 55086794 |
| gene_end | 55279321 |
| transcript_id | ENST00000275493_exon_2-7_skip |
| gene_id | ENSG00000146648 |
| strand | + |
| sashimi | TRUE |
| wt_transcript_id | ENST00000397752 |
| event_id | 24c73af4246d122f1a5b9e4c0bd9c6d0 |
| source_analysis_id | [redacted] |

NA = not available

### Example 2

MET (Mesenchymal Epithelial Transition Factor) 75-year old male patient underwent thoracotomy to remove right lung adenocarcinoma two months after first X-ray and follow up imaging tests. A portion of the removed tumor was sequenced by Tempus xT (Beaubier et al., Oncotarget 10, 2384-2396 (2019). RNA from the biopsy was sequenced with IDT's xGen Exome Research Panel v1.0 (IDT, Coralville, Iowa) and subsequently resequenced using IDT's xGen Exome Research Panel v2 (IDT, Coralville, Iowa). MET exon 14 also detected via DNA mutation. First-line crizotinib. He was started on capmatinib when the FDA approved that drug, but he did not tolerate it well due to fatigue and edema, nausea, and asthenia. He was switched back to crizotinib. Pathology review estimated 50% tumor purity. See Table 3 for results.

TABLE 3

| Field | Results |
|---|---|
| needs_review | TRUE |
| reportable_gene | TRUE |
| gene_name | MET |
| domain | NA |
| exon_exclusion_read_support | 220 |
| exon_inclusion_read_support | 49 |
| psi | 0.1002 |
| description | exon_14_skip |
| start | 116411708 |
| end | 116414935 |
| event_type | aes |
| chr | chr7 |
| event_start | 116411903 |
| event_end | 116412043 |
| domain_start | NA |
| domain_end | NA |
| gene_start | 116312446 |
| gene_end | 116438440 |
| transcript_id | ENST00000397752_exon_14_skip |
| gene_id | ENSG00000105976 |
| strand | + |
| sashimi | TRUE |
| wt_transcript_id | ENST00000397752 |
| event_id | 24c73af4246d122f1a5b9e4c0bd9c6d0 |
| source_analysis_id | [redacted] |

NA = not available

### Example 3

AR (Androgen Receptor)

61-year-old male with prostate cancer 4.5 years after prostatectomy and several therapies. Sample sent to Tempus sequencing after enrollment in Lu177-PSMA-617 clinical trial. Sample is castrate resistant at the time of sequencing. DNA from the sample was sequenced by Tempus xT (Beaubier et al., Oncotarget 10, 2384-2396 (2019). RNA from the sample was sequenced with IDT's xGen Exome Research Panel v1.0 (IDT, Coralville, Iowa) and subsequently resequenced using IDT's xGen Exome Research Panel v2 (IDT, Coralville, Iowa). See Table 4 for results.

TABLE 4

| Field | Results |
|---|---|
| needs_review | TRUE |
| reportable_gene | TRUE |
| gene_name | AR |
| domain | NA |
| exon_exclusion_read_support | 12 |
| exon_inclusion_read_support | 1221 |
| psi | 0.9903 |
| description | ARv7 |
| start | 66905968 |
| end | 66950461 |
| event_type | ale |
| chr | chrX |
| event_start | 66914515 |
| event_end | 66931244 |
| domain_start | NA |
| domain_end | NA |
| gene_start | 66763878 |
| gene_end | 66950461 |
| transcript_id | ENST00000374690_ARv7 |
| gene_id | ENSG00000169083 |
| strand | + |
| sashimi | TRUE |
| wt_transcript_id | ENST00000374690 |
| event_id | 72a2c833848704a4bbcc74844701d28d |
| source_analysis_id | [redacted] |

NA = not available

### Example 4

This example provides a report for an analysis done in accordance with an embodiment of the systems and methods disclosed herein without a target sequence. See Example 2 above for patient and sample details. Table 5, column 4 provides the results.

TABLE 5

| Field | Description | Proposed Type | Example |
|---|---|---|---|
| alternative_splicing_events | Total number of alternative splicing events | int(11) | 19648 |
| | in the samples | | |
| alternative_splicing_genes | Number of genes with one or more | int(11) | 8332 |
| | alternative splicing events | | |
| mean_events_per_gene | Average number of alternative splicing | float(5, 2) | 2.4 |
| | events per gene | | |
| max_events_per_gene | Maximum number of alternative splicing | int(11) | 33 |
| | events per gene | | |
| aes_events | Number alternative single exon skipping | int(11) | 13186 |
| | events | | |
| mes_events | Number of alternative multiple exon | int(11) | 3788 |
| | skipping events | | |
| aa_events | Number of alternative acceptor events | int(11) | 929 |
| ad_events | Number of alternative donor events | int(11) | 549 |
| afe_events | Number of alternative first exon events | int(11) | 809 |
| ale_events | Number of alternative last exon events | int(11) | 286 |
| ir_events | Number of intron retention events | int(11) | 57 |
| mee_events | Number of mutually exclusive exon events | int(11) | 44 |
| source_analysis_id | Bioinformatics analysis id | varchar(1000) | [redacted] |

From the foregoing, it will be appreciated that, although specific embodiments of the systems and methods disclosed herein have been described herein for purposes of illustration, various modifications may be made without deviating from the spirit and scope of the systems and methods disclosed herein. Accordingly, the systems and methods disclosed herein are not limited except as by the appended claims.

1. A method for mapping splicing events in a test subject, the method comprising:
at a computer system comprising at least one processor and a memory storing at least one program for execution by the at least one processor:
A) obtaining, for each respective gene in a first set of one or more genes, a respective set of splice site coordinates for respective aligned sequence reads, in a plurality of aligned sequence reads for mRNA in a biological sample from a test subject, mapping to the respective gene, wherein each respective splice site coordinate in the respective splice site coordinates corresponds to a respective donor splice site and a respective acceptor splice site in the respective gene, to obtain a respective plurality of splice site coordinates for the respective gene in the plurality of sequence reads;
B) comparing, for each respective gene in the first set of one or more genes, the respective plurality of splice site coordinates to reference splice site coordinates in a respective principal mRNA isoform for the respective gene, to identify (i) a respective first subset of the respective plurality of splice site coordinates that correspond to a splice site coordinate in the principal mRNA isoform, representative of constitutional splicing events in common with the respective principal mRNA isoform, and (ii) a respective second subset of the respective plurality of splice site coordinates that do not correspond to a splice site coordinate in the principal mRNA isoform, representative of alternative splicing events not in common with the respective principal mRNA isoform;
C) determining, for each respective gene in the set of one or more genes, for each respective splice site coordinate in the respective second subset of splice site coordinates, whether the respective splice site coordinate satisfies a first criteria, wherein the first criteria is satisfied when both the respective donor site and the respective acceptor site corresponding to the respective splice site coordinate are represented in the reference splice site coordinates for the respective principal mRNA isoform for the respective gene, to identify (i) a respective third subset of the respective plurality of splice site coordinates that satisfy the first criteria, representative of alternative splicing events between donor splice sites and acceptor splice sites in common with the respective principal mRNA isoform, and (ii) a respective fourth subset of the respective plurality of splice site coordinates that do not satisfy the first criteria, representative of alternative splicing events occurring between a donor site or an acceptor site not in common with the respective principal mRNA isoform,
thereby mapping splicing events in the subject for the set of one or more respective genes.

2. The method of claim 1, further comprising:
D) identifying, for each respective gene in the set of one or more genes, for a respective splice site coordinate in the respective fourth subset of splice site coordinates, a respective non-canonical exon encoded by a respective sequence read in the plurality of sequences reads mapping to the respective gene by:
when the acceptor splice site corresponding to the respective splice site coordinate is not represented in the reference splice site coordinates for the respective principal mRNA isoform for the respective gene, identifying the acceptor splice site corresponding to the respective splice site in a genomic construct for the respective gene and searching a region of the genomic construct upstream of the acceptor splice site to identify a predicted donor splice site for the respective non-canonical exon, wherein the nucleotide sequence in the genomic construct spanning from the predicted donor splice site to the acceptor splice site defines a first non-canonical exon, and
when the donor splice site corresponding to the respective splice site coordinate is not represented in the reference splice site coordinates for the respective principal mRNA isoform for the respective gene, identifying the donor splice site corresponding to the respective splice site in a genomic construct for the respective gene and searching a region of the genomic construct downstream of the donor splice site to identify a predicted acceptor splice site for the respective non-canonical exon, wherein the nucleotide sequence in the genomic construct spanning from the donor splice site to the predicted acceptor splice site defines a second non-canonical exon,
thereby mapping exon skipping events in the subject for the set of one or more respective genes.

3-8. (canceled)

9. The method of claim 1, wherein the respective set of splice site coordinates aggregates splice site coordinates across the respective aligned sequence reads in the plurality of aligned sequence reads mapping to the respective gene.

10. The method of claim 9, wherein the respective set of splice site coordinates further comprises, for each respective splice site coordinate in the respective set of splice site coordinates, a respective count of the number of unique occurrences of the respective splice site coordinate in the plurality of sequence reads.

11-13. (canceled)

14. The method of claim 1, wherein, for a respective gene in the first set of one or more genes, the principal mRNA isoform is identified as the predominant mRNA isoform in the respective plurality of sequence reads aligned to the respective gene.

15. The method of claim 1, wherein the D) identifying further comprises:
when (i) the donor splice site corresponding to the respective splice site coordinate is not represented in the reference splice site coordinates for the respective principal mRNA isoform for the respective gene, and (ii) the searching the region of the genomic construct downstream of the donor splice site does not identify a corresponding acceptor splice site, identifying an alternative terminal exon comprising:
when the acceptor splice site corresponding to the respective splice site coordinate is represented in the reference splice site coordinates for the respective principal mRNA isoform for the respective gene, a corresponding exon in the respective principal mRNA isoform that terminates at the acceptor splice site, and
when the acceptor splice site corresponding to the respective splice site coordinate is not represented in the reference splice site coordinates for the respective principal mRNA isoform for the respective gene, the first non-canonical exon.

16. The method of claim 2, wherein the region of the genomic construct upstream of the acceptor splice site that is searched is limited to a first threshold number of nucleotides upstream of the acceptor splice site in the genomic construct.

17. The method of claim 2, wherein the region of the genomic construct downstream of the donor splice site that is searched is limited to a second threshold number of nucleotides downstream of the acceptor splice site in the genomic construct.

18-19. (canceled)

20. The method of claim 1, wherein when more than one putative corresponding donor splice site are identified in the region of the genomic construct upstream of the acceptor splice site, the respective putative corresponding donor splice site, in the more than one putative corresponding donor splice sites, closest to the acceptor splice site is identified as the corresponding donor splice site.

21. The method of claim 1, wherein when more than one putative corresponding acceptor splice site are present in the region of the genomic construct downstream of the donor splice site, the respective putative corresponding acceptor splice site, in the more than one putative corresponding acceptor splice sites, closest to the donor splice site is identified as the corresponding acceptor splice site.

22. The method of claim 1, further comprising, when a respective plurality of non-canonical exons comprising more than a first threshold number of different exons sharing a common donor splice site or a common acceptor splice site are identified for a respective gene, in the first set of one or more genes, filtering out respective non-canonical exons that are represented in the respective plurality of non-canonical exons less than a second threshold number of times.

23-24. (canceled)

25. The method of claim 22, wherein the second threshold number of times is a measure of central tendency of the number of times each respective splice site coordinate in the respective sub-plurality of respective splice site coordinates is represented in the fourth subset of splice site coordinates.

26. The method of claim 2, further comprising defining, for a respective gene in the first set of one or more genes, a respective alternative transcript for the respective gene in the biological sample from the test subject comprising a first respective first non-canonical exon identified in the D) identifying from a first respective splice site coordinate in the respective fourth subset of splice site coordinates, the first respective splice site coordinate comprising a first corresponding donor splice site coordinate that is represented in the reference splice site coordinates for the respective principal mRNA isoform for the respective gene and a first corresponding acceptor splice site coordinate that is not represented in the reference splice site coordinates for the respective principal mRNA isoform for the respective gene, by:
when the predicted donor site for the respective first non-canonical exon is represented in a set of splice site coordinates for a known mRNA isoform for the respective gene, defining the respective alternative transcript as comprising, in order, (i) each respective exon in the respective principal mRNA isoform for the respective gene upstream of the first corresponding donor splice site, (ii) the first respective first non-canonical exon, and (iii) each respective exon in the known mRNA isoform downstream of the predicted donor splice site.

27. The method of claim 26, further comprising:
when the predicted donor site for the first respective first non-canonical exon is not represented in the set of splice site coordinates for the known mRNA isoform for the respective gene, the method further comprises identifying a second respective splice site coordinate in the respective fourth subset of splice site coordinates that comprises the predicted donor site, and
when the corresponding acceptor splice site for the second respective splice site coordinate is not represented in the reference splice site coordinates for the respective principal mRNA isoform for the respective gene, defining the respective alternative transcript as comprising, in order, (i) each respective exon in the respective principal mRNA isoform for the respective gene upstream of the first corresponding donor splice site, (ii) the first respective second non-canonical exon, and (iii) a second respective first non-canonical exon identified in the D) identifying from the second respective splice site coordinate.

28. The method of claim 27, further comprising, when the corresponding acceptor splice site for the second respective splice site coordinate is represented in the reference splice site coordinates for the respective principal mRNA isoform for the respective gene, defining the respective alternative transcript as comprising, in order, (i) each respective exon in the respective principal mRNA isoform for the respective gene upstream of the first corresponding donor splice site, (ii) the respective first non-canonical exon, and (iii) each respective exon in the respective principal mRNA isoform for the respective gene downstream of the acceptor splice site representative of the acceptor splice site for the second respective splice site coordinate.

29. The method of claim 1, further comprising defining, for a respective gene in the first set of one or more genes, a respective alternative transcript for the respective gene in the biological sample from the test subject comprising a first respective second non-canonical exon identified in the D) identifying from a second respective splice site coordinate in the respective fourth subset of splice site coordinates, the second respective splice site coordinate comprising a second corresponding donor splice site coordinate that is not represented in the reference splice site coordinates for the respective principal mRNA isoform for the respective gene and a second corresponding acceptor splice site coordinate that is represented in the reference splice site coordinates for the respective principal mRNA isoform for the respective gene, by:
when the predicted acceptor site for the respective second non-canonical exon is represented in a set of splice site coordinates for a known mRNA isoform for the respective gene, defining the respective alternative transcript as comprising, in order, (i) each respective exon of the known mRNA isoform upstream of the predicted acceptor splice site, (ii) the first respective second non-canonical exon, and (iii) each respective exon in the respective principal mRNA isoform for the respective gene downstream of the first corresponding acceptor splice site.

30. The method of claim 29, further comprising:
when the predicted acceptor site for the first respective second non-canonical exon is not represented in the set of splice site coordinates for the known mRNA isoform for the respective gene, identifying a third respective splice site coordinate in the respective fourth subset of splice site coordinates that comprises the predicted acceptor site, and
when the corresponding donor splice site for the second respective splice site coordinate is not represented in the reference splice site coordinates for the respective principal mRNA isoform for the respective gene, defining the respective alternative transcript as comprising, in order, (i) a second respective second non-canonical exon identified in the D) identifying from the second respective splice site coordinate, (ii) the first respective second non-canonical exon, and (iii) each respective exon in the respective principal mRNA isoform for the respective gene downstream of the first corresponding acceptor splice site.

31. The method of claim 29, further comprising, when the corresponding donor splice site for the second respective splice site coordinate is represented in the reference splice site coordinates for the respective principal mRNA isoform for the respective gene, defining the respective alternative transcript as comprising, in order, (i) each respective exon in the respective principal mRNA isoform for the respective gene upstream of the donor splice site representative of the donor splice site for the second respective splice site coordinate, (ii) the respective second non-canonical exon, and (iii) each respective exon in the respective principal mRNA isoform for the respective gene downstream of the first corresponding acceptor splice site.

32. The method of claim 26, wherein the known mRNA isoform for the respective gene is the respective principal mRNA isoform for the respective gene.

33. (canceled)

34. The method of claim 26, further comprising generating a respective isoform library for a respective gene in the set of one or more genes, the respective isoform library comprising one or more known mRNA isoforms for the respective gene and one or more respective alternative transcript for the respective gene defined from a respective non-canonical exon identified in the D) identifying.

35-36. (canceled)

37. The method of claim 1, further comprising generating a report comprising whether the biological sample included an alternative splicing event for one or more genes in the first set of one or more genes.

38-79. (canceled)

80. A computer system comprising:
one or more processors; and
a non-transitory computer-readable medium including computer-executable instructions that, when executed by the one or more processors, cause the processors to perform a method for mapping splicing events in a test subject, the method comprising:
A) obtaining, for each respective gene in a first set of one or more genes, a respective set of splice site coordinates for respective aligned sequence reads, in a plurality of aligned sequence reads for mRNA in a biological sample from a test subject, mapping to the respective gene, wherein each respective splice site coordinate in the respective splice site coordinates corresponds to a respective donor splice site and a respective acceptor splice site in the respective gene, to obtain a respective plurality of splice site coordinates for the respective gene in the plurality of sequence reads;
B) comparing, for each respective gene in the first set of one or more genes, the respective plurality of splice site coordinates to reference splice site coordinates in a respective principal mRNA isoform for the respective gene, to identify (i) a respective first subset of the respective plurality of splice site coordinates that correspond to a splice site coordinate in the principal mRNA isoform, representative of constitutional splicing events in common with the respective principal mRNA isoform, and (ii) a respective second subset of the respective plurality of splice site coordinates that do not correspond to a splice site coordinate in the principal mRNA isoform, representative of alternative splicing events not in common with the respective principal mRNA isoform;
C) determining, for each respective gene in the set of one or more genes, for each respective splice site coordinate in the respective second subset of splice site coordinates, whether the respective splice site coordinate satisfies a first criteria, wherein the first criteria is satisfied when both the respective donor site and the respective acceptor site corresponding to the respective splice site coordinate are represented in the reference splice site coordinates for the respective principal mRNA isoform for the respective gene, to identify (i) a respective third subset of the respective plurality of splice site coordinates that satisfy the first criteria, representative of alternative splicing events between donor splice sites and acceptor splice sites in common with the respective principal mRNA isoform, and (ii) a respective fourth subset of the respective plurality of splice site coordinates that do not satisfy the first criteria, representative of alternative splicing events occurring between a donor site or an acceptor site not in common with the respective principal mRNA isoform,
thereby mapping splicing events in the subject for the set of one or more respective genes.

81. A non-transitory computer-readable storage medium having stored thereon program code instructions that, when executed by a processor, cause the processor to perform a method for mapping splicing events in a test subject, the method comprising:
A) obtaining, for each respective gene in a first set of one or more genes, a respective set of splice site coordinates for respective aligned sequence reads, in a plurality of aligned sequence reads for mRNA in a biological sample from a test subject, mapping to the respective gene, wherein each respective splice site coordinate in the respective splice site coordinates corresponds to a respective donor splice site and a respective acceptor splice site in the respective gene, to obtain a respective plurality of splice site coordinates for the respective gene in the plurality of sequence reads;
B) comparing, for each respective gene in the first set of one or more genes, the respective plurality of splice site coordinates to reference splice site coordinates in a respective principal mRNA isoform for the respective gene, to identify (i) a respective first subset of the respective plurality of splice site coordinates that correspond to a splice site coordinate in the principal mRNA isoform, representative of constitutional splicing events in common with the respective principal mRNA isoform, and (ii) a respective second subset of the respective plurality of splice site coordinates that do not correspond to a splice site coordinate in the principal mRNA isoform, representative of alternative splicing events not in common with the respective principal mRNA isoform;
C) determining, for each respective gene in the set of one or more genes, for each respective splice site coordinate in the respective second subset of splice site coordinates, whether the respective splice site coordinate satisfies a first criteria, wherein the first criteria is satisfied when both the respective donor site and the respective acceptor site corresponding to the respective splice site coordinate are represented in the reference splice site coordinates for the respective principal mRNA isoform for the respective gene, to identify (i) a respective third subset of the respective plurality of splice site coordinates that satisfy the first criteria, representative of alternative splicing events between donor splice sites and acceptor splice sites in common with the respective principal mRNA isoform, and (ii) a respective fourth subset of the respective plurality of splice site coordinates that do not satisfy the first criteria, representative of alternative splicing events occurring between a donor site or an acceptor site not in common with the respective principal mRNA isoform,
thereby mapping splicing events in the subject for the set of one or more respective genes.
